(19) United States
(12) Patent Application Publication
(10) Pub. No.: US 2022/0267769 A1
(43) Pub. Date: Aug. 25, 2022

(54) MEDICAL USES, METHODS AND USES

(71) Applicants: Myriam AOUADI, Huddinge (SE); Valerio AZZIMATO, Huddinge (SE)

(72) Inventors: Myriam AOUADI, Huddinge (SE); Valerio AZZIMATO, Huddinge (SE)

(21) Appl. No.: 17/627,587

(22) PCT Filed: Jul. 17, 2020
(86) PCT No.: PCT/EP2020/070330

§ 371 Date: Jan. 14, 2022

(30) Foreign Application Priority Data
Jul. 18, 2019 (GB) 1910299.5

(51) Int. Cl. C12N 15/113; A61P 1/16

(52) U.S. Cl. CPC C12N 15/113; A61P 1/16; C12N 2310/113; C12N 2310/14

(57) ABSTRACT

Described are agents which inhibit microRNA-144 (miR-144) for use in treating or preventing a liver disease and/or liver condition in which oxidative stress is a contributory factor in a subject, methods for identifying a subject who has, or who is at risk of developing, said liver disease and/or liver condition, methods of predicting the response of a subject with said liver disease and/or liver condition to an agent which inhibits miR-144, methods of diagnosing said liver disease and/or liver condition, pharmaceutical compositions and kits.

The invention relates to agents which inhibit microRNA-144 (miR-144) for use in treating or preventing a liver disease and/or liver condition in which oxidative stress is a contributory factor in a subject; methods for identifying a subject who has, or who is at risk of developing, said liver disease and/or liver condition; methods of predicting the response of a subject with said liver disease and/or liver condition to an agent which inhibits miR-144; methods of diagnosing said liver disease and/or liver condition; and related pharmaceutical compositions and kits.

Obesity represents a major health issue worldwide as excessive weight significantly increases the risk for several metabolic complications including non-alcoholic fatty liver disease (NAFLD), non-alcoholic steatohepatitis (NASH) and insulin resistance and type 2 diabetes (T2D). The estimated prevalence of NAFLD worldwide is approximately 25%, but the real prevalence of NAFLD and the associated disorders is unknown mainly because reliable and applicable diagnostic tests are lacking.

Lipid accumulation during obesity is associated with oxidative stress and inflammatory activation of liver macrophages. Additionally, oxidative stress in the liver has been implicated in the progression of fatty liver to NASH, fibrosis and hepatocellular carcinoma.

The main mechanism protecting against oxidative stress is the Nuclear Factor Erythroid 2-Related Factor 2 (NRF2)/ARE pathway, which induces the expression of antioxidant response genes. Inappropriate lipid accumulation leads to oxidative stress and excessive production of reactive oxygen species (ROS).

Oxidative stress is thought to be an important driver of NASH in insulin resistance and obesity. NASH is a worldwide burden and is predicted to be the leading cause for liver transplant in the next 20 years (S. Furukawa et al., Increased oxidative stress in obesity and its impact on metabolic syndrome. J Clin Invest 114, 1752-1761 (2004)). NASH can progress to cirrhosis in up to 15% of patients, and there is currently no therapy that is of proven benefit for NASH.

Previous methods to decrease oxidative stress using agents with an antioxidant activity (such as glutathione, ubichinol, and uric acid or derivatives of the diet such as vitamins C and E, carotenoids, lipoic acid, selenium) have generally failed. The lack of efficacy of these exogenous antioxidants is thought to be due to both non-specific systemic effects and a decrease in the endogenous antioxidant response.

Vitamin E is a lipophilic molecule with antioxidant activity that prevents membrane damage by ROS. The effects of vitamin E have been investigated in several experimental murine models of NAFLD showing an improvement of NASH and a reduction in oxidative stress markers, hepatic stellate cell activation, and histologic fibrosis in mice supplemented with vitamin E (Nan Y M, et al., Antioxidants vitamin E and 1-aminobenzotriazole prevent experimental non-alcoholic steatohepatitis in mice. Scand J Gastroenterol. 2009; 44:1121-1131; Phung N. et al., Pro-oxidant-mediated hepatic fibrosis and effects of antioxidant intervention in murine dietary steatohepatitis. Int J Mol Med. 2009; 24:171-180; and Pacana T. et al., Vitamin E and nonalcoholic fatty liver disease. Curr Opin Clin Nutr Metab Care. 2012; 15:641-648).

Additionally, the effects of vitamin E, or vitamin E in combination with other drugs, on liver damage in patients with biopsy-proven NASH, have been investigated in a few small studies, however conflicting results were observed (Sanyal A J et al., Pioglitazone, vitamin E, or placebo for nonalcoholic steatohepatitis. N Engl J Med. 2010; 362:1675-1685; Harrison S A et al., Vitamin E and vitamin C treatment improves fibrosis in patients with nonalcoholic steatohepatitis. Am J Gastroenterol. 2003; 98:2485-2490; and Dufour J F et al., Randomized placebo-controlled trial of ursodeoxycholic acid with vitamin E in nonalcoholic steatohepatitis. Clin Gastroenterol Hepatol. 2006; 4:1537-1543).

Recently, two large Multicenter Randomized Controlled Trials investigated the efficacy of vitamin E in subjects with NAFLD. In the “PIVENS” trial, in adult patients with aggressive NASH and without diabetes or cirrhosis, high-dose vitamin E supplementation (800 UI q.d.) significantly improved NASH histology compared to pioglitazone or placebo treatment groups; however, increased insulin resistance and plasma triglyceride levels were reported (Sanyal A J et al., 2010). In contrast, the “TONIC” trial found that neither vitamin E nor metformin was superior to placebo in attaining the primary outcome of sustained reduction in ALT level in patients with paediatric NAFLD (Lavine J E et al., Effect of vitamin E or metformin for treatment of nonalcoholic fatty liver disease in children and adolescents: the TONIC randomized controlled trial. JAMA. 2011; 305:1659-1668).

Therefore, even though there are some reports about the efficacy of vitamin E supplementation in NAFLD/NASH, there are concerns about its safety. Additionally, caution is needed when interpreting the effects of antioxidants as due solely or even primarily to the antioxidant nature of the compounds. A related problem is the non-standard dosing of even well-established antioxidant compounds. Agents such as vitamin E, vitamin C and coenzyme Q function because they are single electron acceptors, but can also act as single electron donors which are highly reactive. At high concentrations both vitamin C and coenzyme Q can be pro-oxidants and have potential to cause liver damage (Abudu N et al., Vitamins in human arteriosclerosis with emphasis on vitamin C and vitamin E. Clin Chim Acta. 2004; 339:11-25).

The lack of beneficial effects, and in some cases, the observation of deleterious effects highlights the importance for new treatment methods. Additionally, the current diagnosis of liver disease involves invasive techniques, and so non-invasive techniques which could diagnose oxidative stress and identify a patient predisposed to insulin resistance, Type 2 diabetes, NASH and hepatocellular carcinoma would be beneficial.

Against this background, the inventors have surprisingly found that silencing of a specific microRNA (miRNA) in the liver, namely miR-144, decreases oxidative stress in the liver by increasing the antioxidant response. The inventors' findings have therefore identified a novel therapy for liver diseases and/or liver conditions. Targeting the endogenous antioxidant response (rather than using exogenous antioxidant that ultimately block the endogenous response) provides an attractive therapy for liver insulin resistance and NASH.

Additionally, the inventors have surprisingly found that this miRNA could be easily measured for diagnosis of liver oxidative stress which predispose for liver diseases and/or liver conditions such as NASH.

Accordingly, in a first aspect the invention provides an agent that inhibits microRNA-144 (miR-144) for use in treating or preventing a liver disease and/or liver condition in which oxidative stress is a contributory factor in a subject.

In a second aspect, the invention provides use of an agent that inhibits microRNA-144 (miR-144) for the manufacture of a medicament for treating or preventing a liver disease and/or liver condition in which oxidative stress is a contributory factor in a subject.

In a third aspect, the invention provides a method for treating or preventing a liver disease and/or liver condition in which oxidative stress is a contributory factor in a subject, wherein the method comprises administering an agent that inhibits microRNA-144 (miR-144) to the subject.

As described herein, the inventors have surprisingly found that Nuclear factor erythroid 2-related factor 2 (NRF2) in the liver is regulated by a miRNA (miR-144) expressed at high levels by liver macrophages and in blood of obese insulin resistant patients. Specific silencing of miR-144 in liver macrophages in obese mice increased NRF2 protein levels, resulting in decreased ROS release by both macrophages and hepatocytes and an overall decrease in oxidative stress and glucose intolerance.

As described in the accompanying Example, the anti-oxidant defence is ineffective in the liver of obese insulin resistant patients but not in lean, or obese insulin sensitive individuals. This was due to a dramatic decrease of Nuclear factor erythroid 2-related factor 2 (NRF2) protein levels in the livers of obese insulin resistant humans and mice compared with healthy controls.

Nuclear factor erythroid 2-related factor 2 (NRF2; also termed “NFE2L2” and “Nrf2”), is a basic leucine zipper transcription factor and a master regulator of redox homeostasis. Under normal physiological conditions, NRF2 is targeted to proteasomal degradation through its association with Kelch-like ECH-associated protein-1 (KEAP1). Conversely, upon oxidative stress this complex dissociates and NRF2 translocates to the nucleus where it binds to the antioxidant responsive element (ARE), thus driving the antioxidant response.

miRNAs are small (typically 17 to 27 nucleotides), non-coding RNAs involved in post-transcriptional regulation of gene expression in multicellular organisms by affecting both the stability and translation of mRNAs. The miRNAs are each processed from a longer precursor RNA molecule (“precursor miRNA”). Precursor miRNAs are transcribed from non-protein-encoding genes. A precursor may have a length of at least 50, 60, 66, 70, 75, 80, 85, 100, 150, 200 nucleotides or more. The precursor miRNAs have two regions of complementarity that enables a stem-loop- or fold-back-like structure to form, which in animals is cleaved by enzymes called Dicer and Drosha. Dicer and Drosha are ribonuclease III-like nucleases. The processed miRNA is typically a portion of the stem.

The processed miRNA (also referred to as “mature miRNA”) is incorporated into a large complex known as the RNA-induced silencing complex (RISC), and in animals, miRNA-based gene modulation occurs predominantly by the mature miRNA binding to an mRNA target site through partial base pairing, resulting in translational inhibition or destabilization of the target mRNA.

By the term “MicroRNA”, or “miRNA” we include a single-stranded, or double stranded non-coding RNA, at least about 6 nucleotides in length that can regulate gene expression at the post-transcriptional level by either degrading a target mRNA or inhibiting their translation.

miR-144 is a miRNA expressed at high levels by liver macrophages and in blood of obese insulin resistant patients. The biological activity or biological action of miR-144, refers to any function(s) exhibited or performed by a naturally occurring, and/or wild type form of miR-144 as measured or observed in vivo (i.e. in the natural physiological environment of the protein) or in vitro (i.e. under laboratory conditions).

Biological activities of miR-144 include, but are not limited to, decreasing the protein levels of its target NRF2 and therefore the expression of NRF2 target genes (such as those in Table 3 (S9).

The biological activity of miR-144 can be measured by methods known in the art, including but not limited to measurement of NRF2 protein levels by western blot and/or ELISA using an antibody against NRF2, measurement of NRF2 target genes by real-time PCR, and/or measurement of oxidative stress by detection of reactive oxygen species as disclosed herein.

By “an agent that inhibits microRNA-144 (miR-144)” we include the meaning of any compound which inhibits (e.g., downregulates, antagonizes, suppresses, reduces, prevents, decreases, blocks, and/or reverses) the expression and/or biological activity and/or effect of miR-144. More particularly, an inhibitor can act in a manner such that the biological activity of miR-144 is decreased in a manner that is antagonistic (e.g., against, a reversal of, contrary to) to the natural, wild type, action of miR-144.

In a preferred embodiment, the agent is cell-permeable, cannot be rapidly excreted, is stable in vivo, and binds to miR-144 with high specificity and affinity.

In an embodiment, the agent may be one that selectively inhibits miR-144. For example, the agent may inhibit and/or decrease the expression and/or biological activity of miR-144 to a greater extent than it inhibits an unrelated miRNA, such as miR-532. As shown in the accompanying Examples, silencing miR-144 with the antagomiR agent had no effect on miR-532. Preferably, the agent inhibits and/or decreases the expression and/or biological activity of miR-144 at least 5, or at least 10, or at least 50 times more than it inhibits another unrelated miRNA. More preferably, the agent inhibits and/or decreases the expression and/or biological activity of miR-144 at least 100, or at least 1,000, or at least 10,000 times more than it inhibits another unrelated miRNA.

By “treating” or “treatment” we include administering therapy to reverse, reduce, alleviate, arrest or cure the symptoms, clinical signs, and/or underlying pathology of a specific disorder, disease, injury or condition in a manner to improve or stabilise a subject's disease. Thus, treatment refers to administration of the agent to a patient in need thereof, with the expectation that they will obtain a therapeutic benefit.

“Treating” or “treatment” of a liver disease and/or liver condition in which oxidative stress is a contributory factor in a subject includes improvement of one or more of: hepatocyte death, immune cell infiltration and/or fibrosis. In the context of the present invention, treatment may include upregulating the antioxidant response in the liver of the subject. As such, a therapeutic benefit can be achieved without curing a particular disease or condition, but rather, preferably encompasses a result which includes one or more of alleviation of the disease or condition, reduction of a symptom associated with the disease or condition, elimination of the disease or condition, prevention or alleviation of a secondary disease or condition resulting from the occurrence of a primary disease or condition (e.g. hepatocellular carcinoma resulting from the progression of NASH), and/or prevention of the disease or condition. A therapeutic benefit can be assessed by one of ordinary skill in the art and/or by a trained clinician who is treating the subject.

The term “preventing” is art-recognised, and when used in relation to a condition, such as a liver disease and/or liver condition or any other medical condition, it includes administration of an agent/composition which reduces the frequency of, or delays the onset of, symptoms, clinical signs, and/or underlying pathology of a specific disorder, disease, injury or medical condition in an subject relative to an individual who does not receive the molecule/composition. The term “prophylactic” treatment is art-recognised and is used interchangeably with “preventing” and “prevention”. “Prophylactic treatment” includes administration of a molecule/compound prior to clinical manifestation of the unwanted condition (e.g. NASH) (i.e. it protects the individual against developing the unwanted condition), whereas if it is administered after manifestation of the unwanted condition, the treatment is therapeutic (i.e. it is intended to diminish, ameliorate, or stabilise the existing unwanted condition or associated side effects).

In the context of the present invention, “preventing” a liver disease and/or liver condition, may also include preventing the progression of one form of a liver disease and/or liver condition to a more severe liver disease and/or condition.

In an embodiment, the agent is administered in a therapeutically effective amount to the subject, including a human, having or suspected of having or of being susceptible to, a liver disease and/or liver condition in which oxidative stress is a contributory factor.

“Therapeutically effective amount” refers to an amount that can provide therapeutic, palliative or prophylactic relief to a subject, including a human, having or suspected of having or of being susceptible to, a liver disease and/or liver condition in which oxidative stress is a contributory factor. It will be appreciated that the therapeutically effective amount of the agent will be an amount that is capable of inhibiting the expression and/or biological activity of microRNA-144 in a subject.

The term “having or suspected of having or of being susceptible to” indicates that the subject has been determined to be, or is suspected of being, increased risk, relative to the general population of such subjects, of developing a liver disease and/or liver condition as herein defined.

For example, a subject could have a personal and/or family medical history that includes frequent occurrences of a particular disease or disorder, for example, obesity can be a contributory factor in the development of a liver disease and/or liver condition as defined herein. As another example, a subject could have had such a susceptibility determined by methods of the invention, including determining the expression and/or biological activity of miR-144.

By “a subject” we include the meaning of a patient, or individual in need of treatment and/or prevention of a disease or condition as described herein. The subject may be a vertebrate, such as a vertebrate mammal.

In an embodiment, the subject is selected from the group comprising: a primate (for example, a human; a monkey; an ape); a rodent (for example, a mouse, a rat, a hamster, a guinea pig, a gerbil, a rabbit); a canine (for example, a dog); a feline (for example, a cat); an equine (for example, a horse); a bovine (for example, a cow); and/or a porcine (for example, a pig).

Most preferably, the subject is a human subject.

The term “oxidative stress” as used herein is a disturbance in the balance between the production of reactive oxygen species ROS, also termed “free radicals” and antioxidant defences. Oxidative stress can be measured by methods known in the art, as described herein, and as described in the accompanying Examples.

Reactive oxygen species (ROS) are produced by living organisms as a result of normal cellular metabolism and environmental factors, such as air pollutants or cigarette smoke. ROS are highly reactive molecules and can damage cell structures such as carbohydrates, nucleic acids, lipids, and proteins and alter their functions. The shift in the balance between oxidants and antioxidants in favour of oxidants is termed “oxidative stress.”. Regulation of reducing and oxidizing (redox) state is critical for cell viability, activation, proliferation, and organ function. Aerobic organisms have integrated antioxidant systems, which include enzymatic and nonenzymatic antioxidants that are usually effective in blocking harmful effects of ROS. However, in pathological conditions, the antioxidant systems can be overwhelmed (Birben E., Oxidative stress and antioxidant defense, World Allergy Organ Journal, 2012, 5(1): 9-19).

By “a liver disease and/or liver condition in which oxidative stress is a contributory factor” we include the meaning of any biological or medical condition or disorder of the liver in which at least part of the pathology is mediated by oxidative stress. We also include that the primary site of the disease is the liver. The liver disease and/or liver condition may be caused by the oxidative stress or may simply be characterised by oxidative stress. Oxidative stress may contribute directly by generating products that cause pathology (e.g. ROS), and/or oxidative stress may contribute indirectly by altering expression of antioxidant response genes to cause pathology. Oxidative stress leads to DNA damage, lipid peroxidation resulting in disruption of the plasma membrane bilayer, protein fragmentation and disruption of signaling. All of these effects contribute to liver cell death, inflammation and fibrosis, hallmarks of liver diseases such as NASH and cirrhosis. It is therefore expected that reducing oxidative stress will then prevent, ameliorate or treat the condition so characterised.

Examples of particular liver diseases and/or liver conditions are described below.

Preferably, the agent decreases miR-144 expression and/or activity in cells of the liver.

By “miR-144 expression” we include the level, amount, concentration, or abundance of, miR-144. The term “expression” may also refer to the rate of change of the amount, concentration of miR-144. Expression can be represented, for example, by the amount or synthesis rate of miR-144. The term can be used to refer to an absolute amount of a miR-144 in a sample or to a relative amount of miR-144, including amount or concentration determined under steady-state or non-steady-state conditions. Expression may also refer to an assay signal that correlates with the amount, concentration, or rate of change of miR-144. The expression of miR-144 can be determined relative to the level of miR-144 in a control sample.

A decrease of the expression level of a nucleotide sequence (or steady state level of the encoded miRNA molecule, e.g. miR-144) is preferably a detectable decrease in the expression level of a nucleotide (or steady state level of an encoded miRNA molecule or any detectable change in a biological activity of miR-144) using a method as described herein as compared to the expression level of a corresponding nucleotide sequence (or steady state level of a corresponding encoded miRNA molecule or equivalent or source thereof) in a control, such as a healthy subject.

The detection of the expression of miR-144 may be carried out using any technique known in the art. The assessment of the expression level or of the presence of miR-144 is preferably performed using a suitable assay such as real time (RT) quantitative PCR (RT-qPCR), microarrays, bead arrays, in situ hybridization and/or Northern blot analysis.

Preferably, a decrease of the expression of miR-144 in cells of the liver includes a decrease of at least 10% of the expression of miR-144 in cells of the liver compared to the expression of miR-144 in cells of the liver in the absence of an inhibitor using a suitable method. More preferably, a decrease of the expression of miR-144 in cells of the liver means a decrease of at least 15%, even more preferably at least 20%, at least 30%, at least 40%, at least 50%, at least 60%, at least 70%, at least 80%, at least 90%, or 100% compared to the expression of miR-144 in cells of the liver in the absence of an inhibitor using a suitable method. In this case, there is no detectable expression of miR-144 in cells of the liver.

In an embodiment, the agent decreases the expression of miR-144 in cells of the liver by at least 2, or at least 5, or at least 10, or at least 50 fold compared to the expression of miR-144 in cells of the liver in the absence of an inhibitor using a suitable method. More preferably, the agent decreases the expression of miR-144 in cells of the liver by at least 100, or at least 1,000, or at least 10,000 fold compared to the expression of miR-144 in cells of the liver in the absence of an inhibitor using a suitable method.

By “miR-144 activity” we include the biological activity or biological action of miR-144, and this refers to any function(s) exhibited or performed by a naturally occurring, and/or wild type form of miR-144 as measured or observed in vivo (i.e. in the natural physiological environment of the protein) or in vitro (i.e. under laboratory conditions).

In an embodiment, the agent may be one that decreases the biological activity of miR-144 in cells of the liver by at least 2, or at least 5, or at least 10, or at least 50 fold compared to the biological activity of miR-144 in the absence of an inhibitor. More preferably, the agent decreases the biological activity of miR-144 in cells of the liver by at least 100, or at least 1,000, or at least 10,000 fold compared to the biological activity of miR-144 in cells of the liver in the absence of an inhibitor.

In an embodiment, a decrease of miR-144 activity is quantified using a specific assay for miR-144 activity. A preferred assay is RT-qPCR.

In a preferred embodiment, the agent is one that binds to miR-144 in order to inhibit the biological activity of miR-144. More preferably the agent is one that selectively binds to miR-144.

By an agent that “selectively binds” to miR-144, we include the meaning that the agent binds to miR-144 with a greater affinity than to an unrelated miRNA such as miR-532. Preferably, the agent binds to miR-144 with at least 5, or at least 10 or at least 50 times greater affinity than to the unrelated miRNA. More preferably, the agent binds to miR-144 with at least 100, or at least 1,000, or at least 10,000 times greater affinity than to an unrelated miRNA. Such binding may be determined by methods well known in the art, including RNA fluorescence in situ hybridization (FISH), RNA fluorescence in vivo hybridization (FIVH), surface plasma resonance (SPR), electrophoretic mobility shift assay and cross-linking, ligation, and sequencing of hybrids (CLASH).

It will be appreciated that inhibition of miR-144 which follows binding of the agent to miR-144 may be termed “direct inhibition”. An example for a direct inhibition is the interaction of a miRNA molecule with an antisense RNA (i.e. with an RNA that has a reverse complementary sequence to the miRNA molecule), thereby forming a duplex, which leads to the degradation of the miRNA molecule.

In an embodiment, the agent does not bind to miR-144 in order to inhibit the biological activity of miR-144. It will be appreciated that this may be termed “indirect inhibition”. An example of indirect inhibition is the inhibition of a protein that is involved in the transcription and/or processing of a miRNA molecule, leading to a decrease in its expression.

In one embodiment, downregulation of expression of miR-144 occurs preferentially in cells of the liver.

Preferably, the agent is delivered to cells of the liver.

By “delivered to cells of the liver”, we include that the agent is targeted to cells of the liver and will be active in cells of the liver. Preferably, the agent is selectively delivered to cells of the liver. For example, if the agent is selectively delivered to cells of the liver, cells of the liver will selectively contain the agent to a greater extent than cells of a different organ, for example, the brain, or kidney. Accordingly, following deliver of the agent to cells of the liver, miR-144 will be inhibited in cells of the liver without affecting miR-144 expression and/or activity in cells of other organs, such as the brain.

Accordingly, preferably the delivery of the agent is by local delivery. By “local delivery” we include delivery of the agent directly to a target site within an organism. For example, a compound can be locally delivered by direct injection into the liver.

Agents of the invention including but not limited to antagomirs, antisense oligonucleotides, inhibitory RNA molecules, or other modulators of miR-144 expression and/or activity may be administered by any method known to those in the art suitable for delivery to the liver, such as those described in Juliano R. L., The delivery of therapeutic oligonucleotides, Nucleic Acids Res., 2016; 44(14): 6518-6548.

In one embodiment, the presence of the agent, such as a nucleic acid agent in cells of the liver is detectable at 24, 48, 72 and/or 96 hours after administration. In one embodiment, downregulation of expression of miR-144 is detectable at 24, 48, 72 and/or 96 hours after administration.

Preferably, the cells of the liver are phagocytic liver cells, hepatocytes, endothelial cells and/or neutrophils.

The liver consists of a plurality of cell types.

Hepatocytes are polyhedral in shape and vary in size from 12 to 25 μm in diameter and contain one or sometimes two distinct nuclei in each cell. Hepatocytes comprise 60%-80% of all liver cells, and they conduct the metabolic, bio-synthetic, detoxification and biliary secretory functions of the liver.

The sinusoids are made of endothelial cells, phagocytic Kupffer cells, stellate cells (Ito cells), and pit cells.

Liver macrophages or Kupffer cells are responsible for detoxifying the liver by clearing pathogens such as bacteria and dead cells. They also contribute to the formation of bile acids (Jager J., Liver innate immune cells and insulin resistance: the multiple facets of Kupffer cells., J Intern Med. 2016 August; 280(2):209-20). They can also directly regulate insulin signalling in hepatocytes (Morgantini C., Liver macrophages regulate systemic metabolism through non-inflammatory factors, 2019, Nature Metabolism 1, 445-459)

Endothelial cells of the liver include liver sinusoidal endothelial cells (LSEC) which are the most abundant non-parenchymal cells in the liver. LSEC are highly specialized and unique from vascular endothelial cells as they lack a basement membrane and have a multitude of fenestrae that regulate transport of macromolecules, including lipids and lipoproteins, across the sinusoid.

Neutrophils (also known as neutrocytes) are the most abundant type of granulocytes and the most abundant (60% to 70%) type of white blood cells in most mammals. They form an essential part of the innate immune system. Neutrophils are a type of phagocyte and are normally found in the bloodstream. During the beginning (acute) phase of inflammation, neutrophils are one of the first-responders of inflammatory cells to migrate towards the site of inflammation. They migrate through the blood vessels in a process called chemotaxis. It has been shown that inappropriate activation and homing of neutrophils to the microvasculature contributes to the pathological manifestations of many types of liver disease, such as viral hepatitis, non-alcoholic fatty liver disease, liver fibrosis and cirrhosis. (Xu R et al, The role of neutrophils in the development of liver diseases, Cell Mol Immunol. 2014 May; 11(3): 224-231).

Dendritic cells (DCs) are antigen-presenting cells (also known as accessory cells) of the mammalian immune system. Their main function is to process antigen material and present it on the cell surface to the T cells of the immune system. They act as messengers between the innate and the adaptive immune systems. DCs in the liver are uniquely positioned to monitor the portal circulation, and they are crucial in the regulation of responses to blood-borne pathogens, hepatic immune tolerance, liver homeostasis and fibrosis. (Rahman A., Dendritic Cells and Liver Fibrosis, Biochim Biophys Acta. 2013 July; 1832(7): 998-1004)

In an embodiment, the phagocytic liver cells are liver macrophages (LMs).

In an embodiment, delivering the agent to cells of the liver results in decreased miR-144 expression and/or activity in cells of the liver, for example in phagocytes, hepatocytes, endothelial cells and/or neutrophils.

As can be seen in the accompanying Examples, using Glucan encapsulated RNAi Particle (GeRP) technology, which delivers siRNA and silences genes specifically in LMs without affecting gene expression in other cells of the liver or the rest of the body, the inventors found that selective silencing of miR-144 in LMs was sufficient to decrease ROS release by LMs and hepatocytes and eventually accumulation in the whole liver through the rescue of NRF2 in obese mice. This latter result was surprising since GeRPs cannot be delivered to non-phagocytic cells such as hepatocytes but suggested as crosstalk between LMs and hepatocytes. As can be seen from the accompanying Examples, selective knockdown of miR-144 in LMs leads to a reduction in miR-144 transcription in hepatocytes.

Without being bound by theory, the inventors hypothesise that targeting miR-144 in all cells of the liver would be beneficial. For example, a prominent feature of the inflammation observed in NASH is neutrophil accumulation, and liver neutrophil dysfunction has been described in relation to several liver diseases, including non-alcoholic fatty liver disease, alcoholic liver disease, liver cirrhosis, liver failure and hepatocellular carcinoma (Xu R et al., Cell Mol Immunol. 2014 May; 11(3):224-31). Additionally, endothelial cells have been shown to contribute to oxidative stress in NAFLD/NASH (Matsumoto M et al., Free Radic Biol Med. 2018 Feb. 1; 115:412-420; and Peters K M et al., Curr Opin Lipidol. 2018 October; 29(5):417-422).

In an embodiment, oxidative stress is induced by obesity, alcohol, environmental pollutants, and/or drugs, such as anti-inflammatory drugs, anti-analgesic drugs, anti-cancer drugs and/or antidepressants.

In an embodiment, oxidative stress in the liver is induced by obesity. It will be understood that a subject is classified as obese if they have a body mass index over 30. Fatty liver is the result of excessive lipid accumulation due to a lower fat storage capacity of adipose tissue in obesity-associated insulin resistance. The inability of the liver to handle this overload of fat leads to aberrant lipid peroxidation, excessive production of Reactive Oxygen Species (ROS) and oxidative stress.

In an embodiment, oxidative stress in the liver is induced by alcohol. Excessive generation of free radicals is believed to play a central role in many pathways of alcohol-induced damage. Free radicals can result in oxidative stress, which is characterized by a disturbance in the balance between free radical generation and free radical scavenging, including repair of damaged molecules. A free radical is a cluster of atoms containing at least one unpaired electron. Therefore, it is known that alcohol induces oxidative stress (Wu D. et al, J Gastroenterol Hepatol. 2006 October; 21 Suppl 3:S26-9).

In an embodiment, oxidative stress is induced by environmental pollutants. Environmental pollutants such as mercury increases the intracellular levels of reactive oxygen species and induces oxidative stress, resulting in tissue damaging effects, since the toxicity of this metal is associated with superoxide generation and glutathione (GSH) depletion (Bando I et al., J Biochem Mol Toxicol. 2005; 19(3):154-61).

In an embodiment, oxidative stress in the liver is induced by drugs including anti-inflammatory drugs, anti-analgesic drugs, anti-cancer drugs and/or antidepressants. Several types of drugs including anti-inflammatory drugs, anti-analgesic drugs, anti-cancer drugs and/or antidepressants such as Sulfasalazine, Zoledronic acid, Paracetamol, Morphine, Doxorubicin, paclitaxel and docetaxel, Nimesulide, Fluoxetine/clozapine and Isoniazid have all been implicated in inducing oxidative stress (Linares V et al., Toxicology. 2009 Feb. 27; 256(3):152-6; Karabulut A B et al., Transplant Proc. 2010 November; 42(9):3820-2; Mladenović D et al., Food Chem Toxicol. 2009 April; 47(4):866-70; Samarghandian S et al., Int J Clin Exp Med. 2014 May 15; 7(5):1449-53; Pieniążek A et al., Adv Med Sci. 2013; 58(1):104-11; Kale V M et al., Chem Res Toxicol. 2010 May 17; 23(5):967-76; Zlatković J et al., Eur J Pharm Sci. 2014 Aug. 1; 59:20-30; Shuhendler A J et al., Nat Biotechnol. 2014 April; 32(4):373-80).

Preferably, the oxidative stress is oxidative stress in cells of the liver.

Preferred cells of the liver include those described above, namely phagocytes (such as macrophages), hepatocytes, endothelial cells and neutrophils.

Therefore, in an embodiment, oxidative stress in cells of the liver is induced by obesity, alcohol, environmental pollutants, and/or drugs, such as anti-inflammatory drugs, anti-analgesic drugs, anti-cancer drugs and/or antidepressants.

In an embodiment, the oxidative stress in the liver is characterised by at least one of:
a) increased lipid peroxidation;
b) Reactive Oxygen Species (ROS) increase and/or accumulation;
c) decreased Nuclear Factor Erythroid 2-Related Factor 2 (NRF2) activity and/or protein levels; and/or
d) increased expression and/or activity of miR-144.

In an embodiment, the oxidative stress in the liver is characterised by increased lipid peroxidation. Increased lipid peroxidation is a common marker of oxidative stress, wherein for example. By “lipid peroxidation” we include a process under which oxidants such as free radicals attack lipids containing carbon-carbon double bond(s), especially polyunsaturated fatty acids. Lipid peroxidation can be measured by methods known in the art, and as described in the accompanying Examples, for example by the measurement of Malondialdehyde (MDA), a reactive aldehyde produced during lipid peroxidation (see for example Assay Kit from Abcam; ab118970)). It will be appreciated that if lipid peroxidation is increased in a test sample, for example from an obese subject, compared to a control sample, such as from a healthy and/or lean subject, this could indicate the presence of oxidative stress.

In an embodiment, lipid peroxidation is increased by at least 2, or at least 5, or at least 10, or at least 50 fold compared to lipid peroxidation in a control subject, such as a lean and/or healthy subject. Preferably, lipid peroxidation is increased by at least 100, or at least 1,000, or at least 10,000 fold compared to lipid peroxidation in a control subject, such as a lean and/or healthy subject.

In an embodiment, the oxidative stress in the liver is characterised by reactive oxygen species increase and/or accumulation. By “Reactive oxygen species (ROS)”, also termed “free radicals” and “oxygen radical” we include a type of unstable molecule that contains oxygen and that easily reacts with other molecules in a cell. Accumulation of ROS in cells may cause damage to DNA, RNA, and proteins, and may cause cell death. ROS increase and/or accumulation can be measured by methods known in the art, and as described in the accompanying Example, for example intracellular ROS can be measured by OxiSelect™ In Vitro ROS/RNS Assay Kit (NordicBiosite; STA-347) and extracellular ROS can be measured by Amplex™ Red Hydrogen Peroxide/Peroxidase Assay Kit (ThermoFisher; A22188). It will be appreciated that if ROS levels are increased in a test sample, for example from an obese subject, compared to a control sample, such as from a healthy and/or lean subject, this could indicate the presence of oxidative stress.

In an embodiment, ROS is increased in a subject by at least 2, or at least 5, or at least 10, or at least 50 fold compared to ROS in a control subject, such as a lean and/or healthy subject. Preferably, ROS is increased by at least 100, or at least 1,000, or at least 10,000 fold compared to ROS in a control subject, such as a lean and/or healthy subject.

In an embodiment, the oxidative stress in the liver is characterised by decreased Nuclear Factor Erythroid 2-Related Factor 2 (NRF2) activity and/or protein levels. As described above NRF2, also termed “NFE2L2” and “Nrf2”, is a basic leucine zipper transcription factor, is a master regulator of redox homeostasis. Under normal physiological conditions, NRF2 is targeted to proteasomal degradation through its association with Kelch-like ECH-associated protein-1 (KEAP1). Conversely, upon oxidative stress this complex dissociates and NRF2 translocates to the nucleus where it binds to the antioxidant responsive element (ARE), thus driving the antioxidant response. Methods for measuring the protein level of NRF2 include methods known in the art, and as described in the accompanying Examples, for example by western blot using an NRF2 antibody (Abcam; ab62352), or using Chromatin Immunoprecipitation (ChIP) using an antibody against NRF2 to check its binding to specific antioxidant responsive elements (ARE) DNA regions. NRF2 is a transcription factor and its activity can be analysed by measuring the expression of its target genes (including but not limited to Nqo1, Hmox1, Ces2g and Gstp1) by RTqPCR. If NRF2 is active, the expression of its target genes is increased. It will be appreciated that if NRF2 activity and/or expression is decreased in a test sample, for example from an obese subject, compared to a control sample, such as from a healthy and/or lean subject, this could indicate the presence of oxidative stress.

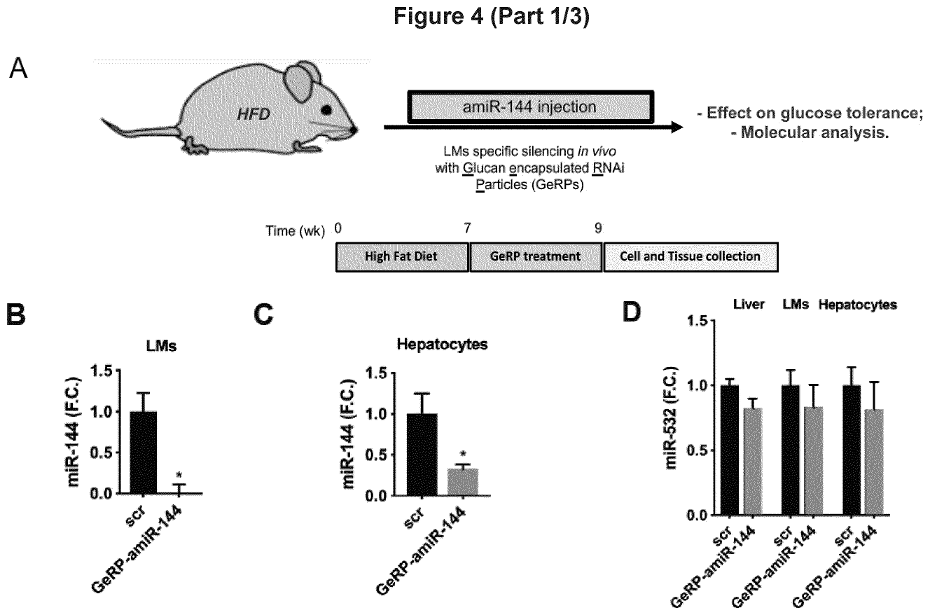

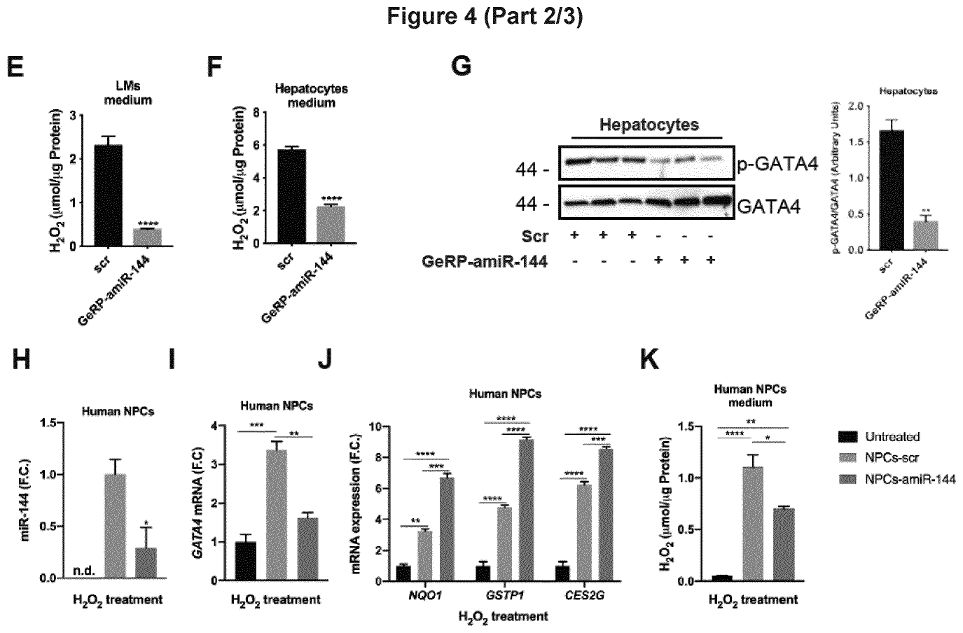

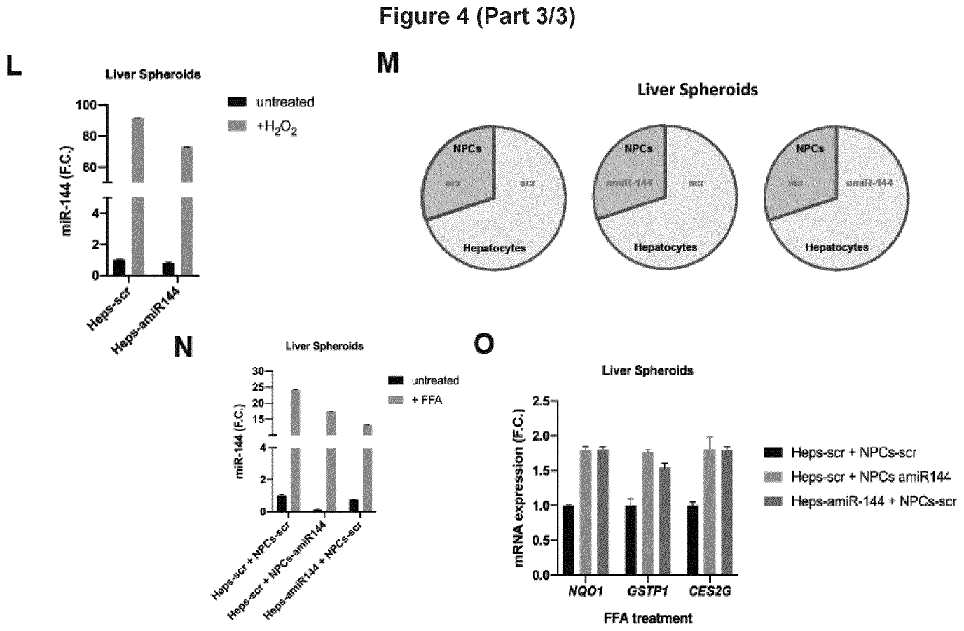

Healthy cells treated with H₂O₂, which induces oxidative stress and the anti-oxidant response/NRF2 activation could also be used as control. Since healthy cells have a normal anti-oxidant response, NRF2 will be activated in presence of H₂O₂ (see FIG. 4H-K which shows that human liver spheroids or human non-parenchymal cells treated with H₂O₂ display an increased expression of the NRF2 target genes as measured by RTqPCR).

As can be seen from the accompanying Examples, the inventors observed a decrease in NRF2 protein levels in liver macrophages, hepatocytes and the whole liver in a model of induced obesity. However, surprisingly NRF2 mRNA levels and transcription remained unchanged in a model of induced obesity.

In an embodiment, NRF2 activity and/or protein levels is decreased in a subject by at least 2, or at least 5, or at least 10, or at least 50 fold compared to NRF2 activity and/or protein levels in a control subject, such as a lean and/or healthy subject. Preferably, NRF2 activity and/or protein levels is decreased by at least 100, or at least 1,000, or at least 10,000 fold compared to NRF2 activity and/or protein levels in a control subject, such as a lean and/or healthy subject. By “NRF2 protein levels” we include the expression, amount, concentration, or abundance of, NRF2. The term “levels” may also refer to the rate of change of the amount, concentration of NRF2. Expression can be represented, for example, by the amount or synthesis rate of NRF2 protein. The term can be used to refer to an absolute amount of a NRF2 in a sample or to a relative amount of NRF2, including amount or concentration determined under steady-state or non-steady-state conditions. NRF2 protein levels can be determined relative to the level of NRF2 in a control sample.

In an embodiment, the oxidative stress in the liver is characterised by increased expression and/or activity of miR-144. The term “expression” is as defined herein. Methods for measuring the expression of miR-144 include methods known in the art, and as described in the accompanying Examples, for example by using RT-qPCR. Methods for measuring the activity of miR-144 include those described herein. It will be appreciated that if miR-144 activity and/or expression is increased in a test sample, for example from an obese subject, compared to a control sample, such as from a healthy and/or lean subject, this could indicate the presence of oxidative stress.

In an embodiment, expression and/or activity of miR-144 is increased in a subject by at least 2, or at least 5, or at least 10, or at least 50 fold compared to expression and/or activity of miR-144 in a control subject, such as a lean and/or healthy subject. Preferably, expression and/or activity of miR-144 is increased by at least 100, or at least 1,000, or at least 10,000 fold compared to ROS in a control subject, such as a lean and/or healthy subject.

As described in the accompanying Examples, miR-144 expression is mediated by GATA4. Without being bound by theory the inventors hypothesise that ROS act as a secondary messenger to activate ERK and GATA4 leading to the increased expression of miR-144. Therefore, it will be appreciated that measuring activation of ERK and GATA4 could be an indication that miR-144 expression is increased. GATA4 and ERK activation can be measured by methods known in the art, and as described in the Examples.

In an embodiment, oxidative stress is characterised by any of (b)-(d).

As described in the accompanying Examples, NRF2 protein levels are reduced when miR-144 is induced since NRF2 is a direct target of miR-144. Therefore, it will be appreciated that NRF2 does not need to be measured if miR-144 levels are high and ROS levels are increased. In addition, miR-144 levels are increased by oxidative stress, for example by excessive ROS accumulation and/or production, therefore, an increase in miR-144 could reflect liver oxidative stress.

In an embodiment, the liver disease and/or liver condition in which oxidative stress is a contributory factor is selected from the group comprising: non-alcoholic steatohepatitis (NASH), non-alcoholic fatty liver disease (NAFLD), fibrosis, cirrhosis, hepatocellular carcinoma (HCC), and/or liver damage induced by alcohol, environmental pollutants, and/or drugs, such as anti-inflammatory drugs, anti-analgesic drugs, anti-cancer drugs and/or antidepressants.

In an embodiment, the subject displays at least one of insulin resistance, and obesity.

By “insulin resistance” we include the inability of insulin-target tissues to respond to insulin. In adipose tissue, insulin is unable to induce glucose uptake and lipid storage, and to block lipid release; in muscle, insulin is unable to induce glucose uptake; and in liver, insulin is unable to block hepatic glucose production. Insulin sensitivity can be assessed by methods known in the art, such as by homeostatic model assessment (HOMA-IR), as described in the Examples. In general, insulin resistance can also be diagnosed by any of the following measurements:
Values over 2 following an assessment using HOMA-IR
Glucose values 2 hours during an oral glucose tolerance test: 140 to 199 mg/dL
HbA1c circulating levels: 5.7 to 6.4 percent

The combination of all these measurements are usually used in the clinic.

By “non-alcoholic fatty liver disease (NAFLD)” we include a disease state characterised by the build-up of fat in the liver, generally observed in obese individuals. We include a condition characterized by fatty inflammation of the liver that is not due to excessive alcohol use (for example, alcohol consumption of over 20 g/day). In certain embodiments, NAFLD is related to insulin resistance and the metabolic syndrome.

By “non-alcoholic steatohepatitis (NASH)” we include a condition characterised by inflammation and the accumulation of fat and fibrous tissue in the liver, that is not due to excessive alcohol use. NASH is a common liver disease that is characterized histologically by hepatic steatosis, lobular inflammation, and hepatocellular ballooning; it can progress to cirrhosis in up to 15% of patients. There is currently no therapy that is of proven benefit for NASH. The disease is closely associated with insulin resistance and features of the metabolic syndrome such as obesity, hypertriglyceridemia, and type 2 diabetes (Sanyal A J et al., 2010). NASH is an extreme form of NAFLD.

Although dysregulated lipid accumulation occurs across the non-alcoholic fatty liver disease spectrum, the features of liver cell injury, such as hepatocyte ballooning, cytoskeletal changes (Mallory-Denk bodies) and hepatocyte apoptosis, predominantly occur in NASH and distinguish NASH from simple steatosis.

By “fibrosis” we include the excessive accumulation of extracellular matrix proteins including collagen in the liver.

By “cirrhosis” we include the late stage of scarring (fibrosis) of the liver.

By “hepatocellular carcinoma (HCC)” we include a primary malignancy of the liver which occurs predominantly in patients with underlying chronic liver disease and cirrhosis. HCC is now the third leading cause of cancer deaths worldwide, with over 500,000 people affected. HCC is refractory to most cancer drugs. Methods for diagnosing HOC include ultrasound, imaging (CT scan and MRI), but the most accurate is pathology following a liver biopsy. Current treatment options for HCC include liver transplantation, surgery to remove the cancer, chemotherapy and/or radiotherapy.

Liver conditions such as NASH, NAFLD, fibrosis and cirrhosis may be diagnosed by those skilled in the art and may include a review of medical history, a physical exam, and various tests. Medical history can be reviewed for risk factors such as weight/obesity, insulin resistance, high levels of triglycerides or abnormal levels of cholesterol in your blood, metabolic syndrome and/or type 2 diabetes. Physical symptoms including an enlarged liver, signs of insulin resistance such as darkened skin patches over your knuckles, elbows, and knees and/or signs of cirrhosis, such as jaundice, a condition that causes your skin and whites of your eyes to turn yellow can indicate liver disease. Other methods for diagnosing liver disease include a blood test for liver enzymes alanine aminotransferase (ALT) and/or aspartate aminotransferase (AST). Other methods for diagnosing liver disease include imaging tests including abdominal ultrasound, magnetic resonance imaging (MRI), transient elastography, computed tomography (CT), ultrasound elastography, MR elastography (MRE).

None of the above are particularly accurate and the diagnosis of non-alcoholic steatohepatitis (NASH) is defined by the presence and pattern of specific histological abnormalities on liver biopsy. The histology and scoring by pathologists looking at fat, necro-inflammation, and fibrosis. NASH (yes or no), Fibrosis (F0 to F4, F0 being simple fat accumulation and F4 severe fibrosis/cirrhosis). A separate system of scoring the features of nonalcoholic fatty liver disease (NA) called the NAFLD Activity Score (NAS) is well known in the art and was developed as a tool to measure changes in NAFLD during therapeutic trials. However, some studies have used threshold values of the NAS, specifically NAS 5, as a surrogate for the histologic diagnosis of NASH (i.e. in the absence of a liver biopsy).

Currently treatment for liver disease varies depending on the cause. A physician will typically recommend treatment aimed at preventing or delaying progression of fibrosis such as dietary changes, anti-inflammatory medications and medications for insulin resistance, cholesterol and diabetes management, exercise and weight loss and/or eliminating alcohol use.

In the context of the present invention, “preventing” a liver disease and/or liver condition, may also include preventing progression of NAFLD or NASH (e.g. preventing progression to fibrosis and/or cancer). In the context of the present invention, prevention also includes upregulating the antioxidant response in the liver or the subject. By prevention we also include preventing the development of resistance to treatment and/or therapy. For example, resistance may be prevented through the simultaneous administration of more than one therapy/drug (combination therapy) as described herein.

Preferably, miR-144 mediates at least one of the following in cells of the liver:
i. NRF2 activity and/or protein levels;
ii. production of extracellular ROS;
iii. GATA4 phosphorylation and/or activity;
iv. levels of intracellular glycogen; and
v. endogenous antioxidant response.

By “mediates” we include the meaning that miR-144 expression and/or activity is responsible for, or regulates, NRF2 activity and/or protein levels; production of extracellular ROS; GATA4 phosphorylation and/or activity; levels of intracellular glycogen; and/or endogenous antioxidant response.

GATA4 phosphorylation can be measured by methods such as those described in the accompanying Examples and by methods known in the art, including but not limited to western blot and Chromatin Immunoprecipitation (ChIP) using an antibody against GATA4 to measure its binding to DNA.

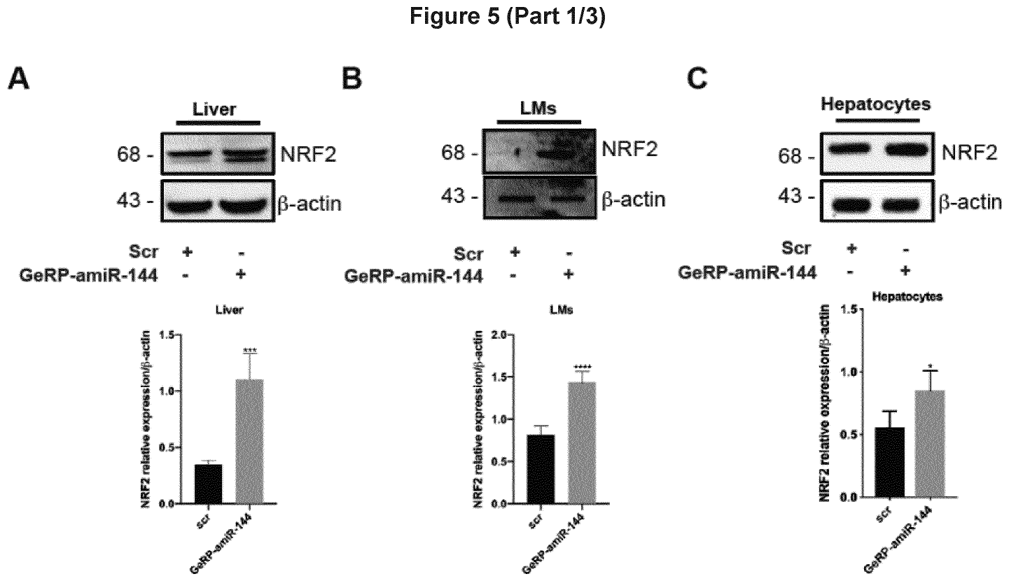

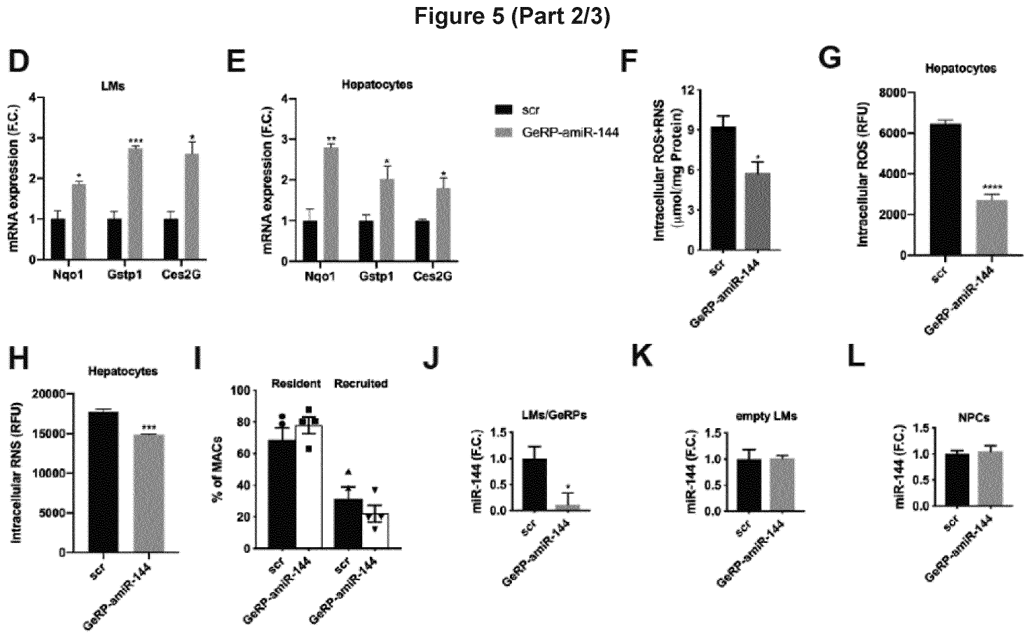

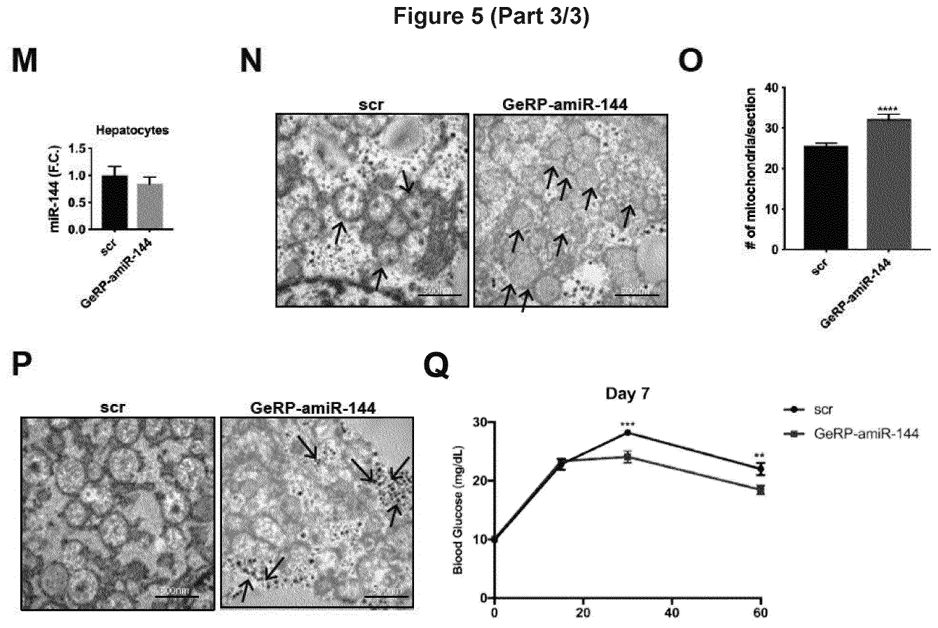

Levels of intracellular glycogen can be measured by methods known in the art, including but not limited to using a Glycogen Assay Kit (Abcam; ab65620). As described in the accompanying Example, the inventors observed increased levels of stored intracellular glycogen in the liver of mice treated with an agent which inhibits miR-144 (FIG. 5M). Consistent with the increased glycogen stores, glucose tolerance tests in mice treated with an agent which inhibits miR-144 showed an improved glucose homeostasis compared to control mice (FIG. 5N).

As used herein “endogenous antioxidant response” includes the natural response of the cell to oxidative stress without the addition of (exogenous) anti-oxidants. NRF2 drives the endogenous response as it is produced by the cells themselves and induces the expression of genes encoding proteins able to scavenge ROS. The endogenous antioxidant response glycogen can be measured by methods known in the art, including but not limited to the measurement of NRF2 activity and protein levels, and ROS levels.

Preferably, decreased expression and/or activity of miR-144 causes at least one of the following in cells of the liver:
i. increased NRF2 activity and/or protein levels;
ii. decreased intracellular ROS and/or decreased release of ROS;
iii. decreased phosphorylation and/or activity of GATA4;
iv. increased levels of intracellular glycogen; and
v. restored and/or increased endogenous antioxidant response.

In an embodiment, decreased expression and/or activity of miR-144 causes improved glucose homeostasis.

Each of the parameters above can be measured in a subject following administration of an agent which inhibits miR-144.

By “increased NRF2 activity and/or protein levels” we include that following administration of an agent which inhibits miR-144, NRF2 activity and/or protein levels are increased in a subject who has a liver disease and/or liver condition in which oxidative stress is a contributory factor compared to a control subject who has a liver disease and/or liver condition in which oxidative stress is a contributory factor, who was not administered the agent, or who was administered a placebo. In an embodiment, a placebo is a scrambled control oligonucleotide, such as that described in the Examples. Alternatively, the control could be a control sample which comprises healthy cells treated with H₂O₂, which induces oxidative stress, but not treated with the agent which inhibits miR-144.

In an embodiment, NRF2 activity and/or protein level is increased by at least 2, or at least 5, or at least 10, or at least 50 fold compared to the NRF2 activity and/or protein levels in the absence of an inhibitor. Preferably, NRF2 activity and/or protein level is increased by at least 100, or at least 1,000, or at least 10,000 fold compared to the NRF2 activity and/or protein levels in the absence of an inhibitor.

Without being bound by theory, because miRNAs are known to regulate both transcription and translation, and expression of miR-144 is increased in liver cells from obese subjects which correlated with a decrease in the protein levels of NRF2, the inventors hypothesise that miR-144 target the translation of NRF2. Accordingly, silencing, or downregulation of miR-144 causes an increase in NRF2 activity and/or protein levels.

By “decrease of intracellular ROS and/or decreased release of ROS” we include that following administration of an agent which inhibits miR-144, intracellular ROS and/or release of ROS is reduced in a subject who has a liver disease and/or liver condition in which oxidative stress is a contributory factor compared to a control subject who has a liver disease and/or liver condition in which oxidative stress is a contributory factor, who was not administered the agent, or who was administered a placebo.

In an embodiment, intracellular ROS and/or release of ROS is decreased by at least 2, or at least 5, or at least 10, or at least 50 fold compared to the intracellular ROS and/or release of ROS in the absence of an inhibitor. Preferably, intracellular ROS and/or release of ROS is decreased by at least 100, or at least 1,000, or at least 10,000 fold compared to the intracellular ROS and/or release of ROS in the absence of an inhibitor.

By “decreased phosphorylation and/or activity of GATA4” we include that following administration of an agent which inhibits miR-144, GATA4 activity and/or phosphorylation is reduced in a subject who has a liver disease and/or liver condition in which oxidative stress is a contributory factor compared to a control subject who has a liver disease and/or liver condition in which oxidative stress is a contributory factor, who was not administered the agent, or who was administered a placebo. As can be seen from the accompanying Examples, GATA4 phosphorylation was reduced in hepatocytes upon silencing of the miR-144 in LMs.

In an embodiment, GATA4 activity and/or phosphorylation is decreased by at least 2, or at least 5, or at least 10, or at least 50 fold compared to the GATA4 activity and/or phosphorylation in the absence of an inhibitor. Preferably, GATA4 activity and/or phosphorylation is decreased by at least 100, or at least 1,000, or at least 10,000 fold compared to the GATA4 activity and/or phosphorylation levels in the absence of an inhibitor.

By “increased levels of intracellular glycogen” we include that following administration of an agent which inhibits miR-144, levels of intracellular glycogen are increased in a subject who has a liver disease and/or liver condition in which oxidative stress is a contributory factor compared to a control subject who has a liver disease and/or liver condition in which oxidative stress is a contributory factor, who was not administered the agent, or who was administered a placebo.

In an embodiment, levels of intracellular glycogen are increased by at least 2, or at least 5, or at least 10, or at least 50 fold compared to the levels of intracellular glycogen in the absence of an inhibitor. Preferably, levels of intracellular glycogen are increased by at least 100, or at least 1,000, or at least 10,000 fold compared to the levels of intracellular glycogen in the absence of an inhibitor.

By “restored endogenous antioxidant response” we include that following administration of an agent which inhibits miR-144, the endogenous antioxidant response is restored in a subject who has a liver disease and/or liver condition in which oxidative stress is a contributory factor to substantially similar levels as observed in a control subject who does not have a liver disease and/or liver condition in which oxidative stress is a contributory factor, and who was not administered the agent.

By “increased endogenous antioxidant response” we include that following administration of an agent which inhibits miR-144, the endogenous antioxidant response is increased in a subject who has a liver disease and/or liver condition in which oxidative stress is a contributory factor compared to a control subject who has a liver disease and/or liver condition in which oxidative stress is a contributory factor, who was not administered the agent, or who was administered a placebo.

In an embodiment, the endogenous antioxidant response is increased by at least 2, or at least 5, or at least 10, or at least 50 fold compared to the endogenous antioxidant response in the absence of an inhibitor. Preferably, the endogenous antioxidant response is increased by at least 100, or at least 1,000, or at least 10,000 fold compared to the endogenous antioxidant response in the absence of an inhibitor. As described above, the endogenous antioxidant response can be measured by methods known in the art, including but not limited to the measurement of NRF2 activity and protein levels, and ROS levels.

In an embodiment, increased NRF2 activity and/or protein levels, reduction of intracellular ROS and/or reduction of release of ROS, reduced phosphorylation and/or activity of GATA4 and/or restored endogenous antioxidant response occurs in hepatocytes and/or liver macrophages. In an embodiment, increased levels of intracellular glycogen occurs in hepatocytes. In an embodiment, reduction of intracellular ROS and/or reduction of release of ROS occurs in endothelial cells and/or in neutrophils.

Preferably, the agent is selected from the group comprising: a nucleic acid molecule, and a small molecule

By a “nucleic acid” also termed “oligonucleotide”, “nucleic acid sequence,” “nucleic acid molecule,” and “polynucleotide” we include a DNA sequence or analog thereof, or an RNA sequence or analog thereof. Nucleic acids are formed from nucleotides. By “nucleotide” we include a glycosomine comprising a nucleobase and a sugar having a phosphate group covalently linked to the sugar. Nucleotides may be modified with any of a variety of substituents.

In some embodiments, the nucleic acid agent is modified, for example, to further stabilize against nucleolytic degradation. Exemplary modifications include a nucleotide base or modification of a sugar moiety. The nucleic acid agent can include modified linker agent such as a phosphorothioate in at least the first, second, or third internucleotide linkage at the 5′ or 3′ end of the nucleotide sequence. In one embodiment, the nucleic acid agent includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-0-DMAP), 2′-O-dimethylaminoethyloxyethyl (T-0-DMAEOE), or 2′-O—N-methylacetamido (2′-0-NMA). In a particularly preferred embodiment, the nucleic acid agent includes at least one 2′-O-methyl-modified nucleotide, and in some embodiments, all of the nucleotides of the nucleic acid agent include a 2′-O-methyl modification. In some embodiments, the sugar moiety of the nucleic acid can be replaced, for example, with a non-sugar moiety such as a PNA.

Teachings regarding the synthesis of particular modified oligonucleotides may be found in Beaucage, Serge L. “Synthesis of Modified Oligonucleotides and Conjugates.” Current Protocols in Nucleic Acid Chemistry 20(1): 4.0.1-4.0.4. (2005).

In an embodiment, the nucleic acid agent may be an aptamer. Aptamers can be considered chemical antibodies having the properties of nucleotide-based therapies. Aptamers are small nucleic acid molecules that bind specifically to molecular targets such as proteins. Unlike nucleic acid therapeutics that act by hybridizing to another nucleic acid target, aptamers form three-dimensional shapes that allow for specific binding to enzymes, growth factors, receptors, viral proteins, and immunoglobulins. A nucleic acid aptamer generally includes a primary nucleotide sequence that allows the aptamer to form a secondary structure (e. g., by forming stem loop structures) that allows the aptamer to bind to its target. In the context of the present invention, aptamers can include DNA, RNA, nucleic acid analogues (e. g., peptide nucleic acids), locked nucleic acids, chemically modified nucleic acids, or combinations thereof. Aptamers can be designed for a given ligand by various procedures known in the art. Aptamers can also be used to deliver the agent of the invention (Zhou J., Aptamer-targeted cell-specific RNA interference, Silenc, 2010, 1:4).

In an embodiment, the agent is a small molecule, including but not limited to small synthetic organic molecules which can directly bind to miR-144. Their molecule weight usually is less than 800 Da and they possess properties, including good solubility, bioavailability, PK/PD, metabolism, etc. A small molecule inhibitor can be designed to target miRNA at one of at least three different stages; they can interfere with primary RNA transcription, they can inhibit pre-miRNA processes by DICER and RISC, or they can inhibit the RISC and target mRNA interaction. Teachings regarding the synthesis of particular modified oligonucleotides may be found in Wen D., Small Molecules Targeting MicroRNA for Cancer Therapy: Promises and Obstacles J Control Release. 2015 Dec. 10; 219: 237-247.

The term “small molecule” includes small organic molecules, drugs, prodrugs and/or compounds. Suitable small molecules may be identified by methods such as screening large libraries of compounds (Beck-Sickinger & Weber (2001) Combinational Strategies in Biology and Chemistry (John Wiley & Sons, Chichester, Sussex); by structure-activity relationship by nuclear magnetic resonance (Shuker et al (1996) “Discovering high-affinity ligands for proteins: SAR by NMR. Science 274: 1531-1534); encoded self-assembling chemical libraries Melkko et al (2004) “Encoded self-assembling chemical libraries.” Nature Biotechnol. 22: 568-574); DNA-templated chemistry (Gartner et al (2004) “DNA-templated organic synthesis and selection of a library of macrocycles. Science 305: 1601-1605); dynamic combinatorial chemistry (Ramstrom & Lehn (2002) “Drug discovery by dynamic combinatorial libraries.” Nature Rev. Drug Discov. 1: 26-36); tethering (Arkin & Wells (2004) “Small-molecule inhibitors of protein-protein interactions: progressing towards the dream. Nature Rev. Drug Discov. 3: 301-317); and speed screen (Muckenschnabel et al (2004) “SpeedScreen: label-free liquid chromatography-mass spectrometry-based high-throughput screening for the discovery of orphan protein ligands.” Anal. Biochem. 324: 241-249). Typically, small organic molecules will have a dissociation constant for the polypeptide in the nanomolar range, particularly for antigens with cavities. The benefits of most small organic molecule binders include their ease of manufacture, lack of immunogenicity, tissue distribution properties, chemical modification strategies and oral bioavailability. Small molecules with molecular weights of less than 5000 daltons are preferred, for example less than 400, 3000, 2000, or 1000 daltons, or less than 500 daltons.

By small molecule, we also include the meaning of prodrugs thereof. For example, the agent may be administered as a prodrug which is metabolised or otherwise converted into its active form once inside the body of a subject. The term “prodrug” as used herein refers to a precursor or derivative form of a pharmaceutically active substance that is less active compared to the parent drug and is capable of being enzymatically activated or converted into the more active parent form (see, for example, D. E. V. Wilman “Prodrugs in Cancer Chemotherapy” Biochemical Society Transactions 14, 375-382 (615th Meeting, Belfast 1986) and V. J. Stella et al. “Prodrugs: A Chemical Approach to Targeted Drug Delivery” Directed Drug Delivery R. Borchardt et al (ed.) pages 247-267 (Humana Press 1985)).

Preferably, the agent is a nucleic acid molecule selected from the group comprising: an antisense oligonucleotide and an inhibitory RNA molecule.

By “RNA” we include a molecule comprising at least one ribonucleotide residue. By “ribonucleotide” we include a nucleotide with a hydroxyl group at the 2′ position of a β-D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material, such as to the end(s) of an siRNA or internally, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs may be referred to as analogs or analogs of a naturally occurring RNA.

Examples of antisense oligonucleotides include, but are not limited to, antagomirs, synthetic peptide nucleic acids (PNAs), LNA/DNA copolymers, and gapmers.

Inhibition of miRNA function may be achieved by administering antisense oligonucleotides targeting the miR-144 sequence. An antisense oligonucleotide acts through the formation of a miRNA-antisense oligonucleotide duplex through Watson-Crick binding, leading to inactivation of the miRNA either through inhibition of binding to the target mRNA or through degradation via recruitment of RNase H. Antisense oligonucleotide with higher affinity to miR-144 or with higher abundance than the mRNA target will prevent the functional effect of miR-144.

Methods of producing antisense oligonucleotides are well-known in the art and can be readily adapted to produce an antisense oligonucleotide that targets any polynucleotide sequence, see for example Joana Filipa Lima et al. (2018) Anti-miRNA oligonucleotides: A comprehensive guide for design, RNA Biology, 15:3, 338-352. The selection of antisense oligonucleotide sequences specific for a given target (i.e. miR1-44) sequence is based upon analysis of the chosen target sequence and determination of secondary structure, Tm, binding energy, and relative stability. Antisense oligonucleotides may be selected based upon their relative inability to form dimers, hairpins, or other secondary structures that would reduce or prohibit specific binding to the target mRNA in a host cell. Highly preferred target regions of the mRNA include those regions at or near the AUG translation initiation codon and those sequences that are substantially complementary to 5′ regions of the mRNA. These secondary structure analyses and target site selection considerations can be performed, for example, using v.4 of the OLIGO primer analysis software (Molecular Biology Insights) and/or the BLASTN 2.0.5 algorithm software (Altschul et al., Nucleic Acids Res. 1997, 25(17):3389-402).

Accordingly, in a preferred embodiment, the agent is an antagonistic antisense oligonucleotide. The antisense oligonucleotides may comprise ribonucleotides or deoxyribonucleotides. In some embodiments, the antisense molecule could be a single or a double stranded sequence. It will be appreciated that single-stranded antisense oligonucleotides can be applied to intercept and degrade mature miRNAs.

Both cholesterol conjugation and modification of the phosphate backbone with phosphorothioate (PS) linkages have been utilized to enhance in vivo delivery of antisense oligonucleotides. The 3′ cholesterol-conjugated, 2′-O-Me-modified antagomirs have become a well-validated experimental tool for in vivo inhibition of miRNAs (van Rooij E et al. EMBO Mol Med. 2014 July; 6(7):851-64).

Preferably, the antisense oligonucleotides have at least one chemical modification. Standard chemical modifications are known to the skilled person and include 2′-O-methyl or methoxyethyl nucleotides, 2′-F nucleotides and phosphorothioate backbone modified oligonucleotides, all of which have been shown to successfully interfere with miRNA effects.

Exemplary modifications include a nucleotide base or modification of a sugar moiety. Antisense oligonucleotides may be comprised of one or more “locked nucleic acids”. A locked nucleic acid (LNA), also termed “inaccessible RNA”, is a modified RNA nucleotide. The ribose moiety of an LNA nucleotide is modified with an extra bridge connecting the 2′ and 4′ carbons. The bridge “locks” the ribose in the 3′-endo structural conformation, which is often found in the A-form of DNA or RNA. LNA nucleotides can be mixed with DNA or RNA bases in the oligonucleotide whenever desired. The locked ribose conformation enhances base stacking and backbone pre-organization significantly increasing the thermal stability (melting temperature) of oligonucleotides. LNA bases may be comprised in a DNA backbone, but they can also be comprised in a backbone of LNA, 2′-O-methyl RNA, 2′-methoxyethyl RNA, or 2′-fluoro RNA. These molecules may comprise either a phosphodiester or phosphorothioate backbone.

The antisense oligonucleotides may comprise peptide nucleic acids (PNAs), which contain a peptide-based backbone rather than a sugar-phosphate backbone. Peptide nucleic acids (PNA) are synthetic analogs of DNA with a repeating N-(2-aminoethyl)-glycine peptide backbone connected to purine and pyrimidine nucleobases via a linker. In some embodiments, the sugar moiety of the nucleotide can be replaced, for example, with a non-sugar moiety such as a PNA.

Other chemical modifications that the antisense oligonucleotides may contain include, but are not limited to, sugar modifications, such as T-O-alkyl (e.g. 2′-O-methyl, 2′-O-methoxyethyl), 2′-fluoro, and 4′ thio modifications, and backbone modifications, such as one or more phosphorothioate, phosphorodiamidate Morpholino oligomers (PMOs) or phosphonocarboxylate linkages. It is known in the art that a methylene bridge between the 2′-oxygen and the 4′-carbon atoms provide higher structural rigidity and increased selective affinity to the RNA counter strand. It will also be appreciated that phosphorothioate backbone modifications adequately stabilize the oligonucleotides against degradation, and result in a high degree of binding to plasma proteins, which reduces rapid elimination. The antisense oligonucleotide can include a phosphorothioate in at least the first, second, or third internucleotide linkage at the 5′ or 3′ end of the nucleotide sequence. In an embodiment, the antisense oligonucleotide comprises 2 phosphorothioate linkages at the 5′ end and 4 phosphorothioate linkages at the 3′ end, and optionally a cholesterol modification at the 3′ end. Phosphorothioates are distributed to nearly all organs and tissues (a notable exception being the brain), but show a preference for liver and kidney. Additional 2′-methoxy or methoxyethylene modifications increase the stability and allow for lower doses (Baumann, V., & Winkler, J. (2014). miRNA-based therapies: strategies and delivery platforms for oligonucleotide and non-oligonucleotide agents. Future medicinal chemistry, 6(17), 1967-1984).

Other modifications of antisense oligonucleotides to enhance stability and improve efficacy, such as those described in Urban E, Structural modifications of antisense oligonucleotides Farmaco. 2003 March; 58(3):243-58., which is herein incorporated by reference in its entirety, are known in the art and are suitable for use in the methods of the invention.

In an embodiment, the antisense oligonucleotide is an antisense Morpholino. A Morpholino (MO), also known as a Morpholino oligomer and as a phosphorodiamidate Morpholino oligomer (PMO), is a type of oligomer molecule. Its molecular structure has subunits that are similar to DNA and RNA oligonucleotides, except that they have a backbone of methylenemorpholine rings linked through phosphorodiamidate groups. This feature still allows MOs to undergo Watson-Crick base pairing, but it offers significant advantages over conventional oligonucleotides. MOs do not act through an RNaseH mechanism but instead specifically binds to its selected target site to block access of cell components to that target site. This property can be exploited to block the translational start site of mRNA molecules, interfere with mRNA splicing, block miRNAs or their targets, and block ribozyme activity.

Translation Blocking: By sterically blocking the translation initiation complex, Morpholinos can knock down expression of many target sequences completely enough that after waiting for existing protein to degrade, the target protein band disappears from Western blots. Morpholinos generally do not cause degradation of their RNA targets; instead, they block the biological activity of the target RNA until that RNA is degraded naturally, which releases the Morpholino.

Splice Blocking: By blocking sites involved in splicing pre-mRNA, Morpholinos can be used to modify and control normal splicing events. This activity can be conveniently assayed by RT-PCR, with successful splice-modification appearing as changes in the RT-PCR product band on an electrophoretic gel. The band might shift to a new mass or, if splice-modification triggers nonsense-mediated decay of the transcript, the wild-spliced band will lose intensity or disappear.

In some embodiments, the antisense oligonucleotide is an antagomir (amiR). “Antagomirs” are single-stranded, chemically-modified ribonucleotides that are at least partially complementary to the miRNA sequence. The major difference with canonical antisense oligonucleotides is that antagomirs are designed to specifically target the “seed” sequence of a mature miRNA, thus blocking the processing of the miRNA on Ago2 and therefore inhibiting the function of the miRNA. Antagomirs may comprise one or more modified nucleotides, such as 2′-O-methyl-sugar modifications. In some embodiments, antagomirs comprise only modified nucleotides. Antagomirs may also comprise one or more phosphorothioate linkages resulting in a partial or full phosphorothioate backbone. The antagomir may be linked to a cholesterol or other moiety at its 3′ end to facilitate in vivo delivery and stability. Cholesterol-modified antagomir oligonucleotides have been shown to accumulate in the liver (Park J. K., miR-221 silencing blocks hepatocellular carcinoma and promotes survival, 2011, Cancer Res., 71(24): 7608-7616). An antagomir silences miRNA in a way still not completely known; it is thought that the miRNA/antagomir duplex induces degradation of the miRNA and recycling of the antagomir.

In an embodiment, the antisense oligonucleotides is a “gapmer”. Gapmers utilize the intracellular enzyme RNase H, which degrades the RNA strand in an RNA-DNA hetero-duplex. To prevent rapid catalysis, such antisense oligonucleotides are generally synthesized with a phosphorothioate backbone. To increase affinity and protect the oligonucleotides from exonucleases, a number of chemically modified nucleic acid analogs have been inserted at each end of the oligonucleotides to create what is called a gapmer. A gap with six to eight unmodified DNA nucleotides in the middle is mediating efficient induction of RNase H degradation. Very few base modifications are allowed within this DNA gap, in order not to disturb the catalysis. In some embodiments, suitable antisense molecule is a “gapmer”. In an embodiment, the gapmer is a 2′-O-methoxyethyl gapmer which contain 2′-O-methoxyethyl-modified ribonucleotides on both 5′ and 3′ ends with at least ten deoxyribonucleotides in the centre.

Preferably, the antisense oligonucleotide comprises a nucleotide sequence which is complementary to at least part of a nucleotide sequence present in SEQ ID NO: 1, SEQ ID NO:2, SEQ ID NO: 3 and/or SEQ ID NO:4.

TABLE 1
Sequences of miR-144.

| | | | | MiRBase |
|---|---|---|---|---|
| | | SEQ ID | | Accession |
| | Sequence (5′-3′) | NO | Species | Number |
| miR-144 | GGCUGGGAUAUCAUCAUAUA | 1 | Human | |
| (stem-loop) | CUGUAAGUUUGUGAUGAGA | | | |
| | CACUACAGUAUAGAUGAUGU | | | |
| | ACUAGUC | | | |
| miR-144-5p | GGAUAUCAUCAUAUACUGUA | 2 | Human | MIMAT0004600 |
| (mature) | AGU | | | |
| miR-144-3p | UACAGUAUAGAUGAUGUACU | 3 | Human | MIMAT0000436 |
| (mature) | | | | |
| miR-144 | GAUAUCA | 4 | Human | |
| (seed) | | | | |
| mmu-miR-144 | GGCUGGGAUAUCAUCAUAUA | 5 | Mouse | MI0000168 |
| (stem-loop) | CUGUAAGUUUGUGAUGAGA | | | |
| | CACUACAGUAUAGAUGAUGU | | | |
| | ACUAGUC | | | |
| mmu-miR-144- | GGAUAUCAUCAUAUACUGUA | 6 | Mouse | MIMAT0016988 |
| 5p | AGU | | | |
| mmu-miR-144- | UACAGUAUAGAUGAUGUACU | 7 | Mouse | MIMAT0000156 |
| 3p | | | | |

By “mature miRNA” we include the strand of a fully processed miRNA, or an siRNA that enters RISC. In some cases, miRNAs have a single mature strand that can vary in length between about 17-28 nucleotides in length. In other instances, miRNAs can have two mature strands, and again, the length of the strands can vary between about 17 and 28 nucleotides.

Antisense oligonucleotides may comprise a nucleotide sequence that is partially or substantially complementary to a precursor miRNA sequence (pre-miRNA) of miR-144. Antisense oligonucleotides may comprise a nucleotide sequence that is partially or substantially complementary to a stem loop miRNA sequence of miR-144. In one embodiment, an inhibitor of miR-144 is an antisense oligonucleotide comprising a sequence that is partially complementary to 5′-GGCUGGGAUAUCAUCAUAUACUGUAAGUUUGUGAUGAGACACUACAGUAUAGAU GAUGUACUAGUC-3′ (SEQ ID NO:1). In one embodiment, an inhibitor of miR-144 function is an antisense oligonucleotide having a sequence that is substantially complementary to a pre-miR-144 sequence of miR-144 (SEQ ID NO: 1). In some embodiments, the antisense oligonucleotide comprises a sequence that is substantially complementary to a sequence located outside the stem-loop region of the pre-miR-144 sequence.

In certain embodiments, an antisense oligonucleotide and a target nucleic acid are complementary to one another. In certain such embodiments, an antisense compound is perfectly complementary to a target nucleic acid. In certain embodiments, an antisense compound includes one mismatch. In certain embodiments, an antisense compound includes two mismatches. In certain embodiments, an antisense compound includes three or more mismatches.

As used herein, “complementary” and “complementarity” are interchangeable and refer to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G). Perfect complementarity or 100% complementarity refers to the situation in which each nucleotide of one polynucleotide strand or region can hydrogen bond with each nucleotide of a second polynucleotide strand or region. Less than perfect complementarity refers to the situation in which some, but not all, nucleotides of two strands or two regions can hydrogen bond with each other.

“Partially complementary” refers to a sequence that is at least about 50%, 55%, 60%, 65%, 70%, 75%, 80%, 85%, or 90% complementary to a target miRNA sequence.

“Substantially complementary” refers to a sequence that is at least about 90%, 91%, 92%, 93%, 94%, 95%, 96%, 97%, 98%, or 99% identical and complementary to a target miRNA sequence. We also include a sequence with 100% complementarity to the target miRNA sequence. We include that if two sequences are substantially complementary a duplex can be formed between them. The duplex may have one or more mismatches but the region of duplex formation is sufficient to down-regulate expression of the miRNA.

Methods for determining an appropriate number of mismatches between oligonucleotides, or between an oligonucleotide and a target nucleic acid, such as by determining melting temperature (Tm). Tm or Tn, can be calculated by techniques known in the art, such as those described in Freier et al. (Nucleic Acids Research, 1997, 25, 22: 4429-4443).

Preferably, the antisense oligonucleotide comprises a nucleotide sequence which is complementary to at least part of a nucleotide sequence present in a mature miR-144 sequence.

In an embodiment, the antisense oligonucleotide targets a mature sequence of miR-144 (e.g. SEQ ID NO: 2 and/or 3).

Preferably, the antisense oligonucleotide comprises a nucleotide sequence which is at least 50% complementary to SEQ ID NO: 1, SEQ ID NO:2 and/or SEQ ID NO:3.

Antisense oligonucleotides may comprise a sequence that is at least partially complementary to a mature miR-144 sequence, for example, at least about 50%, 55%, 60%, 65%, 70%, 75%, 80%, 85%, or 90% complementary to a mature miR-144 sequence.

In some embodiments, the antisense oligonucleotide may be substantially complementary to a mature miR-144 sequence, that is at least about 90%, 91%, 92%, 93%, 94%, 95%, 96%, 97%, 98%, or 99% complementary to a mature miR-144 sequence. In one embodiment, the antisense oligonucleotide comprises a sequence that is 100% complementary to a mature miR-144 sequence. In one embodiment, an inhibitor of miR-144 is an antisense oligonucleotide comprising a sequence that is partially complementary to 5′-GGAUAUCAUCAUAUACUGUAAGU-3′ (SEQ ID NO: 2) and/or 5′-UACAGUAUAGAUGAUGUACU-3′ (SEQ ID NO: 3). In one embodiment, an inhibitor of miR-144 is an antisense oligonucleotide comprising a sequence that is substantially complementary to 5′-GGAUAUCAUCAUAUACUGUAAGU-3′ (SEQ ID NO:2) and/or 5′-UACAGUAUAGAUGAUGUACU-3′ (SEQ ID NO: 3).

In certain such embodiments, the antisense oligonucleotide targeted to SEQ ID NO: 1 is at least 90% complementary to SEQ ID NO: 1. In certain such embodiments, the antisense oligonucleotide targeted to SEQ ID NO: 1 is at least 95% complementary to SEQ ID NO: 1. In certain such embodiments, the antisense oligonucleotide targeted to SEQ ID NO: 1 is 100% complementary to SEQ ID NO: 1. In certain such embodiments, the antisense oligonucleotide targeted to SEQ ID NO: 2 is at least 90% complementary to SEQ ID NO: 2. In certain such embodiments, the antisense oligonucleotide targeted to SEQ ID NO: 2 is at least 95% complementary to SEQ ID NO: 2. In certain such embodiments, the antisense oligonucleotide targeted to SEQ ID NO: 2 is 100% complementary to SEQ ID NO: 2. In certain such embodiments, the antisense oligonucleotide targeted to SEQ ID NO: 3 is at least 90% complementary to SEQ ID NO: 3. In certain such embodiments, the antisense oligonucleotide targeted to SEQ ID NO: 3 is at least 95% complementary to SEQ ID NO: 3. In certain such embodiments, the antisense oligonucleotide targeted to SEQ ID NO: 3 is 100% complementary to SEQ ID NO: 3. In certain such embodiments, the antisense oligonucleotide targeted to SEQ ID NO: 4 is at least 90% complementary to SEQ ID NO: 4. In certain such embodiments, the antisense oligonucleotide targeted to SEQ ID NO: 4 is at least 95% complementary to SEQ ID NO: 4. In certain such embodiments, the antisense oligonucleotide targeted to SEQ ID NO: 4 is 100% complementary to SEQ ID NO: 4.

By “mature miR-144 sequence” we include the strand of a fully processed miRNA, or siRNA that enters RISC. In some cases, miRNAs have a single mature strand that can vary in length between about 17-28 nucleotides in length. Alternatively, miRNAs can have two mature strands, and again, the length of the strands can vary between about 17 and 28 nucleotides.

Preferably, the nucleotide sequence which is complementary to at least part of a nucleotide sequence present in a miR-144 sequence is 15, 16, 17, 18, 19, 20, 20, 21, 22, or 23 nucleotides in length.

In some embodiments, the antisense oligonucleotide is for example an antagomir, and is from about 6 to about 30 nucleotides in length, from about 10 to about 30 nucleotides in length, from about 12 to about 28 nucleotides in length. Antisense oligonucleotide suitable for inhibiting miRNAs may be about 15 to about 50 nucleotides in length, more preferably about 18 to about 30 nucleotides in length, more preferably about 19 to about 30 nucleotides in length and most preferably about 19 to about 25 nucleotides in length. In some embodiments, the antisense oligonucleotide has a length of at least about 3, 4, 5, 6, 7, 8, 9, 10, 11, 12, 13, 14, 15, 16, 17, 18, 19, 20, 21, 22, 23, 24, 25, 26, 27, 28, 29, 30 nucleotides or more. In some embodiments, the antisense oligonucleotide has a length of at least 19 nucleotides.

In certain embodiments, the antisense oligonucleotide is 8 nucleotides in length. In certain embodiments, the antisense oligonucleotide is 9 nucleotides in length. In certain embodiments, the antisense oligonucleotide is 10 nucleotides in length. In certain embodiments, the antisense oligonucleotide is 11 nucleotides in length. In certain embodiments, the antisense oligonucleotide is 12 nucleotides in length. In certain embodiments, the antisense oligonucleotide is 13 nucleotides in length. In certain embodiments, the antisense oligonucleotide is 14 nucleotides in length. In certain embodiments, the antisense oligonucleotide is 15 nucleotides in length. In certain embodiments, the antisense oligonucleotide is 16 nucleotides in length. In certain embodiments, the antisense oligonucleotide is 17 nucleotides in length. In certain embodiments, the antisense oligonucleotide is 18 nucleotides in length. In certain embodiments, the antisense oligonucleotide is 19 nucleotides in length. In certain embodiments, the antisense oligonucleotide is 20 nucleotides in length.

In some embodiments, the antisense oligonucleotide is an antagomir, and is from about 6 to about 30 nucleotides in length, from about 10 to about 30 nucleotides in length, from about 12 to about 28 nucleotides in length, from about 19 to about 25 nucleotides in length. Antagomirs suitable for inhibiting miRNAs may be about 15 to about 50 nucleotides in length, more preferably about 18 to about 30 nucleotides in length, and most preferably about 19 to about 25 nucleotides in length. In some embodiments, the antagomir of a miRNA molecule has a length of at least about 3, 4, 5, 6, 7, 8, 9, 10, 11, 12, 13, 14, 15, 16, 17, 18, 19, 20, 21, 22, 23, 24, 25, 26, 27, 28, 29, 30 nucleotides or more. In some embodiments, the antagomir of a miRNA molecule has a length of at least 19 nucleotides.

In an embodiment, the antisense oligonucleotide is substantially single-stranded and comprises a sequence that is substantially complementary to 19 contiguous nucleotides of a nucleotide sequence of the mature miR-144 or pre-miR-144 nucleotide sequence and/or substantially complementary to 6 contiguous nucleotides in the seed sequence of a nucleotide sequence of the mature miR-144 or pre-miR-144 nucleotide sequence. Preferably, the antisense oligonucleotide comprises a nucleotide sequence which differs by no more than 1, 2, 3, 4 or 5 nucleotides from a pre-miR-144 nucleotide sequence.

Preferably, the antisense oligonucleotide comprises a nucleotide sequence which differs by no more than 1, 2, 3, 4 or 5 nucleotides from a mature miR-144 nucleotide sequence.

Preferably, the antisense oligonucleotide comprises a nucleotide sequence which is at least 80%, 85%, 90%, 95% or 100% complementary to SEQ ID NO: 4.

In an embodiment, the antisense oligonucleotide targets a seed sequence of miR-144 (e.g. SEQ ID NO: 4). The seed region is a 6-8 nucleotide-long sequence at the 5′ end of miRNA (nucleotide positions 2-7 or 2-8 of the mature miRNA).

Antisense oligonucleotides may comprise a sequence that is at least partially complementary to miR-144 sequence, for example, at least about 50%, 55%, 60%, 65%, 70%, 75%, 80%, 85%, or 90% complementary to the seed sequence of miR-144 sequence. In some embodiments, the antisense oligonucleotide may be substantially complementary to the seed sequence of miR-144 sequence, that is at least about 90%, 91%, 92%, 93%, 94%, 95%, 96%, 97%, 98%, or 99% complementary to a mature miR-144 sequence. In one embodiment, the antisense oligonucleotide comprises a sequence that is 100% complementary to the seed sequence of miR-144 sequence. In one embodiment, an inhibitor of miR-144 is an antisense oligonucleotide comprising a sequence that is partially complementary to 5′-GAUAUCA-3′ (SEQ ID NO:4). In one embodiment, an inhibitor of miR-144 is an antisense oligonucleotide comprising a sequence that is substantially complementary to 5′-GAUAUCA-3′ (SEQ ID NO:4).

In some embodiments, inhibitors of miR-144 are antagomirs comprising a sequence that is complementary to a mature miR-144 sequence. In one embodiment, an inhibitor of miR-144 is an antagomir having a sequence that is partially or substantially complementary to (SEQ ID NO: 1). In another embodiment, an inhibitor of miR-144 is an antagomir having a sequence that is partially or substantially complementary to (SEQ ID NO: 2). In another embodiment, an inhibitor of miR-144 is an antagomir having a sequence that is partially or substantially complementary to (SEQ ID NO: 3). In another embodiment, an inhibitor of miR-144 is an antagomir having a sequence that is partially or substantially complementary to (SEQ ID NO: 4).

As discussed above, in some embodiments, inhibitors of miR-144 are chemically-modified antisense oligonucleotides. In one embodiment, an inhibitor of miR-144 is a chemically-modified antisense oligonucleotide comprising a sequence substantially complementary to (SEQ ID NO: 1). In another embodiment, an inhibitor of miR-144 is a chemically-modified antisense oligonucleotide comprising a sequence substantially complementary to (SEQ ID NO: 2). In another embodiment, an inhibitor of miR-144 is a chemically-modified antisense oligonucleotide comprising a sequence substantially complementary to (SEQ ID NO: 3). In another embodiment, an inhibitor of miR-144 is a chemically-modified antisense oligonucleotide comprising a sequence substantially complementary to (SEQ ID NO: 4).

In a preferred embodiment, the agent is one that is an antisense oligonucleotide targeted to miR-144, for example miRIDIAN microRNA mmu-miR-144-5p hairpin inhibitor commercially available from Dharmacon; IH-301058-02, or miRIDIAN microRNA hsa-miR-144-3p hairpin inhibitor commercially available from Dharmacon; IH-300612-06.

In certain embodiments, an antisense oligonucleotide targeted to miR-144 comprises a nucleotide sequence selected from the nucleotide sequences set forth in Table 2. In certain embodiments, an antisense oligonucleotide targeted to any of SEQ ID NOs: 1-2 and 4 comprises a nucleotide sequence selected from the nucleotide sequences set forth in Table 2.

The nucleotide sequences set forth in SEQ ID NOs in Table 2 are independent of any modification. As such, antisense oligonucleotides defined by a SEQ ID NO may comprise, independently, one or more modifications as described herein.

Tables 2 illustrates examples of antisense oligonucleotides targeted to miR-144.

In an embodiment, the antisense oligonucleotide comprises a nucleotide sequence which is identical to at least part of a nucleotide sequence present in SEQ ID NO: 8, SEQ ID NO:9, SEQ ID NO: 10, SEQ ID NO:11, SEQ ID NO:12, and/or SEQ ID NO:13.

TABLE 2
Examples of antisense
oligonucleotides targeted to miR-144

| Target | Sequence (5′-3′) | SEQ ID NO |
|---|---|---|
| miR-144 | ACUACAGUAUAGAUGAUGUCU | 8 |
| miR-144 | ACUACAGUAUAGAUGAUAUCU | 9 |
| miR-144 | CUUACAGUAUAUGAUGAUAUC | 10 |
| miR-144 | AGUACAUCAUCUAUACUGUA | 11 |
| miR-144 | (X)ₙUGAUGUC(X)ₙ | 12 |
| miR-144 | (X)ₙUGAUAUC(X)ₙ | 13 |

In one embodiment, “X” is any nucleotide and “n” is an integer from 1-10. In an embodiment, n is any of 1, 2, 3, 4, 5, 6, 7, 8, 9 or 10.

In an embodiment, the sequences in Table 2 can comprise one or more chemical modification as discussed herein. In an embodiment, SEQ ID NO: 10 is modified as follows: 5′-mC/ZEN/mU mUmAmC mAmGmU mAmUmA mUmGmA mUmGmA mUmAmU mC/3ZEN/-3′, wherein “m” represents a 2′-O-methyl-modified oligonucleotide, and “ZEN” represents N,N-diethyl-4-(4-nitronaphthalen-1-ylazo)-phenylamine, for improved binding affinity and reduced exonuclease degradation.

In an embodiment, SEQ ID NO: 11 is modified as follows: 5′-A_{S}G_{S}UACAUCAUCUAUACU_{S}G_{S}U_{S}A_{S}-Chol-3′, wherein subscript ‘s’ represents a phosphorothioate linkage, and ‘Choi’ represents linked cholesterol. As discussed above, one or more of the nucleotides may be 2′-O-methyl-modified oligonucleotides.

Antisense oligonucleotides, or a part thereof, may have a defined percent identity to a SEQ ID NO disclosed in Table 2. As used herein, a sequence is identical to the sequence disclosed herein if it has the same nucleotide pairing ability. For example, an RNA which contains uracil in place of thymidine would be considered identical as they both pair with adenine. This identity may be over the entire length of the oligomeric compound, or in a part of the antisense oligonucleotide (e.g., nucleotides 1-20 of a 27-mer may be compared to a 20-mer to determine percent identity of the oligomeric compound to the SEQ ID NO. It is understood by those skilled in the art that an antisense oligonucleotide need not have an identical sequence to those described herein in order to function similarly to an antisense oligonucleotide described herein.

Percent identity is calculated according to the number of bases that have identical base pairing corresponding to the SEQ ID NO or antisense oligonucleotide to which it is being compared. The non-identical bases may be adjacent to each other, dispersed throughout the oligonucleotide, or both. For example, a 16-mer having the same sequence as nucleotides 2-17 of a 20-mer is 80% identical to the 20-mer. Alternatively, a 20-mer containing four nucleotides not identical to the 20-mer is also 80% identical to the 20-mer. A 14-mer having the same sequence as nucleotides 1-14 of an 18-mer is 78% identical to the 18-mer. Such calculations are known to the skilled person.

In a further example, a 30 nucleotide antisense oligonucleotide comprising the full sequence of the complement of a 20 nucleotide target sequence would comprise a portion of 100% identity with the complement of the 20 nucleotide target sequence, while further comprising an additional 10 nucleotide portion. In preferred embodiments, the oligonucleotides provided herein are at least 80%, at least 85%, at least 90%, at least 95%, at least 96%, at least 97%, at least 98%, at least 99% or 100% identical to at least a portion of the complement of the target sequence (i.e. miR-144) disclosed herein.

The percent sequence identity between two nucleic acid molecules may be determined using suitable computer programs, for example the Needle (EMBOSS) alignment tool (Madeira F et al. Nucleic Acids Res. 2019 Apr. 12).

In certain embodiments, antisense oligonucleotides may comprise a sequence that is at least about 50%, 55%, 60%, 65%, 70%, 75%, 80%, 85%, or 90% identical to any of the sequences set out in Table 2. In some embodiments, the antisense oligonucleotide may be at least about 90%, 91%, 92%, 93%, 94%, 95%, 96%, 97%, 98%, or 99% identical to any of the sequences set out in Table 2.

In other embodiments of the invention, inhibitors of miR-144 may be inhibitory RNA molecules, such as ribozymes, miRNA sponges, siRNAs, or shRNAs.

Another approach for inhibiting the function of miR-144 is administering an inhibitory RNA molecule having a double stranded region that is at least partially identical and partially complementary to the mature miR-144 sequence. The inhibitory RNA molecule may be a double-stranded, small interfering RNA (siRNA) or a short hairpin RNA molecule (shRNA) comprising a stem-loop structure.

The terms “small interfering RNA”, “short interfering RNA”, “small hairpin RNA”, “siRNA”, and shRNA are used interchangeably and refer to any nucleic acid molecule capable of mediating RNA interference (RNAi) or gene silencing. In one embodiment, the siRNA comprises a double stranded polynucleotide molecule comprising complementary sense and antisense regions, wherein the antisense region comprises a sequence complementary to a region of a target nucleic acid molecule (for example, a nucleic acid molecule encoding BRCAA1). In another embodiment, the siRNA comprises a single stranded polynucleotide having self-complementary sense and antisense regions, wherein the antisense region comprises a sequence complementary to a region of a target nucleic acid molecule. In another embodiment, the siRNA comprises a single stranded polynucleotide having one or more loop structures and a stem comprising self complementary sense and antisense regions, wherein the antisense region comprises a sequence complementary to a region of a target nucleic acid molecule, and wherein the polynucleotide can be processed either in vivo or in vitro to generate an active siRNA capable of mediating RNAi. As used herein, siRNA molecules need not be limited to those molecules containing only RNA, but further encompass chemically modified nucleotides and non-nucleotides.

Preferably, the inhibitory RNA molecule comprises a double-stranded region, and preferably wherein the double-stranded region comprises a nucleotide sequence that is substantially identical and substantially complementary to at least part of a nucleotide sequence present in SEQ ID NO: 1, SEQ ID NO:2, SEQ ID NO: 3 and/or SEQ ID NO: 4.

Preferably, the double-stranded region comprises a nucleotide sequence which is at least 50% complementary to at least part of a nucleotide sequence present in SEQ ID NO: 1, SEQ ID NO:2 and/or SEQ ID NO:3.

Preferably, the double-stranded region comprises a nucleotide sequence that is substantially identical and substantially complementary to a mature miR-144 sequence.

The double-stranded regions of the inhibitory RNA molecule may comprise a sequence that is at least partially identical and partially complementary, e.g. about 50%, 55%, 60%, 65%, 70%, 75%, 80%, 85%, or 90% identical and complementary, to the mature miRNA sequence. In some embodiments, the double-stranded regions of the inhibitory RNA comprise a sequence that is at least substantially identical and substantially complementary to the mature miRNA sequence.

By “partially identical and partially complementary” we include a sequence that is at least about 95%, 96%, 97%, 98%, or 99% identical and complementary to a target polynucleotide sequence.

By “substantially identical and substantially complementary” we include a sequence that is at least about 90%, 91%, 92%, 93%, 94%, 95%, 96%, 97%, 98%, or 99% identical and complementary to a target polynucleotide sequence. In other embodiments, the double-stranded regions of the inhibitory RNA molecule may contain 100% identity and complementarity to the target miRNA sequence. As discussed above, if two sequences are substantially complementary a duplex can be formed between them. The duplex may have one or more mismatches but the region of duplex formation is sufficient to down-regulate expression of the miRNA.

In one embodiment, an inhibitor of miR-144 is an inhibitory RNA molecule comprising a double-stranded region, wherein the double-stranded region comprises a sequence having 100% identity and complementarity to a mature miR-144 sequence (e.g. SEQ ID NO: 2 and/or SEQ ID NO:3). In another embodiment, an inhibitor of miR-144 is an inhibitory RNA molecule comprising a double-stranded region, wherein the double-stranded region comprises a sequence having 100% identity and complementarity to a seed miR-144 sequence (e.g. SEQ ID NO: 4). In some embodiments, inhibitors of miR-144 function are inhibitory RNA molecules which comprise a double-stranded region, wherein said double-stranded region comprises a sequence of at least about 50%, 55%, 60%, 65%, 705, 75%, 80%, 85%, 90%, 95%, 96%, 97%, 98%, or 99% identity and complementarity to a mature miR-144 sequence.

In an embodiment the inhibitory RNA molecule is a ribozyme. A ribozyme, or RNA enzyme, is an RNA molecule that has specific catalytic domains that possess endonuclease activity. A DNAzyme, or dezoxyribozyme, is a catalytic DNA that site specifically cleaving the target RNA.

Presently, at least six basic varieties of naturally-occurring enzymatic RNAs are known. Ribozymes act via Watson-Crick base pairing to a complementary target sequence, then site-specific cleavage of the substrate. After an enzymatic nucleic acid has bound and cleaved its RNA target, it is released from that RNA to search for another target and can repeatedly bind and cleave new targets.

Important characteristics of enzymatic nucleic acid molecules in the context of the present invention are that they have a specific substrate binding site which is complementary to at least part of miR-144, and that they have nucleotide sequences within or surrounding that substrate binding site which impart an RNA cleaving activity to the molecule.

Methods of producing a ribozyme targeted to any polynucleotide sequence are known in the art. Ribozymes may be designed as described in World of small RNAs: from ribozymes to siRNA and miRNA. Kawasaki H, Differentiation. 2004 March; 72(2-3):58-64. Ribozyme activity can be optimized by altering the length of the ribozyme binding arms or chemically synthesizing ribozymes with modifications that prevent their degradation by serum ribonucleases (see e.g., World of small RNAs: from ribozymes to siRNA and miRNA. Kawasaki H, Differentiation. 2004 March; 72(2-3):58-64.

In an embodiment the inhibitory RNA molecule is a miRNA sponge. Synthetic miRNA sponges are usually plasmid or viral vectors which contain tandomly arrayed miRNA binding sites of between 4-10 sites, separated with a small nucleotide spacer and inserted into a 3′UTR of the reporter gene driven by an RNA polymerase II promoter. Once inside the cells the sponges are amplified by the cell's native RNA polymerase II (see Ebert M S, Sharp P A. MicroRNA sponges: progress and possibilities. RNA. 2010; 16(11):2043-2050). When delivered into cells, the binding sites serve as decoys for the targeted miRNA (i.e. miR-144). Inclusion of an open reading frame for a selectable marker or reporter gene in the vector allows for selection or screening, fluorescence-activated cell sorting, or even laser capture microdissection of cells strongly expressing the sponge. It will be appreciated that regulatory elements could be included in the sponge promoter to make it drug-inducible or tissue-specific for the tissue of choice (i.e. the liver).

Preferably, the double-stranded region comprises a nucleotide sequence which is at least 80%, 85%, 90%, 95% or 100% complementary to SEQ ID NO: 4.

In an embodiment, the double-stranded region comprises a nucleotide sequence which is identical to at least part of a nucleotide sequence present in SEQ ID NO: 8, SEQ ID NO:9, SEQ ID NO: 10, SEQ ID NO:11, SEQ ID NO:12, and/or SEQ ID NO:13.

Inhibitory RNA molecules, or a part thereof, may have a defined percent identity to a SEQ ID NO disclosed in Table 2.

In certain embodiments, inhibitory RNA molecules may comprise a sequence that is at least about 50%, 55%, 60%, 65%, 70%, 75%, 80%, 85%, or 90% identical to any of the sequences set out in Table 2. In some embodiments, the antisense oligonucleotide may be at least about 90%, 91%, 92%, 93%, 94%, 95%, 96%, 97%, 98%, or 99% identical to any of the sequences set out in Table 2.

Suitable methods for delivery of the agent to the liver include any method by which a nucleic acid (e.g., RNA, DNA, including viral and non-viral vectors) can be introduced into an organelle, a cell, a tissue or an organism, as described herein or as would be known to one of ordinary skill in the art. The following review provides several ways of formulating an RNA molecule in order to optimize its internalisation into a cell (Kim S S., et al, Trends Mol. Med., 2009, 15: 491-500).

Preferably, the agent is delivered to cells of the liver using any of:
a physical method, such as: parenteral administration, direct injection or electroporation;
a delivery vehicle such as: a glucan-containing particle, lipid containing vesicle, viral containing vehicles, polymer containing vehicles, peptide containing vehicles, and exosomes.

In an embodiment, the delivery vehicle includes more than one component. For example, it can include one or more lipid moiety, one or more peptide, one or more polymer, one or more viral vector, or a combination thereof.

For example, in certain embodiments of the invention, the inhibitor of miR-144 may be administered by physical methods, for example by parenteral administration, such as intravenous injection, or subcutaneous injection, or by direct injection into the tissue (e.g. into liver tissue). In some embodiments, the inhibitor of miR-144 may be administered by oral, transdermal, intraperitoneal, subcutaneous, sustained release, controlled release, delayed release, suppository, or sublingual routes of administration. In other embodiments, the modulator of miR-144 may be administered by a catheter system.

In a preferred embodiment, the inhibitor of miR-144 is delivered by intravenous administration.

Physical methods for delivery to the liver are known in the art and include intrahepatic delivery by needle injection, gene gun (ballistic bombardment), electroporation, ultrasound-mediated delivery (sonoporation), and hydrodynamic delivery (Kamimura K, Liu D. Physical approaches for nucleic acid delivery to liver. AAPS J. 2008; 10(4):589-595).

In certain embodiments of the invention, the inhibitor of miR-144 may be administered by a glucan-containing particle. As used herein, “glucan-encapsulated” can refer to a formulation that provides the nucleic acid agent with full encapsulation, partial encapsulation, or both. In some embodiments, the nucleic acid agent is fully encapsulated in the glucan formulation (e.g., to form a nucleic acid-glucan particle).

Methods for making glucan-containing particles are known in the art, and described in the Examples, see for example WO2014134509 (incorporated by reference) which discloses peptide-modified glucan particles (PcGPs) and/or amine-modifies glucan particles (amGPs) for use in delivering payload molecules, in particular, nucleic acid payload molecules, to cells. Methods of making such particles and methods of using such particles, for example, for in mediating in vitro and in vivo gene silencing are also disclosed therein. Methods and compositions for delivering agents (e.g., gene silencing agents such as nucleic acids) and molecules to cells using yeast cell wall particles are disclosed in WO2009058913, incorporated by reference.

In an embodiment, the nucleic acid agent is encapsulated in micrometer-sized glucan shells (glucan-encapsulated siRNA particles, GeRPs) extracted from Saccharomyces cerevisiae and composed mainly of β-1,3-d-glucan, a ligand of the dectin-1 receptor and other receptors that are expressed by macrophages.

In certain embodiments of the invention, the inhibitor of miR-144 may be administered by a lipid containing vesicle. By “lipid containing vesicle” or “lipid particle” we include any lipid composition that can be used to deliver an agent to a subject, including but not limited to, lipid nanoparticles and liposomes, wherein an aqueous volume is encapsulated by an amphipathic lipid bilayer; or wherein the lipids coat an interior comprising a large molecular component, such as a plasmid comprising an interfering RNA sequence, with a reduced aqueous interior; or lipid aggregates or micelles, wherein the encapsulated component is contained within a relatively disordered lipid mixture. Lipid particles are directed to the liver mainly because of their size.

By “liposome” we include a vesicle composed of amphiphilic lipids arranged in a spherical bilayer or bilayers.

Liposomes are unilamellar or multilamellar vesicles which have an aqueous interior and a membrane formed from a lipophilic material. The aqueous interior contains the active agent/drug. The liposomal membrane is structurally similar to biological membranes, and therefore when liposomes are applied to a tissue, the liposomes start to merge with the cellular membranes and as the merging of the liposome and cell progresses, the liposomal contents are emptied into the target cell where the active agent may act. Thus, liposomes are useful for the transfer and delivery of active ingredients to the target site.

Liposomes fall into two broad classes. Cationic liposomes possess the advantage of being able to fuse to the cell wall. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm. In an embodiment, the agent is complexed with a lipid such as a cationic lipid. The cationic lipid may be one or more of N,N-dioleyl-N,N-dimethylamnnonium chloride (DODAC), N,N-distearyl-N,N-dimethylammonium bromide (DDRB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), and N,N-dimethyl-2,3-dioleyloxy)propylamine (DODMA), and a mixture thereof.

Non-cationic liposomes, although not able to fuse as efficiently with the cell wall, are taken up by macrophages in vivo. Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA to cell monolayers in culture. In an embodiment, the agent is complexed with a lipid such as a non-cationic lipid. The non-cationic lipid may be one or more of dioleoylphosphatidylethanolamine (DOPE), palmitoyloleoylphosphatidylcholine (POPC), egg phosphatidylcholine (EPC), distearoylphosphatidylcholine (DSPC), cholesterol, and combinations thereof.

Various liposomes comprising one or more glycolipids are known in the art Kraft J C, Emerging Research and Clinical Development Trends of Liposome and Lipid Nanoparticle Drug Delivery Systems. Journal of pharmaceutical sciences. 2014; 103(1):29-52. Many liposomes comprising lipids derivatised with one or more hydrophilic polymers, and methods of preparation thereof, are known in the art. A liposome generally includes a plurality of components such as one or more of a cationic lipid (e.g. an amino lipid), a targeting moiety, a fusogenic lipid, and/or a PEGylated lipid.

By “fusogenic” we include the ability of a liposome, or other drug delivery system to fuse with membranes of a cell. The membranes can be either the plasma membrane or membranes surrounding organelles, such as endosomes.

Lipid containing vesicles and their method of preparation are disclosed in U.S. Pat. Nos. 5,705,385; 5,981,501; 5,976,567; 6,586,410; 6,534,484; WO 96/40964; and WO 00/62813. A number of liposomes comprising nucleic acids are known in the art, see for example WO 96/40062 which discloses a method for encapsulating high molecular weight nucleic acids in liposomes, which provides for high nucleic acid entrapment efficiencies. The resulting compositions provide enhanced in vitro and in vivo transfection. WO 97/04787 discloses compositions comprising an oligonucleotide 8 to 50 nucleotides in length, which is targeted to mRNA encoding human raf and is capable of inhibiting raf expression, entrapped in sterically stabilised liposomes.

The lipid containing vesicle may be a lipid nanoparticle (LNP). The use of LNPs for the delivery of nucleic acids is well known in the art (Zatsepin, Timofei S et al. “Lipid nanoparticles for targeted siRNA delivery—going from bench to bedside.” International journal of nanomedicine vol. 11 3077-86. 5 Jul. 2016). Exemplary nanoparticles are 300-200 nm in diameter with appropriate surface modifications, such as by PEG or vitamin E D-α-tocopheryl PEG succinate (TPGS). It will be appreciated that PEGylated phospholipids are used in many lipid-based drug carriers primarily because they increase stability and increase circulation lifetime.

Liposomes and LNPs are similar, but slightly different in composition and function. Both are lipid nanoformulations and drug delivery vehicles, transporting cargo of interest within a protective, outer layer of lipids. In application, however, LNPs can take a variety of forms. Traditional liposomes include one or more rings of lipid bilayer surrounding an aqueous pocket, but not all LNPs have a contiguous bilayer that would qualify them as lipid vesicles or liposomes. Some LNPs assume a micelle-like structure, encapsulating drug molecules in a non-aqueous core.

In the context of the present invention, lipid containing vesicles typically have a mean diameter of about 30 nm to about 150 nm, more typically about 50 nm to about 140 nm more typically about 60 nm to about 130 nm, more typically about 70 nm to about 110 nm, most typically about 70 nm to about 90 nm, and are substantially nontoxic.

In one embodiment, the lipid to drug ratio (mass/mass ratio) (e.g., lipid to nucleic acid ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1, or about 6:1, 7:1, 8:1, 9:1, 10:1, 11:1, 12:1, or 33:1.

In another embodiment, the lipid-containing vesicle include an affinity moiety or targeting ligand effective to bind specifically to target cells at which the therapy is aimed.

For example, in certain embodiments of the invention, the inhibitor of miR-144 may be administered by a polymer containing vehicles. Polymer containing vesicles are known in the art, see for example in Schmidt H., Therapeutic Oligonucleotides Targeting Liver Disease: TTR Amyloidosis, Molecules. 2015, 20(10): 17944-17975. In an embodiment, the agent is complexed with a polymer such as a cationic polymer to form a polymer containing vehicle. Exemplary cationic polymers include poly(L)lysine (PLL) and polyethylenimine (PEI).

In an embodiment, the delivery vehicle is a peptide containing vehicle, such as an endoporter. Endo-Porter is a weak-base amphiphilic peptide that delivers antisense oligomers and other non-ionic substances into the cytosol/nuclear compartment of cells by an endocytosis-mediated process that avoids damaging the plasma membrane of the cell.

For example, in certain embodiments of the invention, the inhibitor of miR-144 may be administered by exosomes. The use of exosomes for targeted drug delivery is known in the art, see for example Antimisiaris SGExosomes and Exosome-Inspired Vesicles for Targeted Drug Delivery. Pharmaceutics. 2018 Nov. 6; 10(4)

In an embodiment, the delivery vehicle is a viral containing vehicle, such as an expression vector. In an embodiment, an expression vector may be used to deliver an inhibitor of miR-144 to the liver. RNA molecules may be encoded by a nucleic acid molecule comprised in a vector. The term “vector” is used to refer to a carrier nucleic acid molecule into which a nucleic acid sequence can be inserted for introduction into a cell where it can be replicated. Examples of viral vectors include, but are not limited to, adenoviral vectors, adeno-associated virus vectors, retroviral vectors, and the like. An expression construct can be replicated in a living cell, or it can be made synthetically. As used herein, the terms “expression construct,” “expression vector,” and “vector,” are used interchangeably.

In one embodiment, an expression vector for expressing an inhibitor of miR-144 comprises a promoter operably linked to a polynucleotide encoding an antisense oligonucleotide, wherein the sequence of the expressed antisense oligonucleotide is partially or perfectly complementary to the mature miR-144 sequence. In an alternative embodiment, the expression vector for expressing an inhibitor of miR-144 comprises one or more promoters operably linked to a polynucleotide encoding a shRNA or siRNA, wherein the expressed shRNA or siRNA comprises a double stranded region that is partially or substantially identical and complementarity to the mature miR-144.

By “operably linked” or “under transcriptional control” we include that the promoter is in the correct location and orientation in relation to a polynucleotide to control the initiation of transcription by RNA polymerase and expression of the polynucleotide.

By “promoter” we include a DNA sequence recognized by the synthetic machinery of the cell, or introduced synthetic machinery, required to initiate the specific transcription of a gene. The use of viral, mammalian cellular, or bacterial phage promoters are well-known in the art to achieve expression of a coding sequence of interest and include the human cytomegalovirus (CMV) immediate early gene promoter, the SV40 early promoter, the Rous sarcoma virus long terminal repeat, rat insulin promoter and glyceraldehyde-3-phosphate dehydrogenase

There are a number of ways in which expression vectors may introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells. One of the preferred methods for in vivo delivery involves the use of an adenovirus expression vector.

By “adenovirus expression vector” we include those constructs containing adenovirus sequences sufficient to (a) support packaging of the construct and (b) to express a nucleic acid agent as described herein that has been cloned therein. The expression vector comprises a genetically engineered form of adenovirus. Adenovirus is known in the art to be suitable for use as a gene transfer vector because of its mid-sized genome, ease of manipulation, high titer, wide target cell range and high infectivity. Viral vectors may be injected directly into the afferent vessels of the liver (portal vein) or the bile duct instead of the peripheral circulation.

In another embodiment, liver-specific delivery can be achieved using synthetic compounds called synthetic vectors and targeted gene delivery through asialoglycoprotein receptor (ASGP-R), or the transferrin receptor.

More recently, targeted delivery of nucleic acids (siRNAs) to cells of the liver has been achieved by direct conjugation to a triantennary GalNAc sugar, for receptor-mediated endocytosis via the asialoglycoprotein receptor (ASGPR) that is primarily expressed on the surface of hepatocytes. The ASGPR is a C-type lectin receptor that facilitates the clearance of desialylated glycoproteins from the blood. It is expressed at high copy number (0.5-1 million per cell) on the surface of hepatocytes, and each hepatocyte may endocytose up to 5 million copies of ASGPR per hour, providing significant excess receptor availability for drug binding and uptake (Janas, Maja M et al. “The Nonclinical Safety Profile of GalNAc-conjugated RNAi Therapeutics in Subacute Studies.” Toxicologic pathology vol. 46, 7 (2018): 735-745).

In an embodiment, nucleic acid agents of the invention are administered in a therapeutically effective amount in a pharmaceutically acceptable carrier in doses ranging from (about) 0.01 ug to (about) 1 gm; such as (about) 1 mg to (about) 100 mg per kg of body weight depending on the age of the subject and the severity of the disorder or disease state being treated. In an embodiment, an antisense oligonucleotide is administered in doses ranging from 2-5 mg/Kg of body weight. In an embodiment, an antisense oligonucleotide is administered in doses ranging from 3-4 mg/Kg of body weight.

By “pharmacologically effective amount,” “therapeutically effective amount” or simply “effective amount” we include that amount of an agent, such as a nucleic acid agent, effective to produce the intended pharmacological, therapeutic or preventive result. without undesirable side effects (such as toxicity, irritation or allergic response). For example, if a given clinical treatment is considered effective when there is at least a 25% reduction in a measurable parameter (for example, miR-144 expression) associated with a disease or disorder, a therapeutically effective amount of a drug for the treatment of that disease or disorder is the amount necessary to effect at least a 25% reduction in that parameter. Although individual needs may vary, optimal ranges for effective amounts of the agent can be readily determined by the skilled person. It will be appreciated that human doses can be extrapolated from animal studies. Generally, the dosage required to provide an effective amount of the agent, which can be adjusted by one skilled in the art, will vary depending on the age, health, physical condition, weight, type and extent of the disease or disorder of the recipient, frequency of treatment, the nature of concurrent therapy (if any) and the nature and scope of the desired effect (5).

Following treatment, the subject is monitored for changes in his/her condition and for alleviation of the symptoms of the liver disease and/or liver condition. The dosage of the drug may either be increased if the subject does not respond significantly to current dosage levels, or the dose may be decreased if an alleviation of the symptoms of the disorder or disease state is observed, or if the disorder or disease state has been abated.

In an embodiment, one or more symptoms of the liver disease and/or liver condition is improved in the subject following administration of the agent, for example hepatocyte death, immune cell infiltration and/or fibrosis.

During hepatocyte death the cellular content of the soluble fraction of cytokeratin-18 (CK18), the major intermediate filament protein in the liver, is released into the extracellular space during cell death. Therefore, blood measurements of soluble full length, and/or CK18 fragments are indicative of hepatocyte cell death. These measurements can be carried out using techniques known in the art such as ELISA.

Inflammatory infiltrates in the liver can be measured by techniques known in the art such as immunohistochemistry on liver biopsies measuring infiltrating T cells and/or macrophages.

In an embodiment, the agent is administered in combination with an additional therapy.

Agents of the invention can be used in combination with the administration of conventional therapy used to treat the liver disease and/or condition.

In an embodiment, the additional therapy is a lipid-lowering therapy, such as HMG-CoA Reductase inhibitors.

By “lipid-lowering” we include a reduction in one or more serum lipids in a subject over time.

As used herein, the term “lipid-lowering therapy” refers to a therapeutic regimen provided to a subject to reduce one or more lipids in a subject. In certain embodiments, a lipid-lowering therapy is provided to reduce one or more of total cholesterol, ApoB, LDL-C, VLDL-C, IDL-C, non-HDL-C, triglycerides, small dense LDL particles, and Lp(a) in an individual.

HMG-CoA reductase inhibitors, also termed “statins” are a class of drugs that lower cholesterol levels in subjects with, or at risk of, cardiovascular disease. They lower cholesterol by inhibiting the enzyme HMG-CoA reductase, which is the rate-limiting enzyme of the mevalonate pathway of cholesterol synthesis. Inhibition of this enzyme in the liver results in decreased cholesterol synthesis as well as increased synthesis of LDL receptors, resulting in an increased clearance of low-density lipoprotein (LDL) from the bloodstream. Examples of statins may be selected from the group consisting of: Atorvastatin, Cerivastatin, Fluvastatin, Lovastatin, Mevastatin, Pitavastatin, Pravastatin, Rosuvastatin, and Simvastatin. Specific guidelines for statins treatment could be found in Table A (as described in American Heart Association guidelines):

| High Intensity | Moderate Intensity | Low Intensity |
|---|---|---|
| Lowers LDL-C | Lowers LDL-C | Lowers LDL-C |
| by ≥50% | by 30%-49% | by <30% |
| Atorvastatin | Atorvastatin | Simvastatn |
| (40 mg) 80 mg | 10 mg (20 mg) | 10 mg |
| Rosuvastatin | Rosuvastatin | Pravastatin |
| 20 mg (40 mg) | (5 mg) 10 mg | 10-20 mg |
| | Simvastatin | Lovastatin |
| | 20-40 mg | 20 mg |
| | Pravastatin | Fluvastatin |
| | 40 mg (80 mg) | 20-40 mg |
| | Lovastatin | |
| | 40 mg (80 mg) | |
| | Fluvastatin XL | |
| | 80 mg | |
| | Fluvastatin | |
| | 40 mg BID | |
| | Pitavastatin | |
| | 1-4 mg | |

Abbreviation: LDL-C, low-density lipoprotein cholesterol.

Boldface type indicates specific statins and doses that were evaluated in randomized controlled trials and the Cholesterol Treatment Trialists' 2010 meta-analysis. All these randomized controlled trials demonstrated a reduction in major cardiovascular events. Nonbold type indicates statins and doses that have been approved by the FDA but were not tested in the RCTs reviewed.

Preferably, administration of the agent delays and/or prevents the progression from NASH to fibrosis, cirrhosis and/or hepatocellular carcinoma in the subject.

Progression of NAFLD to HCC can be as follows: NAFLD/fatty liver to NASH, to NASH with mild fibrosis, to NASH with severe fibrosis, to cirrhosis, and finally to HCC.

Preferably, the agent is formulated and/or adapted for delivery and/or uptake by cells of the liver.

By “formulated and/or adapted for uptake by cells of the liver” we include that the agent is in a form, such as comprised within a particular vehicle, which results in its uptake by cells of the liver to a greater extent that the agent is taken up by cells of another organ type, such as the brain.

By “formulated and/or adapted for delivery to cells of the liver” we include that the agent is in a form, such as comprised within a particular vehicle, which results in its delivery to cells of the liver to a greater extent that the agent is delivered to cells of another organ type, such as the brain.

In an embodiment, the uptake of the agent by cells of the liver is receptor-mediated. Examples of receptor-mediated uptake into cells of the liver includes receptor-mediated endocytosis via the asialoglycoprotein receptor (ASGPR) that is primarily expressed on the surface of hepatocytes and receptor-mediated phagocytosis by macrophages and dendritic cells which express the dectin-1 receptor.

As discussed above, encapsulating the agent in micrometer-sized glucan extracted from Saccharomyces cerevisiae and composed mainly of β-1,3-d-glucan, which is a ligand of the dectin-1 receptor and other receptors that are expressed by macrophages, can aid receptor-mediated phagocytosis by macrophages of the liver.

In an embodiment, the agent is glucan encapsulated.

In a further aspect, the invention provides an agent that inhibits microRNA-144 (miR-144) for use in inhibiting progression of a liver disease and/or liver condition in which oxidative stress is a contributory factor in a subject.

In a further aspect, the invention provides use of an agent that inhibits microRNA-144 (miR-144) for the manufacture of a medicament for inhibiting progression of a liver disease and/or liver condition in which oxidative stress is a contributory factor in a subject.

In a further aspect, the invention provides a method for inhibiting progression of a liver disease and/or liver condition in which oxidative stress is a contributory factor in a subject, wherein the method comprises administering an agent that inhibits microRNA-144 (miR-144) to the subject.

Additionally, oxidative stress in the liver has been implicated in the progression of fatty liver (NAFLD) to NASH, fibrosis and hepatocellular carcinoma. The main mechanism protecting against oxidative stress is the Nuclear Factor Erythroid 2-Related Factor 2 (NRF2)/ARE pathway, which induces the expression of antioxidant response genes. This inappropriate lipid accumulation leads to oxidative stress and excessive production of reactive oxygen species (ROS).

By “inhibiting progression” we include progression of fatty liver (NAFLD) to NASH, to NASH with mild fibrosis, toNASH with severe fibrosis, to cirrhosis, to HCC.

In a further aspect, the invention provides a method for identifying a subject who is at risk of developing a liver disease and/or liver condition in which oxidative stress is a contributory factor, comprising:

a) obtaining and/or providing a test sample from a subject;

b) determining the expression and/or activity of miR-144 in the test sample,
wherein the expression and/or activity of miR-144 relative to a control indicates whether the subject is at risk of developing a liver disease and/or liver condition in which oxidative stress is a contributory factor.

In a particularly preferred embodiment, determination that the expression and/or activity of miR-144 is increased relative to a control indicates that the subject is at risk of developing a liver disease and/or liver condition in which oxidative stress is a contributory factor.

In an embodiment, the expression and/or activity of miR-144 is increased at least 2-fold in the test sample compared to the control sample.

Preferably, the method for identifying a subject who is at risk of developing a liver disease and/or liver condition in which oxidative stress is a contributory factor further comprises administering an effective amount of a therapy to the subject with a liver disease and/or liver condition in which oxidative stress is a contributory factor, for example wherein the method comprises administering an agent that inhibits miR-144.

In some embodiments, the expression and/or activity of miR-144 is measured after administering an agent that inhibits miRNA-144 to the subject. In some embodiments, the expression and/or activity of miR-144 is measured once or twice.

In a further aspect, the invention provides a method for identifying a subject who has a liver disease and/or liver condition in which oxidative stress is a contributory factor, comprising:

a) obtaining and/or providing a test sample from a subject;

b) determining the expression and/or activity of miR-144 in the test sample,
wherein the expression and/or activity of miR-144 relative to a control sample indicates whether the subject has a liver disease and/or liver condition in which oxidative stress is a contributory factor.

In a particularly preferred embodiment, determination that the expression and/or activity of miR-144 is increased relative to a control indicates that the subject has a liver disease and/or liver condition in which oxidative stress is a contributory factor.

In an embodiment, the expression and/or activity of miR-144 is increased at least 2-fold in the test sample compared to the control sample.

Preferably, the method for identifying a subject who has a liver disease and/or liver condition in which oxidative stress is a contributory factor further comprises administering an effective amount of a therapy to the subject with a liver disease and/or liver condition in which oxidative stress is a contributory factor, for example wherein the method comprises administering an agent that inhibits miR-144.

In some embodiments, the expression and/or activity of miR-144 is measured after administering an agent that inhibits miRNA-144 to the subject. In some embodiments, the expression and/or activity of miR-144 is measured once or twice.

In a further aspect, the invention provides a method for predicting the response of a subject having a liver disease and/or liver condition in which oxidative stress is a contributory factor, to treatment with an agent that inhibits microRNA-144 (miR-144), comprising:

a) obtaining and/or providing a test sample from a subject;

b) determining the expression and/or activity of miR-144 in the test sample,
wherein the expression and/or activity of miR-144 relative to a control sample indicates that the subject will respond to treatment with the agent.

In a particularly preferred embodiment, determination that the expression and/or activity of miR-144 is increased relative to a control indicates that the subject will respond to treatment with the agent.

In an embodiment, the expression and/or activity of miR-144 is increased at least 2-fold in the test sample compared to the control sample.

Preferably, the method for predicting the response of a subject having a liver disease and/or liver condition in which oxidative stress is a contributory factor further comprises administering an effective amount of a therapy to the subject with a liver disease and/or liver condition in which oxidative stress is a contributory factor, for example wherein the method comprises administering an agent that inhibits miR-144.

In some embodiments, the expression and/or activity of miR-144 is measured after administering an agent that inhibits miRNA-144 to the subject. In some embodiments, the expression and/or activity of miR-144 is measured once or twice.

Methods for determining the expression and/or activity of miR-144 are as described herein.

Examples of a test sample that can be used in the methods and uses of the invention include, but are not limited to a liver biopsy, blood plasma, and/or serum. By “serum”, we include the portion of plasma remaining after coagulation of blood.

A further aspect of the invention provides use of the expression and/or activity of miR-144 in identifying a liver disease and/or liver condition in which oxidative stress is a contributory factor in a subject, wherein the presence of an increased expression and/or activity of miR-144 in a test sample from the subject relative to a control sample, indicates that the subject has a liver disease and/or liver condition in which oxidative stress is a contributory factor.

In an embodiment, the use is an in vitro use.

Preferably, the use further comprises administering an effective amount of a therapy to the subject with a liver disease and/or liver condition in which oxidative stress is a contributory factor, for example administering an agent that inhibits miR-144.

Preferably, the liver disease and/or liver condition in which oxidative stress is a contributory factor is as defined in any preceding claim.

In an embodiment, the use comprises determining the expression and/or activity of miR-144 in a test sample from the subject and/or a control sample.

Preferably, the expression and/or activity of miR-144 is increased at least 2-fold in the test sample compared to the control sample.

Accordingly, the invention provides a method for diagnosing a liver disease and/or liver condition in which oxidative stress is a contributory factor in a subject comprising the steps of:
(a) providing a test sample from said subject;
(b) measuring the expression of miR-144;

wherein an elevated level of miR-144 in comparison to a control sample indicates the subject has, or is at risk of developing, a liver disease and/or liver condition in which oxidative stress is a contributory factor.

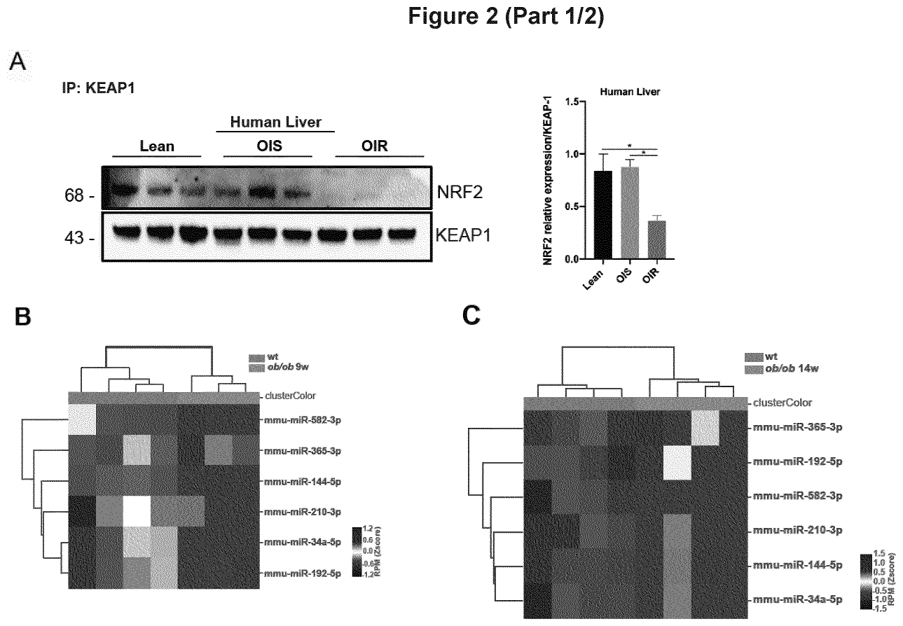

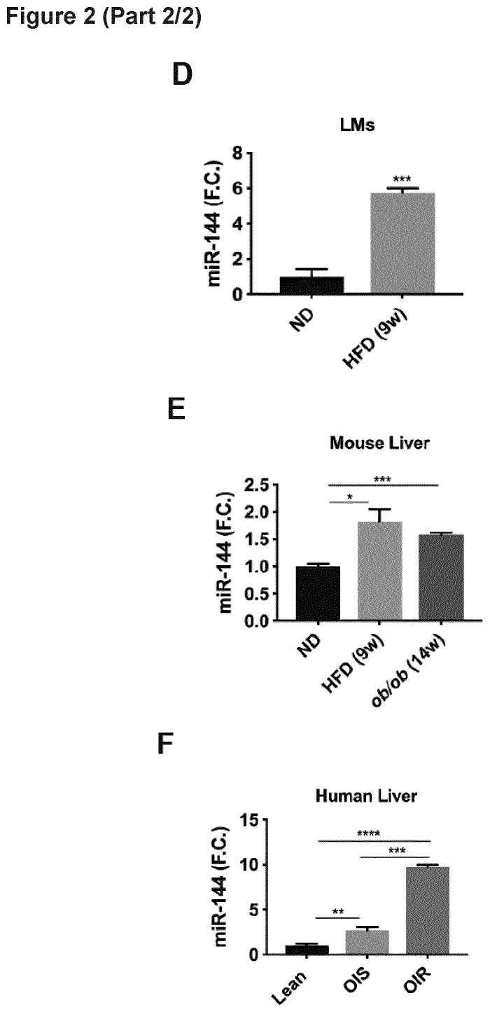

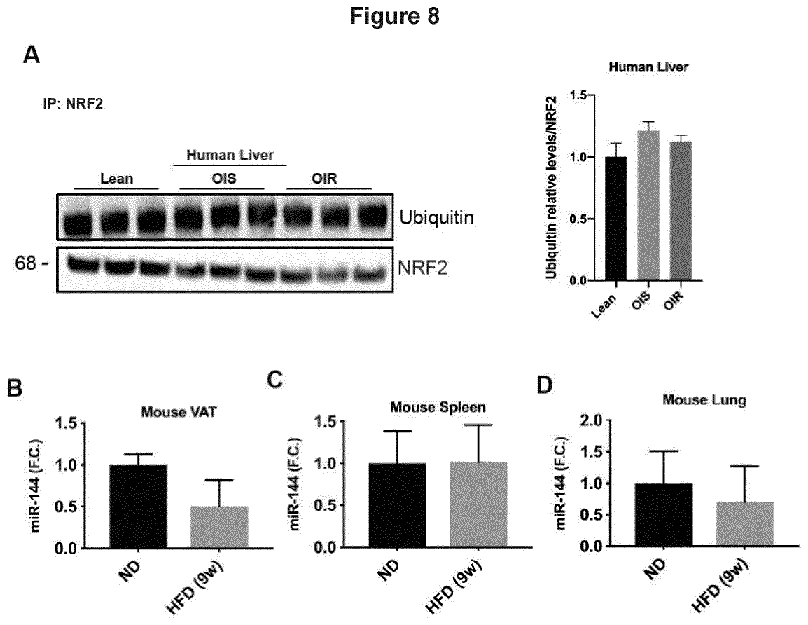

As can be seen from the accompanying Examples, the inventors observed increased miR-144 expression in isolated liver macrophages from high fat diet (HFD) mice (FIG. 2C), as well as in livers of both HFD-fed and ob/ob mice compared to their respective controls (FIG. 2D). The observed increase of miR-144 was liver specific, as its expression remained unchanged in the spleen, lung and visceral adipose tissue (VAT) isolated from obese mice (FIG. 8A-C(S2A-C)).

The expression level of the miR-144 may be determined either via microarray analyses, RT-PCR, Northern blotting, or other suitable methods described herein or known in the art.

In an embodiment, the test sample comprises one or more liver cells. In an alternative embodiment, the test sample does not comprise liver cells.

Examples of test sample that can be used in the methods and uses of the invention include, but are not limited to a liver biopsy, blood plasma, and/or serum. By “serum”, we include the portion of plasma remaining after coagulation of blood.

In an embodiment, the control sample comprises one or more liver cells in which there is no oxidative stress, for example liver cells from a subject without oxidative stress in the liver. In an alternative embodiment, the control sample does not comprise liver cells.

Examples of control samples that can be used in the methods and uses of the invention include, but are not limited to a liver, blood plasma, and/or serum sample from a lean and healthy subject.

The liver disease and/or liver condition in which oxidative stress is a contributory factor in a subject is as defined herein.

In another aspect, the invention provides a pharmaceutical composition comprising an agent which inhibits miR-144, which is formulated and/or adapted for delivery to phagocytic cells of the liver.

Preferably, the agent is as defined herein.

By “pharmaceutically acceptable” is included that the formulation is sterile and pyrogen free. Suitable pharmaceutical carriers, diluents and excipients are well known in the art of pharmacy. The carrier(s) must be “acceptable” in the sense of being compatible with the inhibitor and not deleterious to the recipients thereof. Typically, the carriers will be water or saline which will be sterile and pyrogen free; however, other acceptable carriers may be used.

In an embodiment, the pharmaceutical compositions or formulations of the invention are for parenteral administration, more particularly for intravenous administration. In a preferred embodiment, the pharmaceutical composition is suitable for intravenous administration to a patient, for example by injection.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

In an alternative preferred embodiment, the pharmaceutical composition is suitable for topical administration to a patient.

Preferably, the formulation is a unit dosage containing a daily dose or unit, daily sub-dose or an appropriate fraction thereof, of the active ingredient.

The agent or active ingredient may be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the agent or active ingredient will generally be administered in admixture with a suitable pharmaceutical excipient, diluent or carrier selected with regard to the intended route of administration and standard pharmaceutical practice.

For example, the agent or active ingredient may be administered orally, buccally or sublingually in the form of tablets, capsules, ovules, elixirs, solutions or suspensions, which may contain flavouring or colouring agents, for immediate-, delayed- or controlled-release applications. The active ingredient may also be administered via intracavernosal injection.

Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The agent or active ingredient can also be administered parenterally, for example, intravenously, intra-arterially, intraperitoneally, intrathecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of an agent, antibody or compound will usually be from 1 to 1,000 mg per adult (i.e. from about 0.015 to 15 mg/kg), administered in single or divided doses.

Thus, for example, the tablets or capsules of the agent or active ingredient may contain from 1 mg to 1,000 mg of agent or active agent for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The agent or active ingredient can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of an active ingredient and a suitable powder base such as lactose or starch. Such formulations may be particularly useful for treating solid tumours of the lung, such as, for example, small cell lung carcinoma, non-small cell lung carcinoma, pleuropulmonary blastoma or carcinoid tumour.

Aerosol or dry powder formulations are preferably arranged so that each metered dose or “puff” contains at least 1 mg of the inhibitor for delivery to the patient. It will be appreciated that the overall daily dose with an aerosol will vary from patient to patient, and may be administered in a single dose or, more usually, in divided doses throughout the day.

Alternatively, the agent or active ingredient can be administered in the form of a suppository or pessary, particularly for treating or targeting colon, rectal or prostate tumours.

The agent or active ingredient may also be administered by the ocular route. For ophthalmic use, the inhibitor can be formulated as, e.g., micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum. Such formulations may be particularly useful for treating solid tumours of the eye, such as retinoblastoma, medulloepithelioma, uveal melanoma, rhabdomyosarcoma, intraocular lymphoma, or orbital lymphoma.

The agent or active ingredient may be applied topically in the form of a lotion, solution, cream, ointment or dusting powder, or may be transdermally administered, for example, by the use of a skin patch. For application topically to the skin, the active ingredient can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water. Such formulations may be particularly useful for treating solid tumours of the skin, such as, for example, basal cell cancer, squamous cell cancer or melanoma.

Formulations suitable for topical administration in the mouth include lozenges comprising the active ingredient in a flavoured basis, usually sucrose and acacia or tragacanth; pastilles comprising the agent or active ingredient in an inert basis such as gelatin and glycerin, or sucrose and acacia; and mouth-washes comprising the active ingredient in a suitable liquid carrier. Such formulations may be particularly useful for treating solid tumours of the mouth and throat.

In an embodiment, the agent or active ingredient may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

The agent or active ingredient can be administered by a surgically implanted device that releases the drug directly to the required site, for example, into the eye to treat ocular tumours. Such direct application to the site of disease achieves effective therapy without significant systemic side-effects.

An alternative method for delivery of agents or active ingredients is the Regel injectable system that is thermo-sensitive. Below body temperature, Regel is an injectable liquid while at body temperature it immediately forms a gel reservoir that slowly erodes and dissolves into known, safe, biodegradable polymers. The active drug is delivered over time as the biopolymers dissolve.

Polypeptide pharmaceuticals can also be delivered orally. The process employs a natural process for oral uptake of vitamin B12 in the body to co-deliver proteins and peptides. By riding the vitamin B12 uptake system, the protein or peptide can move through the intestinal wall. Complexes are synthesised between vitamin B12 analogues and the drug that retain both significant affinity for intrinsic factor (IF) in the vitamin B12 portion of the complex and significant bioactivity of the drug portion of the complex.

Polynucleotides may be administered as a suitable genetic construct as described below and delivered to the patient where it is expressed. Typically, the polynucleotide in the genetic construct is operatively linked to a promoter which can express the compound in the cell. The genetic constructs of the invention can be prepared using methods well known in the art, for example in Sambrook et al (2001).

Although genetic constructs for delivery of polynucleotides can be DNA or RNA, it is preferred if they are DNA.

Preferably, the genetic construct is adapted for delivery to a human cell. Means and methods of introducing a genetic construct into a cell are known in the art, and include the use of immunoliposomes, liposomes, viral vectors (including vaccinia, modified vaccinia, lentivurus, parvovirus, retroviruses, adenovirus and adeno-associated viral (AAV) vectors), and by direct delivery of DNA, e.g. using a gene-gun and electroporation. Furthermore, methods of delivering polynucleotides to a target tissue of a patient for treatment are also well known in the art. In an alternative method, a high-efficiency nucleic acid delivery system that uses receptor-mediated endocytosis to carry DNA macromolecules into cells is employed. This is accomplished by conjugating the iron-transport protein transferrin to polycations that bind nucleic acids. High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used. It will be appreciated that “naked DNA” and DNA complexed with cationic and neutral lipids may also be useful in introducing the DNA of the invention into cells of the individual to be treated. Non-viral approaches to gene therapy are described in Ledley (1995, Human Gene Therapy 6, 1129-1144).

Although for cancer/tumours of specific tissues it may be useful to use tissue-specific promoters in the vectors encoding a polynucleotide inhibitor, this is not essential, as the risk of expression of the active ingredient in the body at locations other than the cancer/tumour would be expected to be tolerable in compared to the therapeutic benefit to a patient suffering from a cancer/tumour. It may be desirable to be able to temporally regulate expression of the polynucleotide inhibitor in the cell, although this is also not essential.

In an embodiment, the pharmaceutical composition comprises an agent that is encapsulated for receptor-mediated uptake by phagocytic cells of the liver.

In an embodiment, the pharmaceutical composition is formulated for injection.

A further aspect of the invention provides the pharmaceutical composition as described herein for use in medicine.

A further aspect of the invention provides a kit of parts, comprising the pharmaceutical composition as described herein and/or reagents for measuring the expression level of miR-144.

In some embodiments, the kits comprise one or more agent and/or compound herein. The kit can also contain instructions for use. In some embodiments, the kit could contain control samples (e.g. sample positive and negative for miR-144) and primers (specific for miR-144 and for miRNA as internal control, e.g. RNAU6) to measure the expression levels of miR-144 for diagnosis.

In a further aspect of the invention, kits used for administration of a compound herein to a subject are provided. In such instances, in addition to comprising at least one agent as described herein, the kit can further comprise one or more of the following: syringe, alcohol swab, cotton ball, and/or gauze pad. In some embodiments, an agent which inhibits miR-144 can be present in a pre-filled syringe rather than in a vial. A plurality of pre-filled syringes, such as 10, can be present in, for example, dispensing packs. The kit can also contain instructions for administering an agent described herein.

In an embodiment, the agent, use, method or composition substantially as shown and described herein, with reference to the accompanying description, examples and drawings.

All of the documents referred to herein are incorporated herein, in their entirety, by reference.

The listing or discussion of an apparently prior published document in this specification should not necessarily be taken as an acknowledgement that the document is part of the state of the art or is common general knowledge.

The invention will now be described by reference to the following Figures and Examples. Preferred, non-limiting examples which embody certain aspects of the invention will now be described, with reference to the following figures:

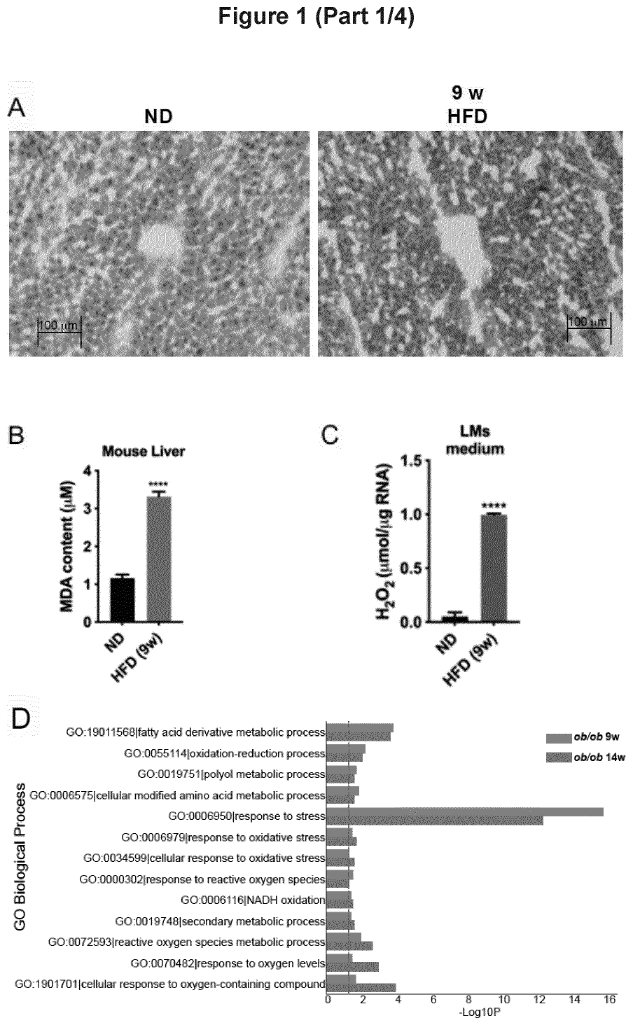

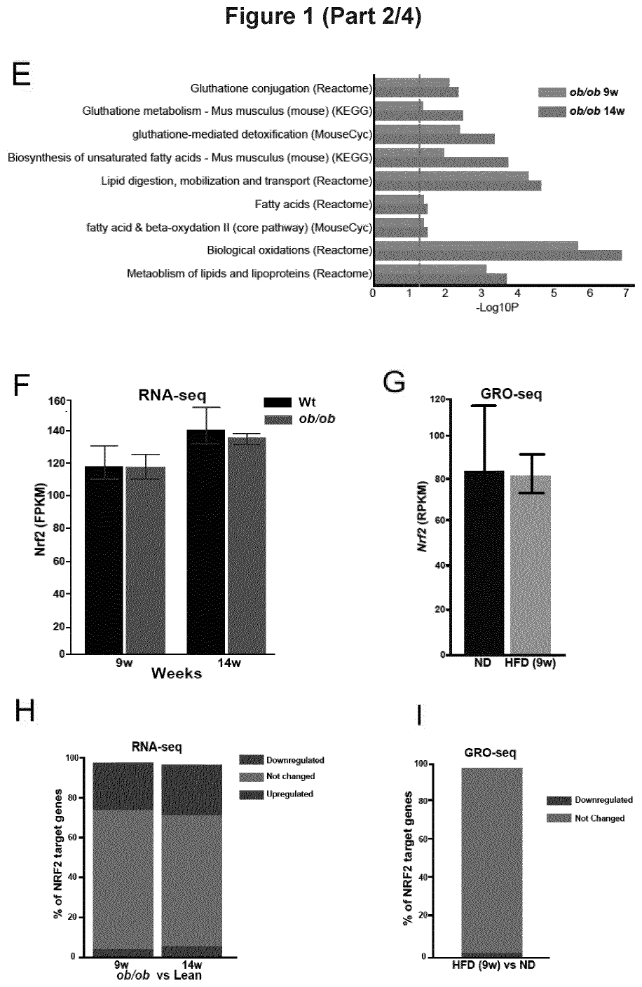

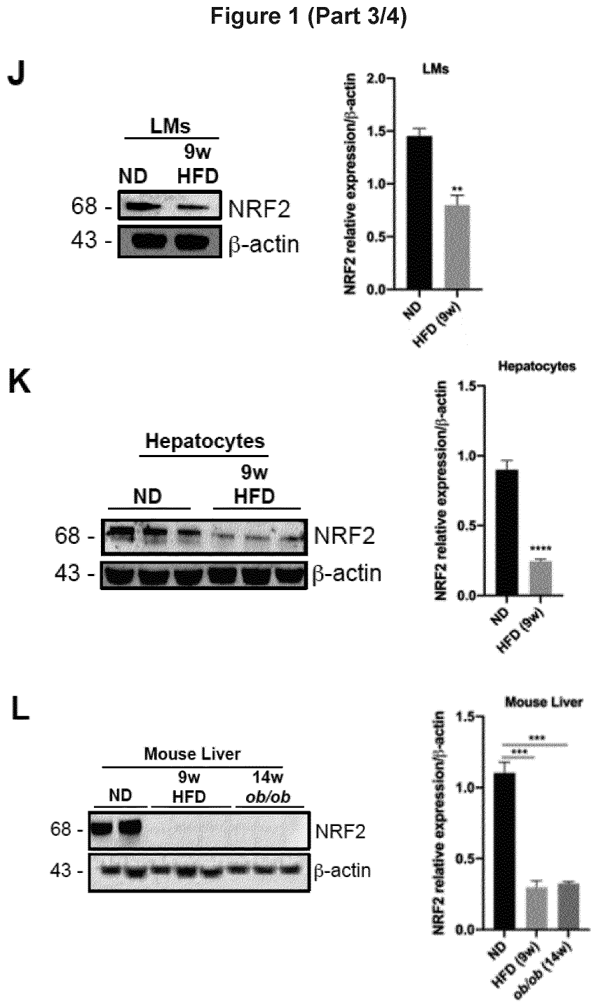

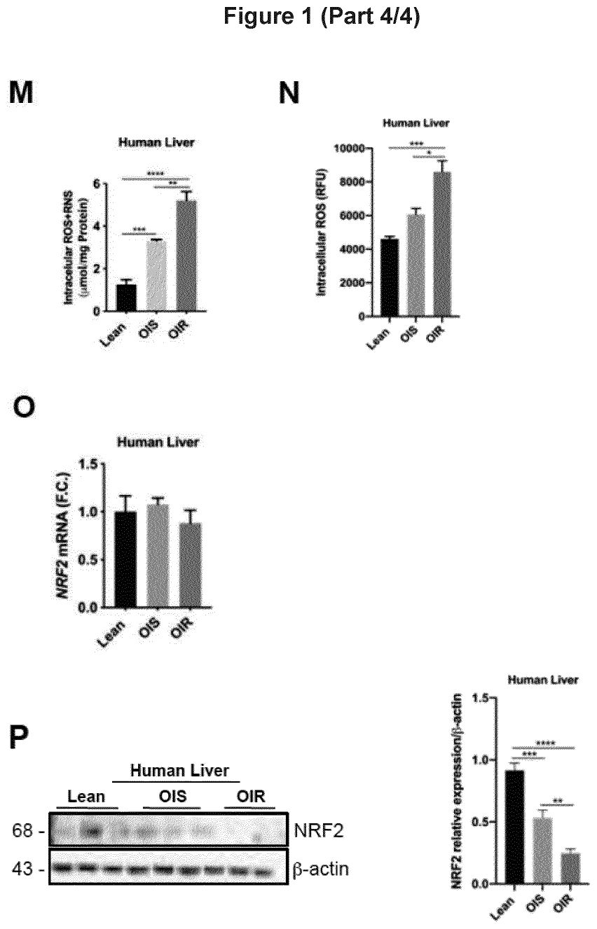

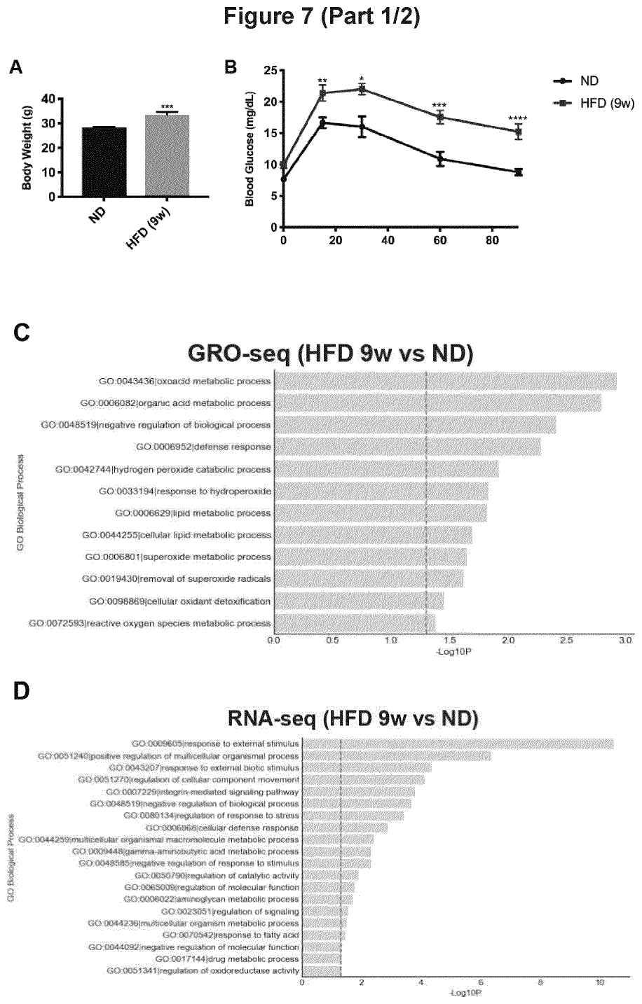

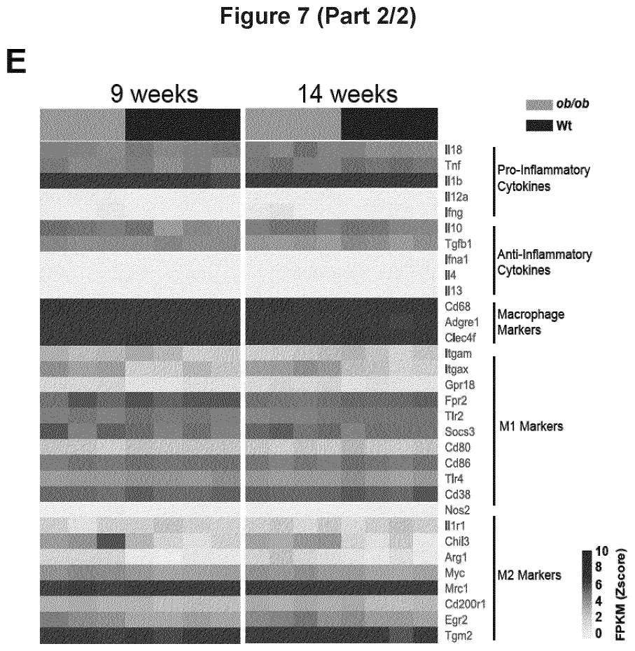

FIG. 1. Oxidative stress in LMs fails to trigger an appropriate antioxidant response in obesity induced insulin resistance. (A) Liver Oil Red O staining of mice fed a HFD or ND for 9 weeks (scale bars, 100 μm); (B) Liver MDA content of mice fed a HFD or ND for 9 weeks (n=4 per group); (C) Extracellular H₂O₂ content on media from LMs of mice fed a HFD or ND for 9 weeks (n=4 per condition); (D-E) Selected significant enriched terms of Gene Ontology (GO) biological processes (D) and enriched pathways (E) from genes differentially expressed in LMs of 9 and 14 weeks ob/ob mice compared to wt (n=4 wt, n=3 ob/ob for 9 weeks; n=4 for 14 weeks); (F-G) Nrf2 mRNA expression data from RNA-seq (F) of 9 and 14-weeks-old wt and ob/ob mice and from GRO-seq (G) of mice fed a HFD or ND for 9 weeks (n=4 wt, n=3 ob/ob, ND, HFD for 9 weeks; n=4 for 14 weeks); (H-I) Percentage of NRF2 target genes significantly up-regulated (‘Upregulated’), down-regulated (‘Downregulated’) or without differential expression (‘Not Changed’) in RNA-seq (H) of 9 and 14 weeks old ob/ob mice compared to wt and in GRO-seq (I) of mice fed a HFD for 9 weeks compared to ND (n=4 wt, ND, HFD, n=3 ob/ob for 9 weeks; n=4 for 14 weeks); (J) WB analysis for NRF2 on LMs from mice fed a HFD or ND for 9 weeks (n=3 per condition); (K) WB analysis for NRF2 on hepatocytes from mice fed a HFD or ND for 9 weeks (n=3 per condition); (L) WB analysis for NRF2 on whole liver from mice fed a HFD or ND for 9 weeks and 14-weeks-old ob/ob mice (n=3 per condition); (M-N) Total intracellular ROS/RNS content (M) and intracellular ROS levels (N) in livers from lean, OIS and OIR human individuals (n=5 per condition); (0) Nrf2 mRNA level measured by RT-qPCR on livers from lean, OIS and OIR human individuals (n=5 per condition). Fold change (F.C.) calculation compared to lean. (P) WB analysis for NRF2 on livers from lean, OIS and OIR human individuals (n=5 per condition). Data are mean values±SEM. All WB quantifications are compared to b-actin levels. **p<0.01, ***p<0.001, **** p<0.0001. See also Table 4 and FIG. 7 (S1).

FIG. 2. The expression of miR-144 is increased in obese LMs and targets the translation of NRF2. (A) WB of NRF2 and KEAP1 after immunoprecipitation of KEAP1 on livers from lean, OIS and OIR human individuals (n=3 per condition). Quantification compared to KEAP-1 levels; (B-C) Heatmaps of significantly and commonly upregulated miRNAs in LMs of 9 (B) and 14 (C) weeks-old ob/ob compared to wt (n=4 wt, n=3 ob/ob for 9 weeks; n=4 per condition for 14 weeks;) (D) Stem-loop RT-qPCR analysis of miR-on LMs from mice fed a HFD or ND for 9 weeks (n=3 per condition); (E) Stem-loop RT-qPCR analysis of miR-144 on livers from mice fed a HFD or ND for 9 weeks and 14-weeks-old ob/ob mice (n=3 per condition); (F) Stem-loop RT-qPCR analysis of miR-144 on livers from lean, OIS and OIR human individuals (n=5 per condition). Data are mean values±SEM. All qPCRs data are fold change (F.C.) compared to ND-fed mice or lean individuals. *p<0.05, **p<0.01, ***p<0.001, **** p<0.0001. RPM, Reads per Million Reads. See also FIG. 8 (S2).

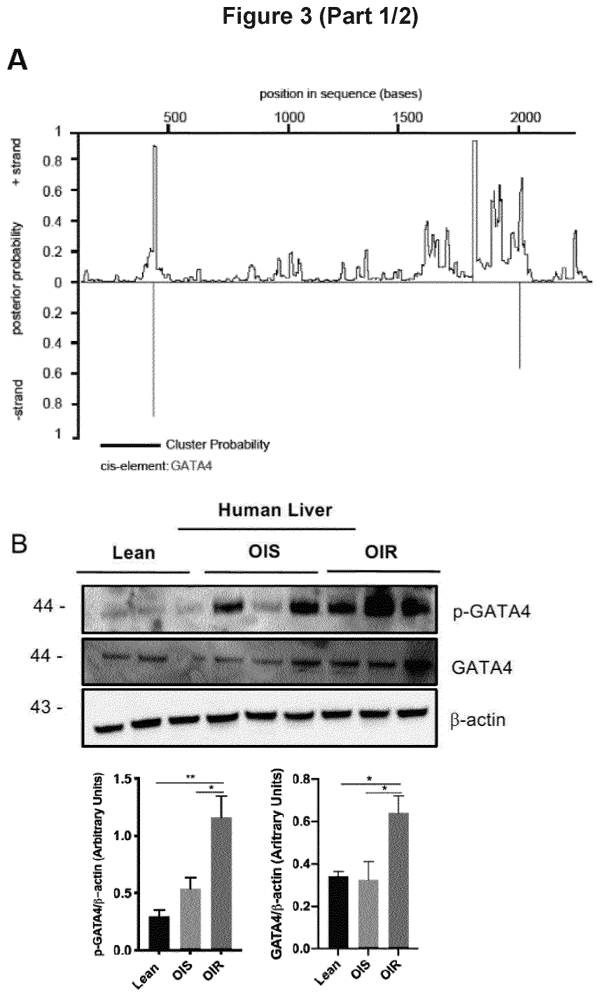

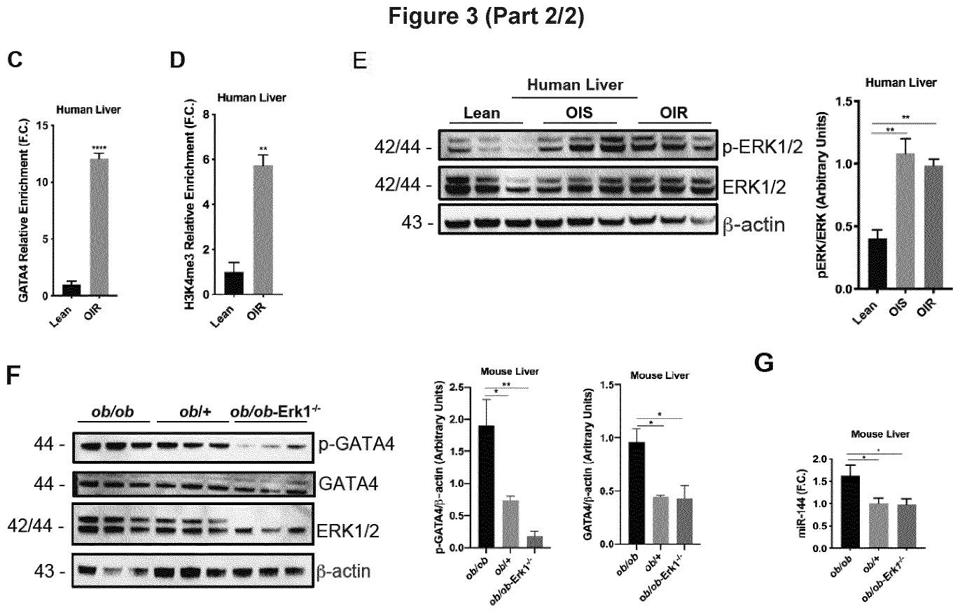

FIG. 3. The transcription factor GATA4 drives the expression of miR-144 in liver of insulin resistant patients. (A) In silico prediction analysis for GATA4 binding domains on miR-144237 promoter region (software CISTER); (B) WB analysis showing phosphorylated-GATA4 and GATA4 in livers from lean, OIS and OIR human individuals (n=3 per condition). Quantification compared to b-actin levels; (C-D) ChIP-qPCR analysis of GATA4 and H3K4me3 relative enrichment on miR-144 promoter of livers from lean and OIR human individuals (n=3 per condition). Data are Fold Change (F.C.) compared to lean and normalized by IgG. (E) WB analysis showing phosphorylated-ERK1/2 and ERK1/2 on livers from lean, OIS and OIR human individuals (n=3 per condition). Quantification of p-ERK1/2/ERK1/2 ratio; (F) WB analysis of p-GATA4, GATA4 and ERK1/2 on livers from ob/+, ob/ob and ob/ob-Erk1−/− mice (n=7 per condition). Quantification compared to b-actin levels; (G) Stem-loop RT-qPCR analysis of miR-144 on livers from ob/+, ob/ob and ob/ob−Erk1−/− mice (n=7 per condition). Data are fold change (F.C.) compared to ob/+. Data are mean values±SEM. *p<0.05, **p<0.01, **** p<0.0001.

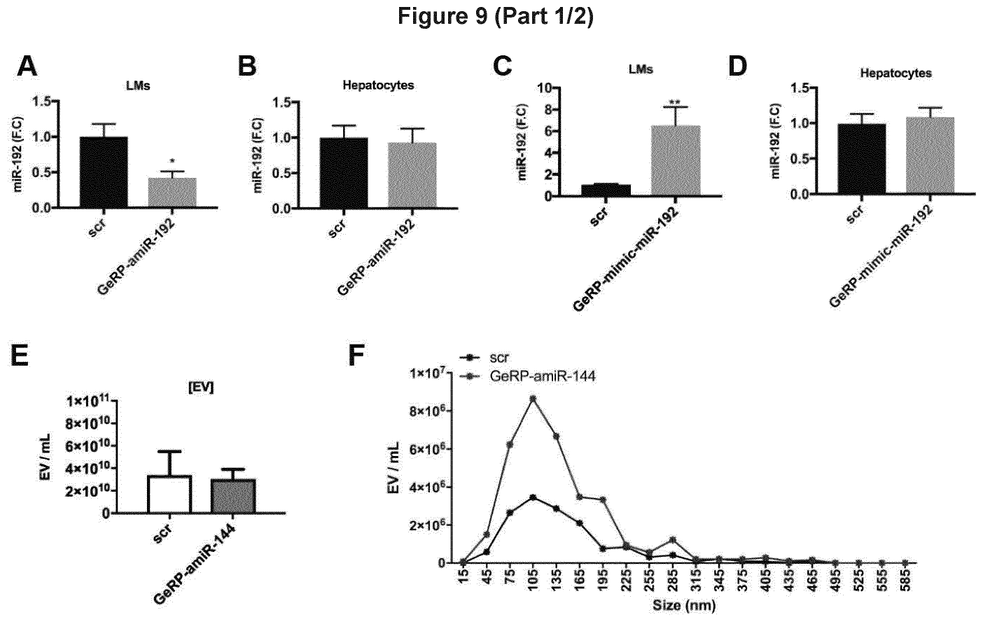

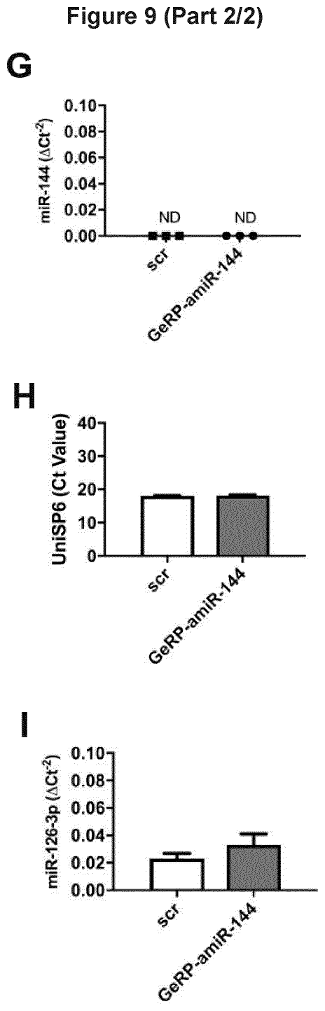

FIG. 4. Silencing miR-144 in liver macrophages reduces ROS release and leads to a decreased expression of miR-144 in hepatocytes. (A) Protocol of GeRP-amiR-144 treatment; (B-C) Stem-loop RT-qPCR analysis of miR-144 on LMs (B) and hepatocytes (C) from scrambled and GeRP-amiR-144-treated mice (n=4 per condition); (D) Stem-loop RT-qPCR analysis of miR-532 on livers, LMs and hepatocytes from scrambled and GeRP-amiR-144-treated mice (n=4 per condition); (E-F) Extracellular H₂O₂ content on media from LMs (E) and hepatocytes (F) from scrambled and GeRP-amiR-144-treated mice (n=4 per condition); (G) WB analysis of p-GATA4 and GATA4 on hepatocytes from scrambled and GeRP-amiR-144-treated mice (n=3 per condition). Quantification of p-GATA4/GATA4 ratio; (H) Stem-loop RT-qPCR analysis of miR-144 on human NPCs exposed to H₂O₂ and treated with scrambled and GeRP-amiR-144 (n=3 per condition); (I) RT-qPCR analysis of GATA4 on human NPCs exposed to H₂O₂ and treated with scrambled and GeRP-amiR-144 (n=3 per condition); (J) RT-qPCR analysis of NRF2 target genes NQO1, GSTP1 and CES2G on human NPCs exposed to H₂O₂ and treated with scrambled and GeRP-amiR-144 (n=3 per condition); (K) Extracellular H₂O₂ content on media from human NPCs exposed to H₂O₂ and treated with scrambled and GeRP-amiR-144 (n=3 per condition); (L) Stem-loop RT-qPCR analysis of miR-144 on hepatocytes (Heps) from human liver spheroids exposed to H₂O₂ and treated with scrambled and GeRP-amiR-144 (pooled liver organoids from 1 human donor); (M) Schematic representation of liver spheroids cell types proportion and treatments; (N) Stem-loop RT-qPCR analysis of miR-144 on hepatocytes (Heps) and NPCs from human liver spheroids exposed to FFA and treated with scrambled and amiR-144 (pooled liver spheroids from 1 human donor); (0) RT-qPCR analysis of NRF2 target genes NQO1, GSTP1 and CES2G on hepatocytes (Heps) from human liver spheroids exposed to FFA and treated with scrambled and amiR-144 (pooled liver spheroids from 1 human donor). Data are mean values±SEM. All qPCRs data fold change (F.C.) compared to scr. *p<0.05, **p<0.01, **** p<0.0001. See also FIG. 9 (S3).

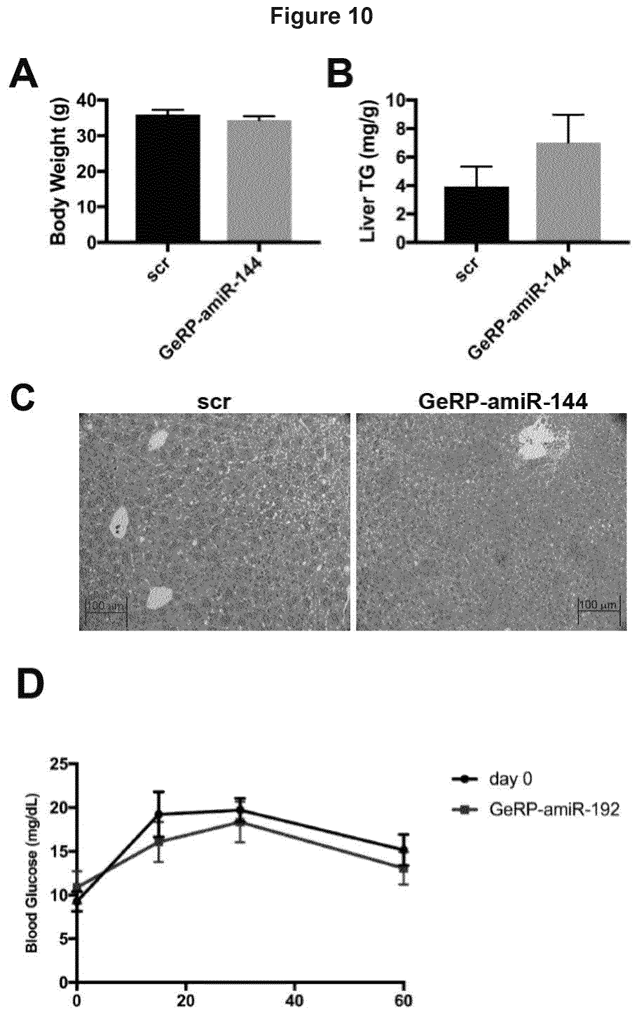

FIG. 5. Silencing miR-144 in LMs reduces oxidative stress and improves hepatic metabolism in insulin resistance. (A-C) WB analysis of NRF2 on livers (A), LMs (B) and hepatocytes (C) from scrambled and GeRP-amiR-144-treated mice (n=4 per condition); (D-E) RT-qPCR analysis of NRF2 target genes: Nqo1, Gstp1 and Ces2G on LMs and hepatocytes from scrambled and GeRP-amiR-144-treated mice (n=4 per condition); (F) Total intracellular ROS/RNS content in livers from scrambled and GeRP-amiR-144-treated mice (n=4 per condition); (G-H) Intracellular ROS (G) and RNS (H) levels in hepatocytes from scrambled and GeRP-amiR-144-treated mice (n=4 per condition); (I) Percentage of resident and recruited macrophages from scrambled and GeRP-amiR-144-treated ND-fed mice (n=4 per condition); (J) Stem-loop RT-qPCR analysis of miR-144 on CD45+/F4/80+/Cd11b+/FITC+ LMs from scrambled and GeRP-amiR-144-treated ND-fed mice (n=4 per condition); (K) Stem-loop RT-qPCR analysis of miR-144 on CD45+/F4/80+/Cd11b+/FITC− LMs from scrambled and GeRP-amiR-144-treated ND-fed mice (n=4 per condition); (L) Stem-loop RT-qPCR analysis of miR-144 on CD45−/FITC− NPCs from scrambled and GeRP-amiR-144-treated ND-fed mice (n=4 per condition); (M) Stem-loop RT-qPCR analysis of miR-144 on hepatocytes from scrambled and GeRP-amiR-144-treated ND-fed mice (n=4 per condition); (N) Transmission Electron Microscopy showing increased number of mitochondria in livers from scrambled and GeRP-amiR-144-treated mice (n=2 per condition). Black arrows depict mitochondria (scale bars, 500 nm); (O) Number of mitochondria per section in livers from scrambled and GeRP-amiR-144-treated mice (n=20 section per condition; (P) Transmission Electron Microscopy showing increased stored glycogen in livers from scrambled and GeRP-amiR-144 treated mice (n=2 per condition). Black arrows depict glycogen deposits (scale bars, 500 nm); (Q) IP-GTT of scrambled and GeRP-amiR-144-treated mice (n=5 per condition). Data are mean values±SEM. All WB quantifications are compared to b-actin levels. All qPCRs data are fold change (F.C.) compared to scr. *p<0.05, **** p<0.0001. See also FIG. 10 (S4).

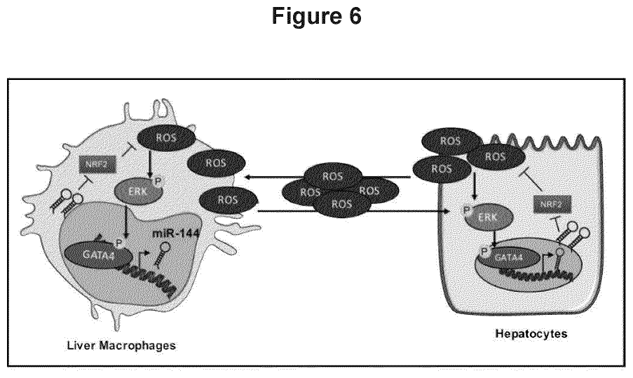

FIG. 6. Model of oxidative stress regulation by LMs in obesity. Excessive lipid accumulation in liver during obesity leads to oxidative stress. LMs exacerbate ROS release without an overt pro-inflammatory phenotype. ROS, as a secondary messenger, activate ERK and GATA4 increasing expression of miR-144, a miRNA targeting NRF2. The subsequent downregulation of NRF2 protein prevents both cell types to engage an appropriate anti-oxidant response.

FIG. 7 (S1). Oxidative stress in LMs fails to trigger an appropriate antioxidant response in obesity-induced insulin resistance. (A) Body weight of mice fed an HFD or ND for 9 weeks (n=10 per condition); (B) IP-GTT of of mice fed an HFD or ND for 9 weeks (n=10 per condition); (C) Gene Onthology (GO) analysis from GRO-seq dataset comparing mice fed an HFD for 9 weeks with ND; (D) Gene Onthology (GO) analysis from RNA-seq dataset comparing mice fed an HFD for 9 weeks with ND. (E) Expression heatmap of pro-inflammatory cytokines, anti-inflammatory cytokines, macrophage, M1 and M2 markers targets in ob/ob mice compared to wt (n=4 wt, n=3 ob/ob for 9 weeks; n=4 per condition for 14 weeks); Data are mean values±SEM. *p<0.05, **p<0.01, ***p<0.001, **** p<0.0001. FPKM, Fragments Per Kilobase Million. Related to FIG. 1.

FIG. 8 (S2). The expression of miR-144 is increased in obese LMs and targets the translation of NRF2. (A) WB of ubiquitin and NRF2 after immunoprecipitation of NRF2 from livers isolated from Lean, OIS and OIR human individuals (n=3 per condition). Densitometry representing NRF2 ubiquitination/NRF2 levels ratio (B) stem-loop RT-qPCR analysis of miR-144 performed on Visceral Adipose Tissue (VAT) from HFD and ND-fed mice (n=3 per condition). Data are expressed as fold change (F.C.) compared to ND-fed; (C) stem-loop RT-qPCR analysis of miR-144 performed on Spleen from HFD and ND-fed mice (n=3 per condition). Data are expressed as fold change (F.C.) compared to ND-fed; (D) stem-loop RT-qPCR analysis of miR-144 performed on Lung from HFD and ND-fed mice (n=3 per condition). Data are expressed as fold change (F.C.) compared to ND-fed. Related to FIG. 2.

FIG. 9 (S3). Silencing miR-144 in LMs reduces ROS release and leads to a decreased expression of miR-144 in hepatocytes. (A) stem-loop RT-qPCR analysis of miR-192 performed on LMs isolated from scrambled and GeRP-amiR-192 treated mice (n=4 per condition). Data are expressed as fold change (F.C.) compared to scr; (B) stem-loop RT-qPCR analysis of miR-192 performed on hepatocytes isolated from scrambled and GeRP-amiR-192 treated mice (n=4 per condition). Data are expressed as fold change (F.C.) compared to scr; (C) stem-loop RT-qPCR analysis of miR-192 performed on LMs isolated from scrambled and GeRP-mimic-miR-192 treated mice (n=4 per condition). Data are expressed as fold change (F.C.) compared to scr; (D) stem-loop RT-qPCR analysis of miR-192 performed on hepatocytes isolated from scrambled and GeRP-mimic-miR-192 treated mice (n=4 per condition); (E) Concentration of EV isolated from media of LMs isolated from scrambled and GeRP-amiR-144 treated mice (n=4 per condition); (F) EV content in media collected from LMs isolated from scrambled and GeRP-amiR-144 treated mice (n=4 per condition); (G) stem-loop RT-qPCR analysis for miR-144 on EV isolated from media of LMs isolated from scrambled and GeRP-amiR-144 treated mice (n=4 per condition). DCt-2 values; (H) stem-loop RT-qPCR analysis for UNISP6 on EV isolated from media of LMs isolated from scrambled and GeRP-amiR-144 treated mice (n=4 per condition). Ct values; (I) stem-loop RT-qPCR analysis for miR-126 on EV isolated from media of LMs isolated from scrambled and GeRP-amiR-144 treated mice (n=4 per condition). DCt-2 values. Data are mean values±SEM. **p<0.01. Related to FIG. 4.

FIG. 10 (S4). Silencing miR-144 in LMs reduces oxidative stress and improves hepatic metabolism in insulin resistance. (A) Body Weight of scrambled and GeRP-amiR-144 treated mice (n=10 per condition) (B) Liver triglycerides content of scrambled and GeRP-amiR-144 treated mice (n=4 per condition); (C) H&E staining of livers from scrambled and GeRP-amiR-144 treated mice (scale bar, 100 μm); (D) IP-GTT in GeRP-amiR-192 treated mice. Day 0 vs Day7 of treatment. (n=4 per condition). Related to FIG. 5.

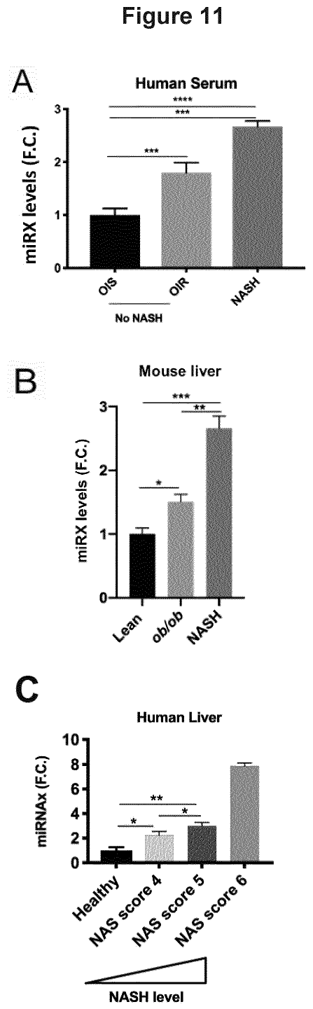

FIG. 11. (A) Stem-loop RT-qPCR analysis of miR-144 expression performed on human serum from OIS, OIR and NASH human individuals (n=6 OIR, OIS; n=5 NASH) (B) stem-loop RT-qPCR analysis of miR-144 expression performed in lean and ob/ob mice fed an ND diet or ob/ob mice fed a diet high in trans-fat (40%), fructose (22%) and cholesterol (2%) for 12 weeks which induces NASH (n=3 for lean and ob/ob and n=5 for ob/ob with NASH). (C) stem-loop RT-qPCR analysis of miR-144 expression performed in liver biopsies collected from healthy and obese patients affected by NASH. NAS index score is a marker for different stage of NASH. NAS index was scored by pathologists at Universitätsklinikum Hamburg-Eppendorf. n=5 for Healthy, n=8 for NAS score 4, n=4 for NAS score 5 and n=2 for NAS score 6. Data are mean values±SEM. *p<0.05, **p<0.01, ***p<0.001.

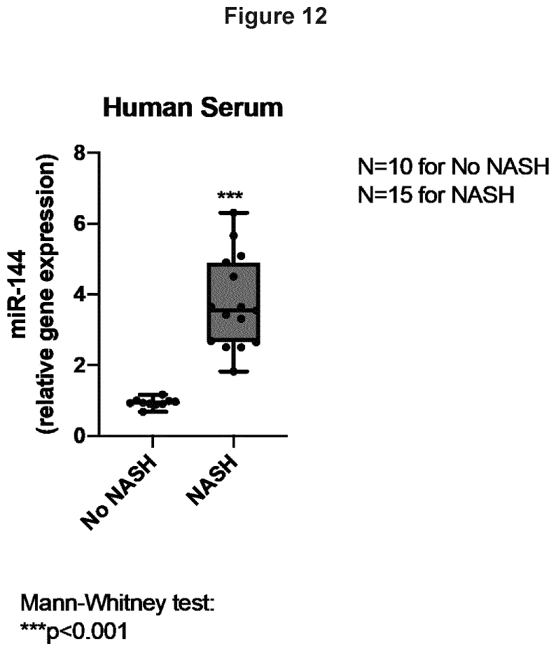

FIG. 12. Stem-loop RT-qPCR analysis of miR-144 expression performed on human serum from human individual with NASH or with no NASH (n=10 no NASH; n=15 NASH). Data are mean values±SEM. ***p<0.001.

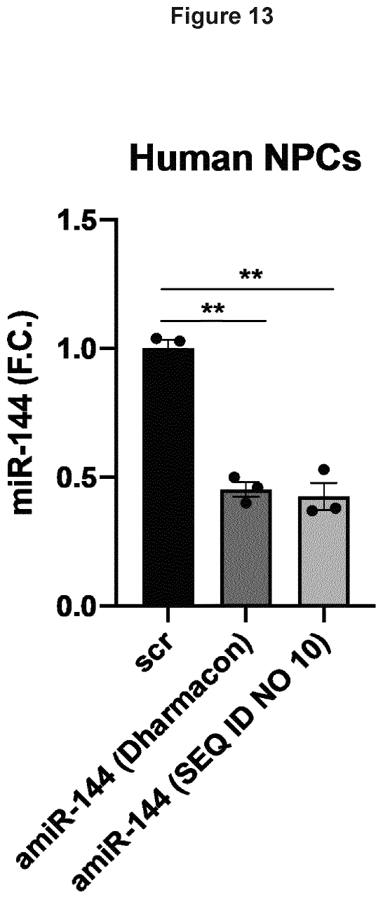

FIG. 13. Stem-loop RT-qPCR analysis of miR-144 on human NPCs exposed to FFA and treated with scrambled, amiR-144 (Dharmacon; IH-300612-06) and amiR-144 (SEQ ID NO10). (n=3 per condition). Data are expressed as fold change (F.C.) compared to scr. Data are mean values±SEM. **p<0.01.

### EXAMPLE 1—LIVER MACROPHAGES INHIBIT THE ENDOGENOUS ANTIOXIDANT RESPONSE IN OBESITY-ASSOCIATED INSULIN RESISTANCE

### Summary

Liver Macrophages Exacerbate Oxidative Stress Induced by Hepatic Steatosis in Obesity by Blocking the Endogenous Anti-Oxidant Response.

Obesity and insulin resistance are risk factors for non-alcoholic fatty liver disease (NAFLD), the most common chronic liver disease worldwide. Since no approved medication nor an accurate and non-invasive diagnosis are currently available for NAFLD, there is a clear need for better understand the link between obesity and NAFLD. Lipids accumulation during obesity is associated with oxidative stress and inflammatory activation of liver macrophages (LMs). However, we show that while LMs do not become pro-inflammatory, they display signs of oxidative stress. In the livers of both humans and mice, levels of antioxidant nuclear factor erythroid 2-related factor 2 (NRF2) were downregulated with obesity and insulin resistance, yielding an impaired response to lipid accumulation. At the molecular level, a microRNA targeting NRF2, miR-144, was elevated in the livers of obese insulin resistant humans and mice, and specific silencing of miR-144 in LMs was sufficient to rescue NRF2 and the antioxidant response. These results highlight the pathological role of LMs and their therapeutic potential restore the impaired endogenous antioxidant response in obesity-associated NAFLD.

### Introduction

Obesity represents a major health issue worldwide as excessive weight significantly increases the risk for several metabolic complications including non-alcoholic fatty liver disease (NAFLD), insulin resistance and type 2 diabetes (T2D) (1, 2). Given its major role in the metabolism of nutrients, the liver plays a central role in the control of metabolic homeostasis (3).

Fatty liver is the result of excessive lipid accumulation due to a lower fat storage capacity of adipose tissue in obesity-associated insulin resistance (4). The inability of the liver to handle this overload of fat leads to aberrant lipid peroxidation and excessive production of Reactive Oxygen Species (ROS)/Reactive Nitrogen Species (RNS) (5). ROS and RNS are thought to trigger the phenotypic switch of liver macrophages (LMs) from anti-inflammatory (M2) to a pro-inflammatory activation state (M1), leading to insulin resistance (6, 7). However, we have recently discovered that LMs do not become inflammatory in obesity-induced insulin resistance (8). We have also demonstrated that LMs produce non-inflammatory factors able to regulate insulin sensitivity. Nevertheless, while LMs did not become inflammatory they did display signs of oxidative stress. Indeed, transcriptomic profiling showed that several metabolic pathways involved in ROS/RNS production, such as the tricarboxylic acid cycle (TCA) cycle and Oxidative Phosphorylation (OXPHOS), were significantly regulated in LMs of obese compared to control mice (8). We therefore hypothesize that LMs may regulate oxidative stress independently of their inflammatory status in obese-induced insulin resistance.

Nuclear factor erythroid 2-related factor 2 (NFE2L2/Nrf2), a basic leucine zipper transcription factor, is as a master regulator of redox homeostasis (9). Under normal physiological conditions, NRF2 is targeted to proteasomic degradation through its association with Kelch-like ECH-associated protein-1 (KEAP1). Conversely, upon oxidative stress this complex dissociates and NRF2 translocates to the nucleus where it binds to the antioxidant responsive element (ARE), thus driving the antioxidant response. The antioxidant capacity of the liver is reduced in obesity, although the molecular mechanisms underlying this impairment remain unknown (10).

Herein we report that NRF2 protein levels were dramatically decreased in the livers of obese insulin resistant humans and mice compared with healthy controls. Interestingly, NRF2 transcription, NRF2-KEAP1 interaction and NRF2 ubiquitination all remained unchanged in insulin resistant condition, suggesting that other post-transcriptional mechanisms may impact NRF2 levels. We identified miR-144 as a potent regulator of NRF2 protein levels in obesity-induced insulin resistance in mice and humans. Unexpectedly, using a unique method to specifically silence genes in LMs in vivo (11, 12), we found that selective silencing of miR-144 was sufficient to decrease ROS and RNS release by LMs and hepatocytes and eventually accumulation in the whole liver through the rescue of NRF2 in obese mice. Altogether, our data demonstrate that, in obesity-induced insulin resistance characterized by excessive hepatic lipid accumulation, LMs produce miRNAs able to impair the antioxidant capacity of the liver which are not linked to activation of pro-inflammatory pathways.

Results

Oxidative Stress in LMs Fails to Trigger an Appropriate Antioxidant Response in Obesity-Induced Insulin Resistance

Increased lipid peroxidation products are a common marker of oxidative stress. To confirm that High-Fat Diet (HFD)-induced obesity is associated with increased lipid peroxidation in the liver, levels of malondialdehyde (MDA), a reactive aldehyde produced during lipid peroxidation, were measured. As expected, in mice fed a HFD for 9 weeks, there was a significant increase in weight and impaired glucose handling (FIG. 7 (S1)A-B) coupled with lipid accumulation and significantly increased MDA levels in the liver (FIG. 1A-B). In addition, ROS release in the media of LMs isolated from obese mice fed HFD was dramatically increased compared to controls (FIG. 10).

To study the phenotype of LMs in obesity we analyzed their transcriptomic profile in obese mice (ob/ob mice) at two different ages (9 and 14-weeks-old) and compared to their age-matched1 wildtype (wt) lean controls. Gene Ontology (GO) enrichment analysis revealed oxidative stress as one of the most dysregulated biological processes in obesity (FIG. 1D). In addition, pathway analysis revealed a significant impairment in the lipid oxidation and antioxidant response pathways in obese compared with lean mice (FIG. 1E). Similar findings were observed in a diet-induced model of obesity where pathways involved in oxidative stress were enriched in the LMs of mice fed a high fat diet (HFD) for 9 weeks compared to in lean controls fed a normal diet (FIG. 7 (S1)C-D). Consistent with our previous findings (8), transcriptomic profiling failed to reveal a proinflammatory phenotypic switch in liver macrophages in obesity (FIG. 7 (S1)E).

Since oxidative stress is known to trigger the endogenous antioxidant response under the control of the transcription factor NRF2, we measured its expression in LMs of ob/ob mice. We observed that the mRNA expression of Nrf2 was not changed in LMs of obese mice compared to lean mice (FIG. 1F). As RNA-seq only measures steady-state transcript levels we also performed global run-on sequencing (GRO-seq) which allows the measurement of nascent transcripts. This revealed that Nrf2 transcription remained unchanged in HFD (FIG. 1G). RNA-seq and GRO-seq analysis indicated that most of the NRF2 target genes remained unchanged in obesity despite the increased levels of ROS in LMs (FIG. 1H-I, complete list in Table 3 (S9)).

TABLE 3 (S9)
Differentially expressed genes from the comparison of
HFD vs. ND mice (RNA-seq) enriched in inflammatory response
GO biological process (GO:0006954).
Genes were selected based on FDR < 0.05 and |log2FoldChange| > 1.

| SYMBOL | GENENAME | log2FoldChange | pvalue | FDR |
|---|---|---|---|---|
| Xcl1 | chemokine (C motif) | −1.668983084 | 2.30498E−07 | 5.71443E−05 |
| | ligand 1 | | | |
| Slamf1 | signaling lymphocytic | −1.176154012 | 7.322E−06 | 0.00075306 |
| | activation molecule | | | |
| | family member 1 | | | |
| Tlr5 | toll-like receptor 5 | −1.126812892 | 0.000980469 | 0.021079631 |
| Il2ra | interleukin 2 receptor, | −1.670551771 | 8.8501E−06 | 0.000856229 |
| | alpha chain | | | |
| Dab2ip | disabled 2 interacting | 1.004758096 | 9.34995E−08 | 3.00714E−05 |
| | protein | | | |
| Tnfrsf8 | tumor necrosis factor | −1.063404878 | 0.00250234 | 0.039311811 |
| | receptor superfamily, | | | |
| | member 8 | | | |
| Tnfrsf9 | tumor necrosis factor | −1.339507834 | 0.000564433 | 0.014585773 |
| | receptor superfamily, | | | |
| | member 9 | | | |
| Tnfrsf25 | tumor necrosis factor | −1.176119114 | 1.93026E-06 | 0.000290314 |
| | receptor superfamily, | | | |
| | member 25 | | | |
| Tnfrsf18 | tumor necrosis factor | −1.404131917 | 1.96964E−05 | 0.001478784 |
| | receptor superfamily, | | | |
| | member 18 | | | |
| Cxcl13 | chemokine | −1.462827958 | 0.000573904 | 0.014766387 |
| | (C-X-C motif) | | | |
| | ligand 13 | | | |
| Il5ra | interleukin 5 receptor, | −1.198387132 | 0.000967774 | 0.020958165 |
| | alpha | | | |
| Alox5 | arachidonate | −1.321128279 | 0.001196058 | 0.024007563 |
| | 5-lipoxygenase | | | |
| Cd27 | CD27 antigen | −1.066829743 | 0.000265147 | 0.008656389 |
| Ffar2 | free fatty acid | −1.01221227 | 4.79568E−05 | 0.002740923 |
| | receptor 2 | | | |
| Ffar3 | free fatty acid | −1.551958057 | 0.000426068 | 0.01202896 |
| | receptor 3 | | | |
| Saa3 | serum amyloid A 3 | −1.174157176 | 0.003579088 | 0.049668969 |
| Calca | calcitonin/calcitonin- | −1.144979342 | 3.25396E−05 | 0.002125618 |
| | related polypeptide, | | | |
| | alpha | | | |
| Lat | linker for activation | −1.420479096 | 1.41135E−05 | 0.001182755 |
| | of T cells | | | |
| Itgam | integrin alpha M | −1.340434674 | 0.000654689 | 0.016146725 |
| Itgam | integrin alpha M | −1.332973574 | 0.00079883 | 0.018351501 |
| Pstpip1 | proline-serine-threonine | −1.377683557 | 2.13952E−05 | 0.001567417 |
| | phosphatase- | | | |
| | interacting protein 1 | | | |
| Ccr4 | chemokine (C-C motif) | −1.836974143 | 5.1077E−09 | 3.03908E−06 |
| | receptor 4 | | | |
| Cxcr6 | chemokine (C-X-C | −1.549954712 | 2.71276E−06 | 0.000377566 |
| | motif) receptor 6 | | | |
| Ccr1l1 | chemokine (C-C motif) | −1.225133064 | 0.002154444 | 0.03553415 |
| | receptor 1-like 1 | | | |
| Ccr3 | chemokine (C-C motif) | −1.857010661 | 2.49594E−14 | 1.48508E−10 |
| | receptor 3 | | | |
| Ccr2 | chemokine (C-C motif) | −1.390856296 | 0.000158232 | 0.006031436 |
| | receptor 2 | | | |
| Cd24a | CD24a antigen | −1.412707188 | 0.000105578 | 0.004653234 |
| P2rx1 | purinergic receptor | −1.31837306 | 0.001657487 | 0.029803311 |
| | P2X, ligand-gated ion | | | |
| | channel, 1 | | | |
| Cd9 | chemokine (C-C motif) | −1.27087659 | 0.002319404 | 0.037424954 |
| | ligand 9 | | | |
| Ccr7 | chemokine (C-C motif) | −1.462271584 | 0.000229443 | 0.007857183 |
| | receptor 7 | | | |
| Mefv | Mediterranean fever | −1.515823259 | 1.93949E−06 | 0.000290314 |
| Ccl96 | CD96 antigen | −1.662553173 | 9.30292E−07 | 0.000172976 |
| Ccr6 | chemokine (C-C motif) | −1.553326766 | 3.76346E−05 | 0.00233256 |
| | receptor 6 | | | |
| Fpr2 | formyl peptide | −1.059978268 | 0.00314226 | 0.045601094 |
| | receptor 2 | | | |
| Lta | lymphotoxin A | −1.202699402 | 7.08196E−05 | 0.003589664 |
| Cd6 | CD6 antigen | −1.58831069 | 4.53023E−06 | 0.000547307 |
| Cd40lg | CD40 ligand | −1.464669233 | 2.32618E−05 | 0.001667562 |
| Cxcr3 | chemokine (C-X-C | −1.148578713 | 0.000104586 | 0.004618102 |
| | motif) receptor 3 | | | |
| Acod1 | aconitate | −1.20837213 | 0.000228958 | 0.007851861 |
| | decarboxylase 1 | | | |

Interestingly, while Nrf2 mRNA levels and transcription remained unchanged upon inducing obesity, we observed a dramatic decrease of NRF2 protein levels in LMs of HFD-fed mice compared to controls (FIG. 1J). In addition, NRF2 protein levels were decreased in both hepatocytes and the whole liver of HFD-fed mice compared to controls (FIG. 1K-L). This decrease in NRF2 protein levels was also observed in livers of ob/ob mice compared to wt controls (FIG. 1L). Therefore, the antioxidant response driven by NRF2 is impaired in LMs and hepatocytes from both HFD and genetically induced obesity.

To test whether the impaired antioxidant response observed in murine obese livers also occurred in humans we measured oxidative stress and NRF2 mRNA and protein levels in lean, obese insulin sensitive (OIS) and insulin resistant (OIR) individuals (Table 4). First, a significantly higher ROS and RNS accumulation was observed in the livers of obese patients compared to lean individuals, which was exacerbated with insulin resistance and confirmed the association between liver oxidative stress, obesity and insulin resistance (FIG. 1M-N). Similar to mice, while there was no change in NRF2 mRNA levels (FIG. 10), the NRF2 protein levels were dramatically decreased in OIR compared to OIS and lean individuals (FIG. 1P).

Taken together these results demonstrated that oxidative stress fails to induce an NRF2-mediated antioxidant response during obesity-associated insulin resistance in mice and human livers.

TABLE 4
Parameters of Human Obese Insulin Sensitive (OIS) and Obese
Insulin Resistant (OIR) individuals selected in the study. HOMA,
homeostatic model assessment; Insulin sensitivity is assessed
by HOMA-IR method. HIS: Hepatic Steatosis Index.

| Code | Sex | Age | BMI | HOMA-IR | HSI | |
|---|---|---|---|---|---|---|
| 6127 | M | 43 | 37.2 | 0.56 | 46.3 | OIS |
| 6006 | M | 43 | 40.8 | 1.1 | 51 | OIS |
| 6108 | M | 40 | 39.2 | 1.56 | 49.8 | OIS |
| 6141 | M | 54 | 35.8 | 1.69 | 50 | OIS |
| 6165 | M | 42 | 34.7 | 1.88 | 42.5 | OIS |
| 6168 | M | 25 | 40.3 | 6.18 | 53.5 | OIR |
| 6157 | M | 44 | 36.6 | 6.99 | 42.5 | OIR |
| 6027 | M | 38 | 39.8 | 10.65 | 55.6 | OIR |
| 5870 | M | 37 | 37.7 | 10.94 | 45.6 | OIR |
| 6029 | M | 64 | 39.6 | 13.28 | 47.2 | OIR |

The Expression of miR-144 is Increased in Obese LMs and Targets the Translation of NRF2

We then investigated the mechanism(s) whereby NRF2 protein levels were decreased with insulin resistance. Since the transcription of NRF2 was unaffected with obesity and KEAP1 is an important regulator of NRF2 through its ubiquitination and degradation via the proteasome, we investigated KEAP1-NRF2 interaction in lean, OIS and OIR livers.

Surprisingly, despite unaltered levels of KEAP1 protein in OIS and OIR livers, the levels of NRF2 associated to KEAP1 were strongly reduced in OIR condition (FIG. 2A). Moreover, NRF2 ubiquitination levels remained unchanged (FIG. 8 (S2)A). These data suggested that the decrease in NRF2 was not the consequence of only its ubiquitination but was likely due to an alternative post-transcriptional mechanism independent of KEAP1-induced degradation. Given that NRF2 was downregulated in LMs and miRNAs are known to regulate both transcription and translation, we analyzed the LMs miRNome in obese animals. We performed small-RNA sequencing on LMs of 9- and 14-week-old ob/ob mice and age-matched wt controls. Six miRNAs were significantly and commonly upregulated with obesity, in ob/ob mice compared to matched controls (FIG. 2B-C). Using a predictive in silico database (miRwalk2.0) (13), we noted that NRF2 was a validated target of miR-144, one of these upregulated miRNAs.

We therefore performed stem-loop RT-qPCR analysis in the two models of obesity. We found miR-144 expression increased in isolated LMs of HFD mice (FIG. 2D), as well as in livers of both HFD-fed and ob/ob mice compared to their respective controls (FIG. 2E). The observed increase of miR-144 was liver specific, as its expression remained unchanged in the spleen, lung and visceral adipose tissue (VAT) of obese mice (FIG. 8 (S2)B-D). Furthermore, miR-144 expression was significantly increased in livers of OIR compared to lean or OIS individuals (FIG. 2F).

Altogether, these data suggest that miR-144 may mediate the decrease in NRF2 protein levels in obesity-associated insulin resistance in both murine and human livers.

The Transcription Factor GATA4 Drives the Expression of miR-144 in the Liver of Insulin Resistant Patients

To investigate the mechanism triggering the increase of miR-144 in insulin resistance, we performed targeted in silico analysis of its promoter region. Cis-element Cluster Finder (CISTER) software identified a high density of GATA4 binding domains on the miR-144 promoter and enhancer regions (FIG. 3A). This finding prompted us to analyze whether the total and/or phosphorylated levels of the LM-expressed isoform GATA4 were altered. In liver protein lysates from OIR subjects, both GATA4 phosphorylation and protein levels were significantly higher than in OIS and lean individuals (FIG. 3B). To test the hypothesis that GATA4 induces the expression of miR-144 in insulin resistance, we performed Chromatin Immunoprecipitation (ChIP) and analyzed the specific binding of GATA4 to the miR-144 promoter region. ChIP analysis confirmed GATA4 increased binding to the miR-144 promoter in OIR compared to lean individuals (FIG. 3C). We also observed higher levels of H3K4me3 modification, a well-known marker for active transcription, in the insulin resistant condition (FIG. 3D), suggesting an active transcription of the miR-144 locus.

Since oxidative stress is known to activate the ERK pathway, leading to the activation of GATA4 by phosphorylation (14), we next measured ERK1/2 activity in our human cohort.

We observed that ERK1/2 phosphorylation was increased with obesity but to similar degrees in IR and IS individuals (FIG. 3E). We then took advantage of the ob/ob-Erk1−/− mouse model (15) to validate the role of the ERK pathway in the regulation of GATA4 activity in obesity. WB analysis showed a considerable decrease of phosphorylated-GATA4 in the ob/ob-Erk1−/− condition compared to ob/ob and ob/+ insulin sensitive control mice (FIG. 3F). Consistently, miR-144 transcription levels were also decreased in the livers of ob/ob-Erk1−/− mice as well as in ob/+ insulin sensitive mice, as expected (FIG. 3G).

These data strongly suggested that miR-144 expression is controlled by GATA4, which is activated by ERK in insulin resistant mice and humans.

Silencing miR-144 in Liver Macrophages Reduces ROS Release and Leads to a Decreased Expression of miR-144 in Hepatocytes

To investigate the role of miR-144 in the regulation of NRF2 in vivo we took advantage of the Glucan encapsulated RNAi Particle (GeRP) technology (11, 12). GeRPs deliver siRNA and silence genes specifically in LMs without affecting gene expression in other cells of the liver or the rest of the body. Mice were fed a HFD for 7 weeks and then treated with GeRPs containing an antagomiR targeting miR-144 (amiR-144) or a non-targeting control (scr) (See protocol in FIG. 4A). LMs and hepatocytes were isolated and the expression of miR-144 was measured by RT-qPCR. Surprisingly, we observed a significant knockdown of miR-144 in both LMs and hepatocytes (FIG. 4B-C). We addressed the specificity of the GeRP-mediated silencing of miR-144 by measuring the expression of another miRNA, miR-532, which remained unchanged upon treatment with GeRP-amiR-144 (FIG. 4D).

We next addressed whether the silencing of miR-144 observed in hepatocytes following treatment with GeRP-amiR-144 was specific to this particular miRNA or was a general mechanism affecting all miRNAs. We thus treated mice with GeRPs loaded with an antagomir targeting another miRNA, miR-192. Treatment with GeRP-amiR-192 significantly decreased miR-192 expression in LMs but had no effect on hepatocytes (FIG. 9 (S3) A-B). Furthermore, GeRP-mediated delivery of a miR-192 mimic did not affect hepatocytes, whereas it increased miR-192 expression in LMs (FIG. 9 (S3)C-D). Since GeRP-mediated silencing of miR-144 in hepatocytes seemed to be specific to this miRNA, we hypothesized that miR144 could be delivered from LMs to hepatocytes through extracellular vesicles (EV). EV delivery could potentially explain why silencing miR-144 in LMs led to a decreased expression of miR-144 in hepatocytes. To test this hypothesis, we isolated EV from media of LMs silenced with antagomiR-144 and measured the levels of miR-144. While control miRNAs (miR-126-3p and UNISP6) were present in EVs from LMs media, miR-144 was undetectable (FIG. 9 (S3) E-I). The alternative explanation for the decreased expression of miR-144 in hepatocytes following silencing in LMs was a reduction of extracellular ROS levels which would no longer induce the transcription of miR-144 in hepatocytes. Thus, we measured the levels of H₂O₂ secreted in the media of LMs and hepatocytes after silencing miR-144 in LMs. Secretion of H₂O₂ was significantly reduced in both LMs and hepatocytes (FIG. 4E-F), suggesting that silencing of miR-144 in LMs could alleviate oxidative stress in the liver microenvironment. We then measured the levels and phosphorylation of GATA4 in hepatocytes following the silencing of miR-144 in LMs. GATA4 phosphorylation was reduced in hepatocytes upon treatment with GeRP-amiR-144 (FIG. 4G), corroborating the notion that silencing miR-144 in LMs leads to a reduction in miR-144 transcription in hepatocytes.

In order to further investigate the regulation of miR-144 and ROS secretion, we exposed 286 human Non-Parenchymal Cells (NPCs) to H₂O₂ and silenced miR-144 (amiR-144) in vitro. With H₂O₂ treatment, we observed increased expression of GATA4, leading to increased miR-144 expression which was significantly blunted by amiR-144 (FIG. 4H-I). As expected, H₂O₂ significantly increased NRF2 antioxidant target gene expression which was further enhanced with silencing of miR-144 (FIG. 4J). Following the H₂O₂ treatment, human NPCs secreted significantly more H₂O₂ which was mitigated by amiR-144 (FIG. 4K). This suggests a feedback loop between extracellular ROS, intracellular ROS and miR-144/GATA4 expression.

Using a 3D-culture model of human primary hepatocytes (liver spheroids) (16) we found that treatment with extracellular H₂O₂ was sufficient to significantly induce miR-144 expression (FIG. 4L). To more closely mimic the in vivo liver environment, we added NPCs to the liver spheroids and treated them with free fatty acids (FFAs) to recapitulate the lipid overload in obese livers. Three types of liver spheroids were formed: (i) with normal miR-144 levels in both hepatocytes and NPCs, (ii) miR-144 silenced only in NPCs or (iii) only in hepatocytes (FIG. 4M). As expected, treatment with FFAs boosted miR-144 expression in liver spheroids (FIG. 4N). Silencing of miR-144 in the NPCs alone reduced FFAs-driven miR-144 induction, as seen in the mouse model (FIG. 4N). Treatment of hepatocytes with the antagomiR-144 also decreased miR-144 in spheroids treated with FFAs (FIG. 4N). Furthermore, miR-144 silencing in either in the NPCs or hepatocytes, resulted in an increase in expression of the NRF2 antioxidant target genes (FIG. 4O). These results highlighted the importance of miR-144 expressed by LMs and hepatocytes in the regulation of the endogenous antioxidant response. Considering the low percentage of LMs in liver (6-10%) (8), these results support the importance of LMs in the regulation of miR-144 expression and ROS secretion in the liver during obesity in mice and humans.

Silencing miR-144 in LMs Reduces Oxidative Stress and Improves Hepatic Metabolism in Insulin Resistance

Silencing miR-144 specifically in LMs reduced the expression of miR-144 in hepatocytes via reduction of GATA4 phosphorylation. Since GATA4 phosphorylation is triggered by oxidative stress we hypothesized that miR-144 silencing in LMs could reduce oxidative stress in the obese liver. We first measured levels of NRF2 protein and ROS and RNS in livers of obese mice treated with either GeRP-amiR-144 or GeRP-Scr. We observed a significant increase of NRF2 protein levels in livers of mice treated with GeRP-amiR-144 in the whole liver, in LMs and in hepatocytes (FIG. 5A-C), followed by increased expression of NRF2 target genes (Nqo1, Gstp1 and Ces2G) (FIG. 5D-E). Consistently silencing miR in LMs reduced ROS and, to a lesser extent, RNS in liver of treated mice compared to controls (FIG. 5F-H). This result confirmed the hypothesis that silencing miR-144 in LMs results in reduction of miR-144 levels in hepatocytes due to a decreased production of ROS. To further investigate this mechanism, we silenced miR-144 in lean healthy mice that produce physiological levels of ROS. Following treatment with Fluorescein (FITC) labeled-GeRPs, LMs containing GeRPs (CD45+/F4/80+/Cd11b+/FITC+), empty LMs (CD45+/F4/80+/Cd11b+/FITC−) and empty non-LMs Non-Parenchymal Cells (NPCs) (CD45−/FITC−) were sorted by flow cytometry, while hepatocytes were isolated as described in methods section. amiR-144 treatment did not influence percentage of resident and recruited macrophages (FIG. 51). Moreover, while miR-144 was successfully silenced in FITC+ LMs, we observed no effect on the levels of miR-144 in any other cell fractions (FIG. 5J-M). These data further confirmed that the reduction in miR-144 levels in hepatocytes following silencing LMs was due to a decreased oxidative stress leading to a diminished transcription of miR-144 via GATA4.

We then assessed whether the increase in NRF2 and reduced liver oxidative stress had an effect on whole body metabolism. We did not observe any significant changes in body weight or total TG content in the liver upon treatment with GeRP-amiR-144 (FIG. 10 (S4) A-C). However, transmission electronic microscopy (TEM) revealed an increase in the number of mitochondria following miR-144 silencing, suggesting an adaptive mechanism to protect hepatocytes against oxidative stress (FIG. 5N-O). Interestingly, levels of stored intracellular glycogen in the liver were increased in mice treated with GeRP-amiR-144 (FIG. 5P). We thus assessed whether silencing miR-144 could impact whole body glucose metabolism. Consistent with the increased glycogen stores, glucose tolerance tests in mice treated with GeRP-amiR-144 showed an improved glucose homeostasis compared to control mice (FIG. 5Q). This effect was specific for miR-144 as we did not detect any differences following treatment with GeRP-amiR-192 (FIG. 10 (S4) D). These data suggested that miR144, expressed by LMs and hepatocytes, could contribute to liver oxidative stress and glucose homeostasis in obesity. All together these results demonstrated that miR-144 is able to decrease the levels of NRF2, resulting in an impaired antioxidant response in the livers of obese insulin resistant mice and humans.

### Discussion

In this study we investigated the role of LMs in the regulation of the antioxidant response in the livers of obese insulin resistant humans and mice (FIG. 6). We first confirmed the oxidative stress triggered by obesity in murine and human livers. Previous studies suggested that oxidative stress and associated damage could represent a link between obesity and liver disease (17, 18, 19, 20).

The contribution of LMs to oxidative stress in the liver has been debated and despite several reports of LMs activation leading to unbalanced and detrimental ROS production in liver diseases (21), the direct role of LMs in the regulation of oxidative stress during the initial disease state is still unknown. Indeed, studies described macrophages and particularly LMs as the major source of ROS (22), primarily referred to pro-inflammatory activated macrophages (23). However, we have recently demonstrated that LMs do not undergo a pro-inflammatory activation with obesity or insulin resistance in mice and humans (8). Herein we showed that lipid oxidation and the anti-oxidative response were among the most significantly impaired pathways in the two models of obesity.

The main mechanism protecting against oxidative stress is the NRF2/ARE pathway, which induces the expression of antioxidant response genes (24). We found that NRF2 protein levels were dramatically reduced in obese mice and human individuals, suggesting an impaired antioxidant response. KEAP1 has been extensively described as the main regulator of NRF2 at the post-transcriptional level. In the absence of oxidative stress, the interaction between NRF2 with KEAP1 facilitates the proteasomal degradation and rapid turnover of NRF2 (25, 26). Conversely, under conditions of oxidative stress the modification of KEAP1 cysteine residues leads to a change in its conformation that releases NRF2, which then translocates to the nucleus where it binds to the ARE, subsequently activating the transcription of antioxidant genes (27). Inflammatory activation of macrophages has been associated with a higher production of itaconate from citrate in the TCA cycle which could then activate NRF2 through alkylation of KEAP1 (28). In that context, itaconate was described as an anti-inflammatory metabolite able to reduce oxidative stress.

In this study we found that Nrf2 mRNA expression remains unchanged upon oxidative stress induced by obesity. Furthermore, levels of KEAP1 and ubiquitination of NRF2 did not change during obesity, suggesting a different post-transcriptional mechanism regulating NRF2 protein levels independently of KEAP1. Considering that LMs do not undergo an inflammatory activation during obesity, the different NRF2 regulation could be depend from the type and kinetics of stimulus. In the study by Mills et al, potent and acute inflammatory stimuli were used (lipopolysaccharide or IFN-β), while in our study macrophages were exposed to chronic lipid overload resulting in an oxidative stress that did not induce an inflammatory activation and probably requires a more sustainable mechanism of regulation than rapid degradation. We also do not exclude a differential mechanism of regulation due to the use of different macrophage cell types (blood and bone-marrow derived macrophages versus liver macrophages).

miRNAs are short, single-stranded non-coding RNAs of approximately 21-23 nucleotides in length (29) which bind to target mRNAs at the 3′UTR region and exerts their function through mRNA degradation or protein translation inhibition (30). We hypothesized that NRF2 could be targeted by a miRNA and thus we analyzed the miRNome of LMs from obese and healthy mice. Among the upregulated miRNAs detected in obese LMs, miR-144 had previously been reported to reduce NRF2 protein levels in cancer (31). Interestingly, miR-144 levels were also highly increased in whole livers of obese mice and humans. More importantly, insulin resistance was associated with a dramatic increase in miR-144 in humans.

In order to study the mechanism whereby miR-144 was regulated by insulin resistance we first performed in silico predictive analysis which detected binding sites for the transcription factor GATA4 nearby the TSS of miR-144. ChIP analysis revealed that GATA4 indeed bound the promoter region of miR-144, subsequently inducing its transcription. This was consistent with reports that miR-144 transcription is regulated by the transcription factor GATA4 in cardiomyocytes (32). In mice, GATA4 activation via ERK-mediated phosphorylation in cardiomyocytes has been previously shown to be induced by hyperglycemia (14). Our investigations corroborated these findings as we observed an increased ERK phosphorylation in obese patients compared to lean controls. In addition, GATA4 phosphorylation was dramatically reduced in livers of obese Erk1−/− mice and consequently miR-144 levels remained unchanged upon obesity. Importantly, while the levels of miR-144 were increased in OIR compared to OIS subjects, levels of ERK1/2 phosphorylation were comparable. However, levels of GATA-4 protein were higher in OIR, suggesting that the difference in miR-144 between OIR and OIS individuals might not only be due to the activation of GATA4 but also by its protein levels.

Taking advantage of the GeRP technology to specifically manipulate gene expression in LMs we observed a decrease in miR-144 levels in LMs and miR-144 levels were also reduced in hepatocytes. This latter result was surprising since GeRPs cannot be delivered to non-phagocytic cells such as hepatocytes (33, 8, 34). The specific bio-distribution of GeRPs was confirmed by targeting another miRNA, miR-192, which was only silenced in LMs and not in hepatocytes. Based on these findings we first hypothesized that LMs could deliver miR-144 to hepatocytes through EV, and silencing miR-144 in LMs could therefore result in decreased miR-144 in both LMs and hepatocytes. However, EV produced by LMs did not contain miR-144, which remained undetectable following silencing of miR-144.

The other possible explanation was that silencing miR-144 in LMs could reduce ROS production in the liver and consequently decrease the expression of miR-144 in hepatocytes. Consistent with this hypothesis the knockdown of miR-144 in LMs significantly diminished oxidative stress markers in whole livers of obese mice, suggesting a crosstalk between LMs and hepatocytes. This hypothesis was confirmed by our findings revealing ROS release by both LMs and hepatocytes was decreased following miR-144 specific silencing in LMs. However, we found that hepatocytes may also play a role in the regulation of miR-144. Indeed, silencing miR-144 in human primary hepatocyte 3D-cultures (16) exposed to H₂O₂ or FFA was able to trigger the antioxidant response efficiently. ROS could therefore act a secondary messenger that contribute to a vicious cycle whereby LMs communicate with hepatocytes in order to increase miR-144 expression leading to an impaired antioxidant response. Interestingly ROS release has been mainly described in pro-inflammatory macrophages (26, 35, 36, 37), while we found that ROS production could be dissociated from inflammation in LMs in obesity. In addition, GATA4 phosphorylation was reduced in hepatocytes upon silencing of the miR-144 in LMs, thus confirming a major role of LMs in the regulation of miR-144 transcription induced by oxidative stress. Since NRF2 protein levels were increased, the observed reduction in oxidative stress upon miR-144 silencing in LMs could be explained by a restored endogenous antioxidant response. Although additional work would be needed to study the mechanism whereby NRF2 restoration drives the antioxidant response at the subcellular level, the increased number of mitochondria in the livers of mice treated with the antagomiR-144 suggest an effect on mitochondrial biogenesis, as previously described (38, 39).

Finally, silencing miR-144 expression in LMs significantly improved glucose tolerance and increased liver glycogen stores in obese mice. Consistent with a role of ERK1/2 in the activation of GATA4 and subsequent increase in miR-144, ob/ob-Erk1−/− mice had a similar phenotype than silencing miR-144 upon obesity (15). While the molecular mechanism whereby Erk1 deficiency improved metabolism in ob/ob mice is not fully elucidated, our findings suggest that a better antioxidant response might explain the improved liver function in these mice. Indeed, we demonstrated that silencing miR-144 in human NPCs previously treated with H₂O₂ boosted the endogenous antioxidant response and influenced GATA4 expression, suggesting a feedback loop between GATA4 and miR-144 transcription levels in response to ROS.

While several studies have highlighted the beneficial effects of NRF2 activation (40, 41), long-term NRF2 stimulation has been associated with liver fibrosis (42), reductive stress (43) and promotion of pre-existing malignancies (44). The major advantage of the GeRP technology is based on its ability to manipulate miRNA expression in a transient and specific manner in LMs, while leaving other cells and macrophages in the body unaffected. This is particularly important since attempts to reduce oxidative stress using exogenous antioxidants such as vitamin C, vitamin E or β-carotene had no beneficial or even deleterious effects (45, 46, 47). The lack of efficacy of these exogenous antioxidants is thought to be due to both non-specific systemic effects and a decrease in the endogenous antioxidant response. This highlights the importance of a targeted approach to increase the endogenous antioxidant response in order to reduce oxidative stress.

In summary, this study unveils the pivotal role of LMs in the regulation of systemic metabolism in obesity-induced insulin resistance in mice and humans. Specifically, LMs produce miRNAs able to impair the antioxidant capacity of the liver in response to excessive lipid accumulation as observed in obese insulin resistance. We cannot exclude the importance of hepatocytes in the regulation of miR-144 expression, and the antioxidant response in obesity. However, despite the low percentage of LMs in liver, we observed a dramatic global effect of mir-144 specific silencing in LMs. Therefore, specific targeting of LMs in order to attenuate the burden of oxidative stress during obesity through re-activation of the endogenous antioxidant response could represent a novel therapeutic approach for metabolic diseases.

Materials and Methods

Human Subjects

Liver samples were obtained from a total of 15 individuals, including ten obese patients (body mass index (BMI) between 35 and 42 kg/m2), undergoing laparoscopic Roux-en-Y gastric bypass surgery at Danderyd hospital or Ersta hospital in Stockholm. Liver cells from five non-obese patients were obtained from liver donors and isolated by the Liver Cell Laboratory at the Unit of Transplantation surgery, Department of Clinical Science, Intervention and Technology (CLINTEC) at Karolinska Institutet. None of the participants had any previous history of cardiovascular disease, diabetes, gastrointestinal disease, systemic illness, alcohol abuse, coagulopathy, chronic inflammatory disease, any clinical sign of liver damage or surgical intervention within six months prior to the study. Patients did not follow any special diet before the surgery. Insulin sensitivity was assessed by homeostatic model assessment (HOMA-IR). Of the obese patients, five patients with Homa-IR<2 were defined as obese insulin sensitive (OIS) and five with Homa-IR<4 as insulin resistant (01R). Hepatic steatosis index (HIS) was calculated as in (48). The Regional Ethical Committee in Stockholm approved the study and all the subjects gave written informed consent for all procedures prior to their participation. Liver cells from nonobese patients were obtained from liver donors and isolated by the Liver Cell Laboratory at the Unit of Transplantation surgery, Department of Clinical Science, Intervention and Technology (CLINTEC) at Karolinska Institutet.

Mice and Diet

Four-week-old wild-type C57BL/6J (WT) and five-week-old ob/ob males were obtained from Charles River Laboratories International, Inc. and maintained on a 12-hour light/dark cycle. Animals were given free access to food and water. C57BL/6J WT mice were fed a high-fat diet (HFD) composed of 60% calories from fat, 20% from carbohydrates, 20% from proteins (Research Diets Inc.; D12492) at five weeks of age. Control mice were fed a normal chow diet. All procedures were performed in accordance with guidelines approved by the Swedish Ethical Committee in Stockholm (Stockholms södra djurförsöksetiska nämnd).

GeRPs Administration by i.v. Injection In Vivo

GeRPs were prepared as previously described (12). WT mice fed a HFD for 8 weeks were first randomized according to their body weight and glucose tolerance. Then mice were treated with 12.5 mg/kg GeRPs loaded with miRIDIAN microRNA mmu-miR-144-5p hairpin inhibitor (GeRP-miR-144) (Dharmacon; IH-311182-01-0005) or with miRIDIAN microRNA Hairpin Inhibitor Negative Control #1 (Dharmacon; IN-001005-01-05) (247 μg/kg) and Endoporter (2.27 mg/kg) (scr). Mice received six doses of fluorescently labeled GeRPs by i.v injections over 15 days.

Isolation of LMs and Hepatocytes from Mice

LMs and hepatocytes were isolated as previously described (49). Briefly, livers of anesthetized mice were first perfused with calcium-free Hank′ balanced salt solution (HBSS), followed by collagenase digestion. After digestion the hepatocytes were released by mechanical dissociation of the lobes and underwent several steps of filtration with 94 calcium-containing HBSS and centrifugation at 50 g for 3 min. The resulting hepatocyte pellet was washed twice and plated. The supernatant containing non-parenchymal cells was loaded on a Percoll gradient (25% and 50%) and centrifuged for 30 min at 2300 rpm and 4° C. The interphase ring with enriched LMs was collected. The cells were then plated for 30 min and washed twice before RNA or proteins were extracted for subsequent analyses.

Isolation of NPCs from Humans

Freshly obtained liver biopsies were cut into small pieces and immediately digested in RPMI media containing collagenase II (0.25 mg/ml, Sigma C6885) and DNase I (0.2 mg/ml, Roche 1010415900) at 37° C. for 30 min. Single cell suspensions were filtered through a cell strainer (75 μm) and centrifuged at 50 g for 3 minutes. The supernatant containing NPCs were loaded on a Percoll gradient and LMs isolated as described above.

H₂O₂ Treatment

Human NPCs were treated with 500 μM H₂O₂ for 30 minutes (followed by maintenance in low glucose/insulin medium for 20 hours) and treated with scr or GeRP-amiR-144 as described above. Cells were harvested after 24 hours and downstream experiments were performed as listed below.

Metabolic Analyses in Mice

Glucose tolerance test (IP-GTT) was performed on the day of the last GeRP injection and following a 6 hr fast. A dose of 1 g/kg glucose was injected i.p and blood glucose levels were measured using a glucometer at defined time points from the tail vein. The following day mice were sacrificed, and tissues were collected for subsequent analyses.

Isolation of RNA, microRNA, Real-Time Quantitative PCR and RNA Library Preparation

Total RNA and microRNAs extraction and purification was performed using the TRIzol Reagent or the (Thermo Fisher Scientific-15596018) or the miRNeasy mini kit (Qiagen; 217004) following the manufacturers' protocol. For miRNA analyzes 100 ng total RNA were reverse-transcribed and amplified in real-time PCR using miScript-System including miScript RT-Kit (Qiagen; 218161), miScript SYBR-Green PCR-Kit and miScript Primer Assay miRBase v12 (Qiagen; 2i8076) according to the manufacturer's protocol. Specific primers for hsa-miR-144 (Qiagen; 218300), mmu-miR-144 (Qiagen; MS00024213), mmu-miR-532 (Qiagen; MS00002611) and mmu-miR-192 (Qiagen; MS00011354) were used for stem-loop qPCR. For internal control the expression of the small nuclear RNAs RNU6B (Qiagen; MS00033740) was determined. For real time qPCR, cDNA was synthesized from 0.5 μg of total RNA using iScript cDNA Synthesis Kit (Bio-Rad, Hercules, Calif., USA) according to the manufacturer's instructions. Synthesized cDNA forward and reverse primers along with the Sso Advanced Universal SYBR Green Supermix were run on the CFX96 Real-time PCR System (Bio-Rad, Hercules, Calif., USA). 60S acidic ribosomal protein PO (rplp0) or b-actin were used as reference genes in mice and humans. Primer sequences used for qPCR:

mouse Nrf2
(SEQ ID NO: 12)
FW: 5′-CGAGATATACGCAGGAGAGGTAAGA-3′;
(SEQ ID NO: 13)
REV: 5′-GCTCGACAATGTTCTCCAGCTT-3′,
mouse Gata4
(SEQ ID NO: 14)
FW: 5′-CCCTACCCAGCCTACATGG-3′;
(SEQ ID NO: 15)
REV: 5′-ACATATCGAGATTGGGGTGTCT-3′,
mouse Nqo1
(SEQ ID NO: 16)
FW: 5′-TTCTGTGGCTTCCAGGTCTT-3′;
(SEQ ID NO: 17)
REV: 5′-AGGCTGCTTGGAGCAAAATA-3′,
mouse Gstp1
(SEQ ID NO: 18)
FW: 5′-TGTCACCCTCATCTACACCAAC-3′;
(SEQ ID NO: 19)
REV: 5′-CAGGGTCTCAAAAGGCTTCAG-3′,
mouse Ces2G
(SEQ ID NO: 20)
FW: 5′-TCTCTGAGGTGGTTTACCAAACG-3′;
(SEQ ID NO: 21)
REV: 5′-CCTCTCAGACAGCGCACCAG-3′,
mouse β-actin
(SEQ ID NO: 22)
FW: 5′-TCTACAATGAGCTGCGTGTGG-3;
(SEQ ID NO: 23)
REV: 5′-GTACATGGCTGGGGTGTTGAA-3′,
human NRF2
(SEQ ID NO: 24)
FW: 5′-CAGCGACGGAAAGAGTATGA-3′;
(SEQ ID NO: 25)
REV: 5′-TGGGCAACCTGGGAGTAG-3′,
human NQO1
(SEQ ID NO: 26)
FW: 5′-GGCAGAAGAGCACTGATCGTA-3′;
(SEQ ID NO: 27)
REV: 5′-TGATGGGATTGAAGTTCATGGC-3′,
human GSTP1
(SEQ ID NO: 28)
FW: 5′-GTAGTTTGCCCAAGGTCAAG-3′;
(SEQ ID NO: 29)
REV: 5′-AGCCACCTGAGGGGTAAG-3′,
human CES2G
(SEQ ID NO: 30)
FW: 5′-TCTTCGCTTGTTGTGTCC-3′;
(SEQ ID NO: 31)
REV: 5′-CGAAGGAGAAAGGCAATGAC-3′,
human GATA4
(SEQ ID NO: 32)
FW: 5′-TTCCAGCAACTCCAGCAACG-3′;
(SEQ ID NO: 25)
REV: 5′-GCTGCTGTGCCCGTAGTGAG-3′,
human RPLP0
(SEQ ID NO: 33)
FW: 5′-CAGATTGGCTACCCAACTGTT-3′;
(SEQ ID NO: 34)
REV: 5′-GGGAAGGTGTAATCCGTCTCC-3′.
For ChIP-qPCR the following hChIPmiR144/451
promoter primers were used:
(SEQ ID NO: 35)
FW: 5′-CCTGGGCTGTGCCTGACCAC-3′;
(SEQ ID NO: 36)
REV: 5′-AGCACTGTGAGGGGCTGGGG-3′.

For library preparation, RNA integrity was determined using an Agilent Bioanalyzer. Libraries from mouse RNA were prepared using TruSeq Stranded mRNA kit (Illumina; RS-122-2201). Libraries for small-RNA sequencing were prepared using TruSeq Small RNA kit (Illumina; RS-930-1012) The concentration of indexed libraries was quantified by RT-qPCR using the Universal Kapa Library Quantification Kit (KAPA Biosystems). Final libraries were normalized and sequenced on an Illumina HiSeq 3000 sequencer.

Liver Spheroids

antagomiR Transfections

Cryopreserved primary human hepatocytes (Heps) (Bioreclamation IVT, USA) were mixed with a pre-incubated mixture of Lipofectamine RNAiMAX (Invitrogen; 13778030) and amiR/inhibitor constructs (1 nmol amiR/inhibitor per 300,000 cells) in OptiMEM (Gibco; 31985). In the case of co-culture transfections, cryopreserved hepatocytes and isogenic non-parenchymal cells (NPCs) (Bioreclamation IVT, USA) were transfected separately in suspension with a pre-incubated mixture of Lipofectamine RNAiMAX and amiR/inhibitor constructs (1 nmol amiR/inhibitor per 300,000 cells) in OptiMEM. Cells were transfected for 5 hours with occasional agitation of the suspension. All transfections were performed using low glucose/insulin medium (PAN-Biotech, Germany; P04-29050 supplemented with 5.5 mM D-glucose, 0.1 nM insulin, 2 mM L-glutamine, 100 units/mL penicillin, 100 μg/ml streptomycin, 5.5 μg/ml transferrin, 6.7 ng/ml sodium selenite, 100 nM dexamethasone, and 10% FBS).

Spheroid Formation

Spheroids were formed from hepatocytes alone or from co-cultures of hepatocytes and NPCs as indicated. In the case of co-cultures, separately-transfected hepatocytes and NPCs were seeded at a ratio of 3:1 (Heps:NPCs). Cells were seeded in ultra-low attachment 96-well plates (Corning; CLS3471) as previously described (16) and were cultured in low glucose/insulin medium. Plates were centrifuged at 180×g for 2 min. Plates were centrifuged again if cells were not well aggregated. After 6 days, when the spheroids were sufficiently compact, 50% of the medium was exchanged for serum-free medium.

Free Fatty Acid Supplementation

Free fatty acids were conjugated to 10% bovine serum albumin (Sigma-Aldrich) at a molar ratio of 1:5 for 2 hours at 40° C. Spheroids were treated with 240 μM oleic acid (Sigma-Aldrich) and 240 μM palmitic acid (Sigma-Aldrich) in high glucose/insulin medium (Gibco; 11965092 supplemented with 11.1 mM D-glucose, 1.7 μM insulin, 2 mM L-glutamine, 100 units/mL penicillin, 100 μg/ml streptomycin, 5.5 μg/ml transferrin, 6.7 ng/ml sodium selenite, 100 nM dexamethasone, and 10% FBS) for 5 days. Untreated spheroids were maintained in low glucose/insulin medium. All treatments were performed 8 days after spheroid seeding.

H₂O₂ Treatment

Spheroids were treated with 500 μM H₂O₂ for 30 minutes (followed by maintenance in low glucose/insulin medium for 20 hours). All treatments were performed 8 days after spheroid seeding and in low glucose/insulin medium.

Extracellular Vesicle Isolation

LMs were isolated as described above and cultured in RPMI (Sigma Aldrich; R0883) medium with 10% EV-depleted FBS (ThermoFisher Scientific; A25904DG). Extracellular vesicles (EV) were isolated as previously described (50). Briefly, cell culture media was centrifuged for 10 mins at 300 g to pellet cellular debris. Supernatants were combined with phosphate buffered saline (PBS) (ThermoFisher Scientific; AM9625), transferred to ultracentrifugation tubes (Polyallomer Quick-Seal ultra-clear 16 mm×76 mm tubes, Beckman Coulter) and centrifuged at 120,000×g for 2 hours to pellet extracellular vesicles. Isolated extracellular vesicles were resuspended in 100 μL PBS and utilized for extracellular vesicles characterization as detailed below.

Extracellular Vesicles microRNA Analysis

Total RNA and microRNAs isolation and stem loop qPCR were performed on isolated EV as described above. Specific primers for RNU6B, mmu-miR-126-3p and mmu-miR-144 (QIAGEN) were used for qPCR.

Extracellular Vesicle Size, Concertation and Zeta Potential

Extracellular vesicles size and concertation was determined by dynamic light scattering using the ZetaView (Particle Metrix, Germany) platform.

Nuclei and Library Preparation for GRO-Seq

GRO-seq was performed as previously described (51), with minor modifications for mouse liver macrophages samples. Nuclei were extracted from liver macrophages (3-4 pooled mice/group) using hypotonic buffer, and visually inspected for quality under a microscope with DAPI staining. The total number of nuclei was determined using a Countess Automated Cell Counter (Bio-Rad). Nuclear run-on was performed using Br-UTP followed by enrichment with anti-Br-UTP antibodies, reverse transcription and library preparation.

Western Blot, Immunoprecipitation and Chromatin Immunoprecipitation Assay

30 μg of proteins were fractionated by SDS-polyacrylamide gel electrophoresis using precast 4-12% gradient gels (ThermoFischer Scientific; NP0321BOX), transferred to polyvinylidene difluoride membranes (ThermoFischer Scientific; LC2005) and probed with a 1:1000 dilution of the primary antibodies indicated below. This was followed by incubation with the appropriate HRP-conjugated secondary antibody (Abcam; ab6721 or ab6789). Finally, bound secondary antibodies were visualized by ECL detection reagent (BioRad; 1705060) and images were acquired by an imaging system equipped with CCD camera (ChemiDoc, Bio-Rad). Immunoprecipitations for KEAP1 (Santa Cruz Biotechnology, sc-514914) and NRF2 (Abcam; ab137550) were performed on 1000 μg of proteins. Lysates were incubated overnight with Agarose Protein G plus mixture (Pierce; 22851) and protein complexes were eluted in Laemmli buffer. Then, Western Blot analysis were performed as described above. Chromatin immunoprecipitation was performed using EpiQuik Tissue Chromatin Immunoprecipitation (ChIP) Kit (EpiGentek; P-2003) according the manufacturer's instructions. For ChIP, samples were incubated overnight with GATA-4 monoclonal antibody (ThermoFisher; MA5-15532) and Anti-Histone H3 (tri methyl K4) antibody-ChIP grade (Abcam; ab8580). The following primary antibodies were used: NRF2 (Abcam; ab137550), KEAP1 (Santa Cruz Biotechnology; sc-514914), ubiquitin (Abcam; ab7780); p44/42 MAPK (Erk 1/2) (Cell Signaling; 4965S), Phospho p44/42 MAPK (Erk1/2) (Thr202/Tyr204) (Cell Signaling; 4370S), GATA4 (phospho S105) antibody (Abcam; ab92585), GATA4 antibody (Abcam; ab227512), and b-actin (Abcam; ab179467). Quantification of the signal was assessed using ImageJ software.

Histology

Paraffin-embedded tissue sections of pancreas were used for hematoxylin-eosin staining and frozen sections of the liver for Oil Red O staining. The slides were scanned with Panoramic 250 Slide Scanner.

Mouse Biochemical Parameters

Total triglycerides content was determined using colorimetric techniques using commercially available reagents (Roche; TG 12016648).

Malondialdehyde and Reactive Oxygen Species Content Measurement

Malondialdehyde content was measured using a Lipid Peroxidation (MDA) Assay Kit (Colorimetric/Fluorometric) (Abcam; ab118970). Reactive Oxygen Species were determined using OxiSelect™ In Vitro ROS/RNS Assay Kit (Green Fluorescence) (NordicBiosite; STA-347) according to manufacturer's instructions. Intracellular ROS were determined using DCFDA/H2DCFDA—Cellular ROS Assay Kit (Abcam; ab113851). Intracellular RNS levels were assessed using Cell Meter™ Fluorimetric Intracellular Nitric Oxide (NO) Activity Assay Kit *Orange Fluorescence Optimized for Microplate Reader (AAT Bioquest; 16350). Extracellular release of H₂O₂ was measured using Amplex™ Red Hydrogen Peroxide/Peroxidase Assay Kit (Life Technologies; A22188). All assays were performed following manufacturer's instructions.

Transmission Electron Microscopy (TEM)

Preparation for transmission electron microscopy (TEM) was performed according to a published protocol (52). For numerical density measurements of mitochondria in the liver, digital images of liver cell cytoplasm were randomly taken at a final magnification of 5000×. Printed digital images were used and the number of mitochondria was calculated by point counting using ImageJ software. 20 sections/mouse were analysed.

Flow Cytometry

Non-parenchymal cells were stained with the following fluorophore-conjugated primary antibodies and dyes: Viability Dye SYTOX Blue (ThermoFisher Scientific, S34857); F4/80-APC (BioRad, CI:A3-1; MCA497APC), CD11b-PE-Cy7 (BD Biosciences, MI/70; 561098), CD45-PECF594 (BD Biosciences, 30-F11; 562420). Cells were washed two times with FACS buffer (1% BSA in PBS) after staining, and samples were sorted using a BD FACSAria Fusion.

Bioinformatics

Retrieving Raw Sequencing Data

Signal intensities were converted to individual base calls during the sequence run using the system's real time analysis (RTA) software. Sample de-multiplexing and conversion to fastq-files was performed using Illumina's bcl2fastq software with all default options. The distribution of reads per sample in a lane was within reasonable tolerance.

mRNA-Seq Alignment and Gene Quantification for HFD and ND Mice

Raw fastq-files (PRJNA483744) (8) were aligned against the murine genome version mm10 using TopHat version v2.0.13 (53) with all default options. BRM files containing the alignment results were sorted according to the mapping position. mRNA quantification was performed using FeatureCounts from the Subread package (54) against the GRCm38-gencode transcripts database version seven (gencode.vM7.annotation.gtf) and the GRCh38-genocode transcripts database version 24 (gencode.v24.annotation.gtf) to obtain read counts for each individual Ensembl gene.

GRO-Seq Data Processing and Gene Quantification for HFD and ND Mice

Raw fastq-files (PRJNA483744) (8) were aligned against the murine genome version mm10 using BWA (51) with samse option. Uniquely mapped reads were extended to 150 bp in the 5′ to 3′direction and used for downstream analysis. Nascent transcription of genes was measured using GRO-seq reads mapped to the sense strand of the gene in a 10 kb window (+2 kb to +12 kb relative to transcription start site (TSS)) within the gene symbol annotated gene body. Smaller genes between 2 kb and 12 kb in length were quantified using smaller window size, from +2 kb to the transcription end site (TES). For genes shorter than 2 kb, the entire gene body was used for the quantification. The mapped reads within each gene quantification window were counted using bedtools with the intersect option (55) and expressed as reads per kb per million reads (RPKM). Genes with transcription levels greater than 0.3 RPKM were considered as being actively transcribed. Genes that were not transcribed throughout all conditions were eliminated before downstream analysis. A gene was defined as ‘differential’ between a given pair of conditions if it was transcribed in either condition and the fold-change was greater than 1.5 (either up or down).

Analysis of RNA Sequencing Data from Ob/Ob and Wt Mice

Raw reads were aligned to the mouse genome mm10 (genome build GRCm38.p5) using STAR aligner (56) and followed by expression quantification at gene level based on Gencode M14 annotation using the Cufflinks pipeline (57). Cuffdiff (58) was used to identify genes differentially expressed between ob/ob and wt mice. GO enrichment and pathway over-representation analysis were further performed on differentially expressed genes between conditions (adjusted p-value <0.05 and log 2-scale fold change >1 or <−1). Raw fastq files and processed data are available in GEO repository (GSE132801, GSE132800).

NRF2 Targets

NRF2 target genes (antioxidant, phase 1 and phase 2) were downloaded from the WikiPathways (Pathway: WP2884) (59). Human gene names were converted into mouse orthologues for downstream analysis using Ensembl BioMart version 92.

Analysis of Small RNA Sequencing Data

After removing the adaptors from the raw reads by Cutadapt (60), ShortStack (61) was used to align the small RNA reads against GENCODE mouse primary assembly (release M14, GRCm38.p5) and further identify the miRNA clusters in de-novo mode. ShortStack quantified the expression of the most abundant RNAs (MajorRNA) at locus as reads per million (RPM) but ignored the quantification for less abundant RNAs (MinorRNA) at the same locus. This could result in false negative discovery of certain miRNAs which are truly expressed in the samples but show no expression due to the quantification. Here, post-processing was performed to quantify the less abundant RNAs by retrieving read counts from the MinorRNA alignments and converted into RPMs. All quantified miRNAs were then annotated into miRBase by aligning the sequences against miRBase mature miRNA sequence database using BLAST (62). All the miRNAs quantified from each sample were then pulled together into an expression matrix for downstream analysis. ANOVA was used to identify miRNAs differentially expressed between conditions based on adjusted pvalue <0.05 and at least one condition has median RPMs over 2. Raw fastq files and processed data are available in GEO repository (GSE132795).

Statistical Analysis

The data were analysed using GraphPad Prism Software. The statistical significance of differences among groups was analysed using ANOVA or Student t-test whenever appropriate. Data were presented as mean±SEM. p-values <0.05 were considered as statistically significant. Sample size for each experiment has been calculated based on previous data collection and as described in Fundamental of Biostatistics by Bernard Rosner (Brooks/Cole CENGAGE Learning; 7th Edition). For animal experiments, although we have always started every experiment with the same number of animals per group, if any individual animal showed any sign of discomfort or an injection failed, we had to terminate the study for this particular animal in accordance with our ethical permit and rigour of the study.

### REFERENCES

1. C. M. Hales, M. D. Carroll, C. D. Fryar, C. L. Ogden, Prevalence of Obesity Among Adults and Youth: United States, 2015-2016. NCHS Data Brief, 1-8 (2017).
2. S. E. Kahn, R. L. Hull, K. M. Utzschneider, Mechanisms linking obesity to insulin resistance and type 2 diabetes. Nature 444, 840-846 (2006).
3. L. P. Bechmann et al., The interaction of hepatic lipid and glucose metabolism in liver diseases. Journal of Hepatology 56, 952-964 (2012).
4. D. H. Ipsen, J. Lykkesfeldt, P. Tveden-Nyborg, Molecular mechanisms of hepatic lipid accumulation in non-alcoholic fatty liver disease. Cell Mol Life Sci 75, 3313-3327 (2018).
5. Q. Liu, S. Bengmark, S. Qu, The role of hepatic fat accumulation in pathogenesis of non-alcoholic fatty liver disease (NAFLD). Lipids Health Dis 9, 42 (2010).
6. C. C. Bell et al., Transcriptional, Functional, and Mechanistic Comparisons of Stem Cell-Derived Hepatocytes, HepaRG Cells, and Three-Dimensional Human Hepatocyte Spheroids as Predictive In Vitro Systems for Drug-Induced Liver Injury. Drug Metab Dispos 45, 419-429 (2017).
7. J. Jager, M. Aparicio-Vergara, M. Aouadi, Liver innate immune cells and insulin resistance: the multiple facets of Kupffer cells. J Intern Med 280, 209-220 (2016).
8. C. Morgantini. et al., Liver macrophages regulate metabolism through non-inflammatory factors. Nat Metab 1, 445-459 (2019).
9. S. Vomund, A. Schafer, M. J. Parnham, B. Brune, A. von Knethen, Nrf2, the Master Regulator of Anti-Oxidative Responses. Int J Mol Sci 18, (2017).
10. S. Furukawa et al., Increased oxidative stress in obesity and its impact on metabolic syndrome. J Clin Invest 114, 1752-1761 (2004).
11. M. Aouadi et al., Orally delivered siRNA targeting macrophage Map4k4 suppresses systemic inflammation. Nature 458, 1180-1184 (2009).
12. G. J. Tesz et al., Glucan particles for selective delivery of siRNA to phagocytic cells in mice. Biochem J 436, 351-362 (2011).
13. H. Dweep, N. Gretz, miRWalk2.0: a comprehensive atlas of microRNA-target interactions. Nature Methods 12, 697 (2915).
14. P. M. Ku et al., Molecular role of GATA binding protein 4 (GATA-4) in hyperglycemia-induced reduction of cardiac contractility. Cardiovasc Diabetol 10, 57 (2011).
15. J. Jager et al., Deficiency in the extracellular signal-regulated kinase 1 (ERK1) protects leptin-deficient mice from insulin resistance without affecting obesity. Diabetologia 54, 180-189 (2011).
16. C. C. Bell et al., Transcriptional, Functional, and Mechanistic Comparisons of Stem Cell-Derived Hepatocytes, HepaRG Cell, and Three-dimensional Human Hepatocyte Spheroids as Predictive In Vitro Systems or Drug-Induced Liver Injury. Drug Metab Dispos 45, 419-429 (2017).
17. S. M. Shin, J. H. Yang, S. H. Ki, Role of the Nrf2-ARE pathway in liver diseases. Oxid Med Cell Longev 2013, 763257 (2013).
18. R. F. Schwabe, D. A. Brenner, Mechanisms of Liver Injury. I. TNF-alpha-induced liver injury: role of IKK, JNK, and ROS pathways. Am J Physiol Gastrointest Liver Physiol 290, G583-589 (2006).
19. R. Sano, J. C. Reed, ER stress-induced cell death mechanisms. Biochim Biophys Acta 1833, 3460-3470 (2013).
20. Y. Sumida, E. Niki, Y. Naito, T. Yoshikawa, Involvement of free radicals and oxidative stress in NAFLD/NASH. Free Radic Res 47, 869-880 (2013).
21. H. Y. Tan et al., The Reactive Oxygen Species in Macrophage Polarization: Reflecting Its Dual Role in Progression and Treatment of Human Diseases. Oxid Med Cell Longev 2016, U.S. Pat. No. 2,795,090 (2016).
22. O. A. Castaneda, S. C. Lee, C. T. Ho, T. C. Huang, Macrophages in oxidative stress and models to evaluate the antioxidant function of dietary natural compounds. J Food Drug Anal 25, 111-118 (2017).
23. L. Formentini et al., Mitochondrial ROS Production Protects the Intestine from Inflammation through Functional M2 Macrophage Polarization. Cell Rep 19, 1202-1213 (2017).
24. W. Tang, Y. F. Jiang, M. Ponnusamy, M. Diallo, Role of Nrf2 in chronic liver disease. World J Gastroenterol 20, 13079-13087 (2014).
25. K. Itoh et al., Keap1 regulates both cytoplasmic-nuclear shuttling and degradation of Nrf2 in response to electrophiles. Genes Cells 8, 379-391 (2003).
26. M. McMahon, K. Itoh, M. Yamamoto, J. D. Hayes, Keap1-dependent proteasomal degradation of transcription factor Nrf2 contributes to the negative regulation of antioxidant response element-driven gene expression. J Biol Chem 278, 21592-21600 (2003).
27. T. Nguyen, P. J. Sherratt, C. B. Pickett, Regulatory mechanisms controlling gene expression mediated by the antioxidant response element. Annu Rev Pharmacal Toxicol 43, 233-260 (2003).
28. E. L. Mills et al., Itaconate is an anti-inflammatory metabolite that activates Nrf2 via alkylation of KEAP1. Nature 556, 113-117 (2018).
29. D. P. Bartel, MicroRNAs: genomics, biogenesis, mechanism, and function. Cell 116, 281-297 (2004).
30. D. P. Bartel, MicroRNAs: target recognition and regulatory functions. Cell 136, 215-233 (2009).
31. C. Sangokoya, M. J. Telen, J. T. Chi, microRNA miR-144 modulates oxidative stress tolerance and associates with anemia severity in sickle cell disease. Blood 116, 4338-4348 (2010).
32. X. Zhang et al., Synergistic effects of the GATA-4-mediated miR-144/451 cluster in protection against simulated ischemia/reperfusion-induced cardiomyocyte death. J Mol Cell Cardiol 49, 841-850 (2010).
33. T. Jourdan et al., Cannabinoid-1 receptor deletion in podocytes mitigates both glomerular and tubular dysfunction in a mouse model of diabetic nephropathy. Diabetes Obes Metab 20, 698-708 (2018).
34. M. Tencerova et al., Activated Kupffer cells inhibit insulin sensitivity in obese mice. Faseb Journal 29, 2959-2969 (2015).
35. E. McNeill et al., Regulation of iNOS function and cellular redox state by macrophage Gch1 reveals specific requirements for tetrahydrobiopterin in NRF2 activation. Free Radic Biol Med 79, 206-216 (2015).
36. M. Mittal, M. R. Siddiqui, K. Tran, S. P. Reddy, A. B. Malik, Reactive oxygen species in inflammation and tissue injury. Antioxid Redox Signal 20, 1126-1167 (2014).
37. L. J. Hofseth et al., Nitric oxide-induced cellular stress and p53 activation in chronic inflammation. Proc Natl Acad Sci USA 100, 143-148 (2003).
38. J. Strom, B. Xu, X. Tian, Q. M. Chen, Nrf2 protects mitochondrial decay by oxidative stress. FASEB J 30, 66-80 (2016).
39. M. Abdalkader, R. Lampinen, K. M. Kanninen, T. M. Malm, J. R. Liddell, Targeting Nrf2 to Suppress Ferroptosis and Mitochondrial Dysfunction in Neurodegeneration. Front Neurosci 12, 466 (2018).
40. H. Zheng et al., Therapeutic potential of Nrf2 activators in streptozotocin-induced diabetic nephropathy. Diabetes 60, 3055-3066 (2011).
41. M. C. Jaramillo, D. D. Zhang, The emerging role of the Nrf2-Keap1 signaling pathway in cancer. Genes Dev 27, 2179-2191 (2013).
42. H. M. Ni et al., Nrf2 promotes the development of fibrosis and tumorigenesis in mice with defective hepatic autophagy. J Hepatol 61, 617-625 (2014).
43. N. S. Rajasekaran et al., Sustained activation of nuclear erythroid 2-related factor 2/antioxidant response element signaling promotes reductive stress in the human mutant protein aggregation cardiomyopathy in mice. Antioxid Redox Signal 14, 957-971 (2011).
44. H. Wang et al., NRF2 activation by antioxidant antidiabetic agents accelerates tumor metastasis. Sci Transl Med 8, 334ra351 (2016).
45. I. D. Podmore et al., Vitamin C exhibits pro-oxidant properties. Nature 392, 559 (1998).
46. E. R. Miller, 3rd et al., Meta-analysis: high-dosage vitamin E supplementation may increase all-cause mortality. Ann intern Med 142, 37-46 (2005).
47. S. M. Lippman et al., Effect of selenium and vitamin E on risk of prostate cancer and other cancers: the Selenium and Vitamin E Cancer Prevention Trial (SELECT). JAMA 301, 39-51 (2009).
48. J. H. Lee et al., Hepatic steatosis index: a simple screening tool reflecting nonalcoholic fatty liver disease. Dig Liver Dis 42, 503-508 (2010).
49. M. Aparicio-Vergara, M. Tencerova, C. Morgantini, E. Barreby, M. Aouadi, Isolation of Kupffer Cells and Hepatocytes from a Single Mouse Liver. Methods Mol Biol 1639, 161-171 (2017).
50. N. Akbar et al., Endothelium-derived extracellular vesicles promote splenic monocyte mobilization in myocardial infarction. JCI Insight 2, (2017).
51. B. Fang et al., Circadian Enhancers Coordinate Multiple Phases of Rhythmic Gene Transcription In Vivo. Cell 159, 1140-1152 (2014).
52. J. Wijkstrom et al., Renal Morphology, Clinical Findings, and Progression Rate in Mesoamerican Nephropathy. Am J Kidney Dis 69, 626-636 (2017).
53. D. Kim et al., TopHat2: accurate alignment of transcriptomes in the presence of insertions, deletions and gene fusions. Genome Biol 14, R36 (2013).
54. Y. Liao, G. K. Smyth, W. Shi, featureCounts: an efficient general-purpose program for assigning sequence reads to genomic features. Bioinformatics 30, 923-930 (2014).
55. R. Quinlan, I. M. Hall, BEDTools: a flexible suite of utilities for comparing genomic features. Bioinformatics 26, 841-842 (2010).
56. Dobin et al., STAR: ultrafast universal RNA-seq aligner. Bioinformatics 29, 15-21 (2013).
57. C. Trapnell et al., Transcript assembly and quantification by RNA-Seq reveals unannotated transcripts and isoform switching during cell differentiation. Nat Biotechnol 28, 511-515 (2010).
58. C. Trapnell et al., Differential analysis of gene regulation at transcript resolution with RNA-seq. Nat Biotechnol 31, 46-53 (2013).
59. N. Slenter et al., WikiPathways: a multifaceted pathway database bridging metabolomics to other omics research. Nucleic Acids Res 46, D661-D667 (2018).
60. M. Martin. Cutadapt removes adapter sequences from high-throughput sequencing reads. EMBnet.journal 17, (1):10-12 (2011).
61. M. J. Axtell, ShortStack: comprehensive annotation and quantification of small RNA genes. RNA 19, 740-751 (2013).
62. J. Ye, S. McGinnis, T. L. Madden, BLAST: improvements for better sequence analysis. Nucleic Acids Res 34, W6-9 (2006).

### EXAMPLE 2—HUMAN SUBJECTS WITH NASH HAVE INCREASED MIR-144 IN SERUM

Strikingly, we found increased miR-144 circulating levels in serum collected from NASH human individuals if compared to OIS and OIR patients without NASH (FIG. 11A). Consistently, miR-144 upregulation was observed also in livers of NASH mouse models (FIG. 11B), as well as in liver biopsies collected from human patients with NASH at different stages (FIG. 11C).

As further confirmation that miR-144 circulating levels were associated with the progression of NASH disease, we performed a further experiment which analyzed miR-144 expression in serum collected from patients with NASH compared to no NASH individuals.

Circulating miRNA extraction was performed using miRNeasy mini kit (Qiagen) following the manufacturers' protocol. Briefly, 700 μl of QIAzol reagent was added to 200 μl of serum. The sample was mixed in a tube, and 2 μl of 0.5 nM of the “spike-in control” cel-miR-39 (Qiagen) was added to the homogenate followed by the addition of 200 μl of chloroform. The “spike-in control” is an exogenous miRNA (isolated from C. elegans, and having the sequence: UCACCGGGUGUAAAUCAGCUUG) added during miRNA isolation to normalize the amount of the miR of interest, and is a common way to do since not all miRNAs are expressed in serum.

After mixing vigorously, the sample was then centrifuged at 12 000 g for 15 min at 4-8° C. The upper aqueous phase was carefully transferred to a new collection tube, and 1.5 volumes of ethanol were added. The sample was then applied directly to columns and washed. Total RNA was eluted in 30 μl of nuclease-free H₂O. Stem-loop RT-qPCR was performed as previously described in the application. The relative expression level of miR-144 was calculated after normalization to the spiked cel-miR-39.

The results are shown in FIG. 12. The stem-loop RT-qPCR experiments demonstrated the upregulation in circulating levels of miR-144 in serum of human individuals with NASH. These results further indicate that miR-144 circulating levels correlate with the development of NASH, and therefore identify miR-144 as biomarker for the prediction and evaluation of NASH disease.

### EXAMPLE 3—EVALUATION OF THE EFFECTS OF DIFFERENT ANTAGOMIRS ON MIR-144 LEVELS

We evaluated the effects of a specific antagomir to miR-144 (“amiR-144”)—in particular, the amiR-144 sequence SEQ ID NO:10 that is described in this application.

NPCs isolated from human individuals were exposed to free fatty acids (“FFA”) for 24 hours in order to drive miR-144 expression, and then treated with either: amiR-144 (SEQ ID NO:10); the microRNA hsa-miR-144-3p hairpin inhibitor (a commercially-available amiR-144 from Dharmacon; (IH-300612-06); and a “scrambled sequence” control sequence (“scr”).

SEQ ID NO:10 is described above in the application. It has the following sequence and modifications: 5′-mC/ZEN/mU mUmAmC mAmGmU mAmUmA mUmGmA mUmGmA mUmAmU mC/3ZEN/-3′, wherein “m” represents a 2′-O-methyl-modified oligonucleotide, and “ZEN” represents N,N-diethyl-4-(4-nitronaphthalen-1-ylazo)-phenylamine, for improved binding affinity and reduced exonuclease degradation.

Liver Macrophages (“LMs”) were isolated from humans and treated with antagomir in the following way. Freshly obtained liver biopsies were cut into small pieces and immediately digested in RPMI media containing collagenase II (0.25 mg/ml, Sigma) and DNase I (0.2 mg/ml, Roche) at 37° C. for 30 min. Single cell suspensions were filtered through a cell strainer (75 μm) and centrifuged at 50 g for 3 minutes. The supernatant containing NPCs was loaded on a Percoll gradient (25% and 50%) and centrifuged to enrich the LMs. These were then plated in presence of a free fatty acids (FFA) mixture (of 240 μM oleic acid (Sigma-Aldrich) and 240 μM palmitic acid (Sigma-Aldrich)) for 24 hours to mimic obese state. NPCs were then transfected with a mixture of Lipofectamine RNAiMAX (Invitrogen; 13778030) and amiR-144 (Dharmacon) or amiR-144 (SEQ ID NO:10) constructs or scrambled controls (1 nmol amiR/scr per 300,000 cells), in OptiMEM medium (Gibco; 31985).

The results are shown in FIG. 13. qPCR highlighted that amiR-144 (SEQ ID NO:10) and amiR-144 (Dharmacon) are able to significantly decrease miR-144 expression levels at a similar rate, and can be successfully used as an amiR-144 in the modulation of miR-144 expression levels.

1. An agent that inhibits microRNA-144 (miR-144) for use in treating or preventing a liver disease and/or liver condition in which oxidative stress is a contributory factor in a subject.

2. (canceled)

3. A method for treating or preventing a liver disease and/or liver condition in which oxidative stress is a contributory factor in a subject, wherein the method comprises administering an agent that inhibits microRNA-144 (miR-144) to the subject.

4. The method of claim 3, wherein the agent decreases miR-144 expression and/or activity in cells of the liver.

5. The method of claim 3, wherein the agent is delivered to cells of the liver.

6. The method of claim 4, wherein the cells of the liver are phagocytic liver cells, hepatocytes, endothelial cells and/or neutrophils.

7. The method of claim 6, wherein the phagocytic liver cells are liver macrophages.

8. The method of claim 5, wherein delivering the agent to cells of the liver results in decreased miR-144 expression and/or activity in cells of the liver, for example in phagocytes, hepatocytes, endothelial cells and/or neutrophils.

9. The method of claim 3, wherein the oxidative stress is induced by obesity, alcohol, environmental pollutants, and/or drugs, such as anti-inflammatory drugs, anti-analgesic drugs, anti-cancer drugs and/or antidepressants.

10. The method of claim 3, wherein the oxidative stress is oxidative stress in cells of the liver.

11. The method of claim 10, wherein the oxidative stress in the liver is characterised by at least one of:
a) increased lipid peroxidation;
b) Reactive Oxygen Species (ROS) increase and/or accumulation;
c) decreased Nuclear Factor Erythroid 2-Related Factor 2 (NRF2) activity and/or protein levels; and/or
d) increased expression and/or activity of miR-144.

12. The method of claim 3, wherein the liver disease and/or liver condition in which oxidative stress is a contributory factor is selected from the group comprising: non-alcoholic steatohepatitis (NASH), non-alcoholic fatty liver disease (NAFLD), fibrosis, cirrhosis, hepatocellular carcinoma (HCC), and/or liver damage induced by alcohol, environmental pollutants, and/or drugs, such as anti-inflammatory drugs, anti-analgesic drugs, anti-cancer drugs and/or antidepressants.

13. The method of claim 3, wherein miR-144 mediates at least one of the following in cells of the liver:
i. NRF2 activity and/or protein levels;
ii. production of extracellular ROS;
iii. GATA4 phosphorylation and/or activity;
iv. levels of intracellular glycogen; and
v. endogenous antioxidant response.

14. The method of claim 3, wherein decreased expression and/or activity of miR-144 causes at least one of the following in cells of the liver:
i) increased NRF2 activity and/or protein levels;
ii) decreased intracellular ROS and/or decreased release of ROS;
iii) decreased phosphorylation and/or activity of GATA4;
iv) increased levels of intracellular glycogen; and
v) restored and/or increased endogenous antioxidant response.

15. The method of claim 3, wherein the agent is selected from the group comprising: a nucleic acid molecule, and a small molecule.

16. The method of claim 15, wherein the nucleic acid molecule is selected from the group comprising: an antisense oligonucleotide and an inhibitory RNA molecule.

17. The method of claim 16, wherein the antisense oligonucleotide comprises a nucleotide sequence which is complementary to at least part of a nucleotide sequence present in SEQ ID NO: 1, SEQ ID NO:2, SEQ ID NO: 3, and/or SEQ ID NO:4.

18. The method of claim 16, wherein the antisense oligonucleotide comprises a nucleotide sequence which is at least 50% complementary to SEQ ID NO: 1 SEQ ID NO:2, and/or SEQ ID NO: 3.

19. The method of claim 16, wherein the nucleotide sequence which is complementary to at least part of a nucleotide sequence present in a miR-144 sequence is 15, 16, 17, 18, 19, 20, 20, 21, 22, or 23 nucleotides in length.

20. The method of claim 16, wherein the antisense oligonucleotide comprises a nucleotide sequence which is complementary to at least part of a nucleotide sequence present in a mature miR-144 sequence.

21. The method of claim 16, wherein the antisense oligonucleotide comprises a nucleotide sequence which is at least 80%, 85%, 90%, 95% or 100% complementary to SEQ ID NO: 4.

22. The method of claim 16, wherein the inhibitory RNA molecule comprises a double-stranded region, and preferably wherein the double-stranded region comprises a nucleotide sequence that is substantially identical and substantially complementary to at least part of a nucleotide sequence present in SEQ ID NO: 1, SEQ ID NO:2 SEQ ID NO: 3, and/or SEQ ID NO: 4.

23. The method of claim 22, wherein the double-stranded region comprises a nucleotide sequence which is at least 50% complementary to at least part of a nucleotide sequence present in SEQ ID NO: 1, SEQ ID NO:2, and/or SEQ ID NO: 3.

24. The method of claim 22, wherein the double-stranded region comprises a nucleotide sequence that is substantially identical and substantially complementary to a mature miR-144 sequence.

25. The method of claim 22, wherein the double-stranded region comprises a nucleotide sequence which is at least 80%, 85%, 90%, 95% or 100% complementary to SEQ ID NO: 4.

26. The method of claim 5, wherein the agent is delivered to cells of the liver using any of:
a physical method, such as: parenteral administration, direct injection or electroporation;
a delivery vehicle such as: a glucan-containing particle, lipid containing vehicles, viral containing vehicles, polymer containing vehicles, peptide containing vehicles, and exosomes.

27. The agent for use, use or method of any of claims 1-26, wherein one or more symptoms of the liver disease and/or liver condition is improved in the subject following administration of the agent, for example hepatocyte death, immune cell infiltration and/or fibrosis.

28. The method of claim 3, wherein the agent is administered in combination with an additional therapy.

29. The method of claim 28 wherein the additional therapy is a lipid-lowering therapy, such as a HMG-CoA Reductase inhibitor.

30. The method of claim 12, wherein administration of the agent delays and/or prevents the progression from NASH to fibrosis, cirrhosis and/or hepatocellular carcinoma in the subject.

31. The method of claim 3, wherein the agent is formulated and/or adapted for delivery and/or uptake by cells of the liver.

32. The method of claim 3, wherein the agent is glucan encapsulated.

33. An agent that inhibits microRNA-144 (miR-144) for use in inhibiting progression of a liver disease and/or liver condition in which oxidative stress is a contributory factor in a subject.

34. (canceled)

35. A method for inhibiting progression of a liver disease and/or liver condition in which oxidative stress is a contributory factor in a subject, wherein the method comprises administering an agent that inhibits microRNA-144 (miR-144) to the subject.

36. A method for identifying a subject who is at risk of developing a liver disease and/or liver condition in which oxidative stress is a contributory factor, comprising:
a) obtaining and/or providing a test sample from a subject;
b) determining the expression and/or activity of miR-144 in the test sample,
wherein the expression and/or activity of miR-144 relative to a control indicates whether the subject is at risk of developing a liver disease and/or liver condition in which oxidative stress is a contributory factor.

37. A method for identifying a subject who has a liver disease and/or liver condition in which oxidative stress is a contributory factor, comprising:
a) obtaining and/or providing a test sample from a subject;
b) determining the expression and/or activity of miR-144 in the test sample,
wherein the expression and/or activity of miR-144 relative to a control sample indicates whether the subject has a liver disease and/or liver condition in which oxidative stress is a contributory factor.

38. A method for predicting the response of a subject having a liver disease and/or liver condition in which oxidative stress is a contributory factor, to treatment with an agent that inhibits microRNA-144 (miR-144), comprising:
a) obtaining and/or providing a test sample from a subject;
b) determining the expression and/or activity of miR-144 in the test sample,
wherein the expression and/or activity of miR-144 relative to a control sample indicates that the subject will respond to treatment with the agent.

39. The method of claim 36, wherein the method further comprises administering an effective amount of a therapy to the subject with a liver disease and/or liver condition in which oxidative stress is a contributory factor, for example wherein the method comprises administering an agent that inhibits miR-144.

40. (canceled)

41. (canceled)

42. The method of claim 36, or the use of any of claims 40-41, wherein the liver disease and/or liver condition in which oxidative stress is a contributory factor is as defined in any preceding claim.

43. (canceled)

44. (canceled)

45. The method of claim 36, wherein the liver disease and/or liver condition in which oxidative stress is a contributory factor in a subject is as defined in any preceding claim.

46. A pharmaceutical composition comprising an agent which inhibits miR-144, which is formulated and/or adapted for delivery to phagocytic cells of the liver.

47. The pharmaceutical composition of claim 46, wherein the agent is encapsulated for receptor-mediated uptake by phagocytic cells of the liver.

48. The pharmaceutical composition of claim 46, wherein the agent is as defined in any preceding claim.

49. The pharmaceutical composition of claim 46, wherein the composition is formulated for injection.

50. (canceled)

51. A kit of parts, comprising the pharmaceutical composition of claim 46.

52. (canceled)


SEQUENCE LISTING (40 sequences)

SEQ ID NO: 1 - RNA, 66 nt, Homo sapiens
ggcugggauaucaucauauacuguaaguuugugaugagacacuacaguauagaugauguacuaguc

SEQ ID NO: 2 - RNA, 23 nt, Homo sapiens
ggauaucaucauauacuguaagu

SEQ ID NO: 3 - RNA, 20 nt, Homo sapiens
uacaguauagaugauguacu

SEQ ID NO: 4 - skipped

SEQ ID NO: 5 - RNA, 66 nt, Mus musculus; identical to SEQ ID NO: 1

SEQ ID NO: 6 - RNA, 23 nt, Mus musculus
ggauaucaucauauacuguaagu

SEQ ID NO: 7 - RNA, 20 nt, Mus musculus
uacaguauagaugauguacu

SEQ ID NO: 8 - RNA, 21 nt, Artificial Sequence
  Synthetic antisense oligonucleotide
acuacaguauagaugaugucu

SEQ ID NO: 9 - RNA, 21 nt, Artificial Sequence
  Synthetic antisense oligonucleotide
acuacaguauagaugauaucu

SEQ ID NO: 10 - RNA, 21 nt, Artificial Sequence
  Synthetic antisense oligonucleotide
cuuacaguauaugaugauauc

SEQ ID NO: 11 - RNA, 20 nt, Artificial Sequence
  Synthetic antisense oligonucleotide
aguacaucaucuauacugua

SEQ ID NO: 12 - RNA, 27 nt, Artificial Sequence
  Synthetic antisense oligonucleotide
  misc_feature (1)..(10): n is a, c, g, or u
  misc_feature (18)..(27): n is a, c, g, or u
nnnnnnnnnnugaugucnnnnnnnnnn

SEQ ID NO: 13 - RNA, 27 nt, Artificial Sequence
  Synthetic antisense oligonucleotide
  misc_feature (1)..(10): n is a, c, g, or u
  misc_feature (18)..(27): n is a, c, g, or u
nnnnnnnnnnugauaucnnnnnnnnnn

SEQ ID NO: 14 - DNA, 19 bp, Artificial Sequence
  Synthetic primer
ccctacccagcctacatgg

SEQ ID NO: 15 - DNA, 22 bp, Artificial Sequence
  Synthetic primer
acatatcgagattggggtgtct

SEQ ID NO: 16 - DNA, 20 bp, Artificial Sequence
  Synthetic primer
ttctgtggcttccaggtctt

SEQ ID NO: 17 - DNA, 20 bp, Artificial Sequence
  Synthetic primer
aggctgcttggagcaaaata

SEQ ID NO: 18 - DNA, 22 bp, Artificial Sequence
  Synthetic primer
tgtcaccctcatctacaccaac

SEQ ID NO: 19 - DNA, 21 bp, Artificial Sequence
  Synthetic primer
cagggtctcaaaaggcttcag

SEQ ID NO: 20 - DNA, 23 bp, Artificial Sequence
  Synthetic primer
tctctgaggtggtttaccaaacg

SEQ ID NO: 21 - DNA, 20 bp, Artificial Sequence
  Synthetic primer
cctctcagacagcgcaccag

SEQ ID NO: 22 - DNA, 21 bp, Artificial Sequence
  Synthetic primer
tctacaatgagctgcgtgtgg

SEQ ID NO: 23 - DNA, 21 bp, Artificial Sequence
  Synthetic primer
gtacatggctggggtgttgaa

SEQ ID NO: 24 - DNA, 20 bp, Artificial Sequence
  Synthetic primer
cagcgacggaaagagtatga

SEQ ID NO: 25 - DNA, 18 bp, Artificial Sequence
  Synthetic primer
tgggcaacctgggagtag

SEQ ID NO: 26 - DNA, 21 bp, Artificial Sequence
  Synthetic primer
ggcagaagagcactgatcgta

SEQ ID NO: 27 - DNA, 22 bp, Artificial Sequence
  Synthetic primer
tgatgggattgaagttcatggc

SEQ ID NO: 28 - DNA, 20 bp, Artificial Sequence
  Synthetic primer
gtagtttgcccaaggtcaag

SEQ ID NO: 29 - DNA, 18 bp, Artificial Sequence
  Synthetic primer
agccacctgaggggtaag

SEQ ID NO: 30 - DNA, 18 bp, Artificial Sequence
  Synthetic primer
tcttcgcttgttgtgtcc

SEQ ID NO: 31 - DNA, 20 bp, Artificial Sequence
  Synthetic primer
cgaaggagaaaggcaatgac

SEQ ID NO: 32 - DNA, 20 bp, Artificial Sequence
  Synthetic primer
ttccagcaactccagcaacg

SEQ ID NO: 33 - DNA, 21 bp, Artificial Sequence
  Synthetic primer
cagattggctacccaactgtt

SEQ ID NO: 34 - DNA, 21 bp, Artificial Sequence
  Synthetic primer
gggaaggtgtaatccgtctcc

SEQ ID NO: 35 - DNA, 20 bp, Artificial Sequence
  Synthetic primer
cctgggctgtgcctgaccac

SEQ ID NO: 36 - DNA, 20 bp, Artificial Sequence
  Synthetic primer
agcactgtgaggggctgggg

SEQ ID NO: 37 - DNA, 25 bp, Artificial Sequence
  Synthetic primer
cgagatatacgcaggagaggtaaga

SEQ ID NO: 38 - DNA, 22 bp, Artificial Sequence
  Synthetic primer
gctcgacaatgttctccagctt

SEQ ID NO: 39 - DNA, 20 bp, Artificial Sequence
  Synthetic primer
gctgctgtgcccgtagtgag

SEQ ID NO: 40 - RNA, 22 nt, Caenorhabditis elegans
ucaccggguguaaaucagcuug